(19) United States
(12) Patent Application Publication
(10) Pub. No.: US 2018/0215829 A1
(43) Pub. Date: Aug. 2, 2018

(54) ANTI-IL-17RA IMMUNOGLOBULIN SINGLE HEAVY VARIABLE DOMAIN ANTIBODIES

(71) Applicant: CRESCENDO BIOLOGICS LIMITED, Cambridge (GB)

(72) Inventors: Bryan EDWARDS, Cambridge (GB); Ulla LASHMAR, Cambridge (GB); Brian MCGUINNESS, Cambridge (GB); Mike ROMANOS, Cambridge (GB); Thomas SANDAL, Cambridge (GB); Joyce Young, Cambridge (GB)

(21) Appl. No.: 15/541,876

(22) PCT Filed: Jan. 12, 2016
(86) PCT No.: PCT/GB2016/050067

§ 371 Date: Jul. 6, 2017

(30) Foreign Application Priority Data
Jan. 12, 2015 (GB) 1500461.7

(51) Int. Cl. C07K 16/28; G01N 33/68; A01K 67/027

(52) U.S. Cl. CPC C07K 16/2866; G01N 33/6869; A01K 67/0278; C07K 2317/92; C07K 2317/569; A01K 2217/15; C07K 2317/76; C07K 2317/565

(57) ABSTRACT

Binding molecules that bind specifically to IL-17RA. The binding molecules are useful in the treatment of disease, for example psoriasis.

### FIELD OF THE INVENTION

The invention relates to IL-17RA binding molecules, and the use of such binding molecule in the treatment of disease.

### INTRODUCTION

Psoriasis is a chronic relapsing and remitting inflammatory skin disease affecting 2-3% of the world's population (˜125 m sufferers) that causes significant morbidity and decreased quality of life, largely due to clinical flare-ups and disfiguring lesions in visible areas of the skin, systemic manifestations and drug-related side effects. The common form of the disease, termed ‘plaque psoriasis vulgaris’, is observed in more than 80% of patients and is characterized by erythematous scaly plaques (typically on elbows, knees, scalp and buttocks) which can vary in size from minimal to the involvement of the entire skin surface.

Depending on the degree of body surface area (BSA) involvement, psoriasis can be categorised into mild (<3% BSA involvement), moderate (3-10% BSA) and severe (>10% BSA) disease. Topical agents such as corticosteroids, vitamin D derivatives, coal tar and topical retinoids are the cornerstone of the initial management of psoriasis and are an important part of the treatment ladder applied to patients across the spectrum of disease severity. Patients diagnosed with mild-to-moderate disease are typically prescribed topical agents as monotherapy. Patients with severe disease are typically prescribed topical agents as an adjunct to phototherapy or systemic (small molecule) therapies such as methotrexate, cyclosporine or oral retinoids etc. The treatment regime for moderate-to-severe psoriasis also includes antibody-based therapies.

In recent years the importance of the Th17 pathway has become well validated in psoriasis and several monoclonal antibodies (mAbs) targeting components of this pathway (including IL17RA) have shown the significant importance of modulating this pathway and influencing psoriasis. IL-17RA is one of a family of related receptors (named IL-17RA, to IL-17RE) which multimerise to form signalling complexes. Each receptor complex exhibits differential binding to one of a range of related ligands (IL-17A, IL-17B, IL-17C, IL-17D, IL-17E or IL-17F). In their active form, all of the ligands are covalent homodimers (except IL-17A and IL-17F which are also known to heterodimerise). It is thought that IL-17A, IL-17F and IL-17A/IL-17F all signal through the same receptor subunits, IL-17RA and IL-17RC, which together form a heteromeric complex. Nonetheless, IL-17A and IL-17F have distinct biological effects. Studies comparing Il17a^{-/-} mice with Il17f^{-/-} mice indicate that IL-17A plays a central role in driving autoimmunity (in particular the pathology associated with psoriasis) and that it does so through primarily through signaling via IL-17RA. The role of IL-17A/F heterodimers is still to be fully elucidated. While psoriasis may have a systemic component in some patients, the disease is primarily one of the skin. IL-17 secreted by Th17 cells acts on epidermal keratinocytes, via IL-17R complexes present on these cells, to initiate a feedback loop of keratinocyte hyper-proliferation and on-going inflammation, thereby generating the psoriatic plaque. It is believed that the primary element of pathological activity is locally in the skin, and therefore inhibition of the IL-17/IL-17R interaction is the best validated target for topical therapy. This is in contrast to other validated Th17 targets, such as IL-23, where a significant phase of activity is in regional lymph nodes.

Current treatments for psoriasis include topical agents such as corticosteroids, vitamin D derivatives, coal tar and topical retinoids, these are the cornerstones of the initial management of psoriasis (Nast et al., Arch Dermatol Res (2007) 299:111-138) and, depending on disease severity, are typically prescribed as monotherapy.

Patients with severe disease are typically prescribed topical agents as an adjunct to phototherapy or systemic (small molecule) therapies such as methotrexate, cyclosporine or oral retinoids (Nast et al., Arch Dermatol Res (2007) 299:111-138). Phototherapy can be effective but is inconvenient and associated with a significant risk of skin cancer. Small molecule systemic therapies are associated with increased cardiovascular risk; renal dysfunction, leucopenia and thrombocytopenia. For example, methotrexate may cause a neutropenia and liver damage and is contraindicated for males and females of reproductive age without due precaution. Cyclosporine is a potent immunosuppressant, which has potential adverse effects on the kidneys and blood pressure. Acitretin is an oral retinoid that has a range of side effects, and is also contraindicated for females of reproductive age without due precaution (Nast et al. Arch Dermatol Res (2007) 299:111-138).

The treatment regimen for moderate-to-severe psoriasis also includes antibody-based therapies. Approved treatments include adalimumab (Humira®), a humanized monoclonal antibody with activity against TNF-alpha(α), the TNF-α inhibitor etanercept (Enbrel®), the TNF-α inhibitor infliximab (Remicade®) and most recently ustekinumab (Stelara®), a human mAb that targets the common p40 subunit of IL12 and IL23, thereby blocking the signalling of both cytokines.

Systemic biologics have transformed treatment of moderate-to-severe psoriasis but, as with any immunosuppressive regimen, chronic use can have significant side-effects such as increased risk of infections or malignancies. Thus, there is a need for new highly effective and safe therapy options for both topical and systemic use.

Several other monoclonal antibodies agents in development have been shown to markedly reduce disease severity in patients with moderate-to-severe plaque psoriasis. These agents include ixekizumab (Eli Lilly) and secukinumab (Novartis), both of which target IL-17A, and brodalumab (Amgen) that binds to and inhibits signalling of IL-17RA and therefore would be expected to block IL-17 family members that utilize this receptor, including IL-17A, IL-17F, IL-17A/F and possibly IL-17E.

The clinical results for the IL17-R inhibitor brodalumab indicate the importance of IL17-RA in psoriasis pathophysiology. In independent clinical studies up to and including significant Phase II trials, it has been reported markedly to reduce disease severity in patients with moderate-to-severe plaque psoriasis, and is said to demonstrate a favorable safety and tolerability profile, robust clinical activity, significant improvements in PASI and other scores for psoriasis severity, and a substantial positive impact on patient quality of life (Papp K A et al. N Engl J Med. 2012; 366(13):1181-1189; Papp K A et al. J Invest Dermatol. 2012; 132(10):2466-2469; Gordon K B et al. Br J Dermatol. 2014; 170(3):705-715)

Similarly, inhibition of IL-17A (the major cytokine signaling through IL-17RA) by several antibody antagonists in clinical development have been shown to be highly effective for the treatment of patients with moderate-severe psoriasis. In particular, secukinumab (currently in substantial phase III clinical studies) has been shown to down-regulate cytokines, chemokines and proteins associated with inflammatory responses in lesional skin.

The therapeutic products currently on the market for the treatment of psoriasis offer varying degrees of symptomatic relief and reduced relapse rates but none are currently considered curative and therefore require chronic administration. While many pre-existing topical agents can be effective for short periods of time, due to treatment-limiting toxicity most are restricted to short term use. This means that patients need routine monitoring for side effects and regular cycling onto new treatment protocols. Phototherapy can be effective but is inconvenient and associated with a significant risk of skin cancer and many conventional (small molecule) systemic therapies are associated with increased cardiovascular risk; renal dysfunction, leucopenia and thrombocytopenia. Systemic biologics have transformed treatment of moderate-to-severe psoriasis but, as with any immunosuppressive regime, chronic use can have significant side-effects such as increased risk of infections or malignancies.

None of the current therapeutic interventions are curative, and therefore all require chronic use. Therapeutic regimens have to take account of this by adopting strategies to reduce toxicity, including rotational or sequential therapies, drug holidays, and combination therapy. Importantly, for some drugs there is an absolute lifetime limit on the exposure that any one patient can safely receive.

Thus, there is a need for new highly effective and safe therapy options for both topical and systemic use. In particular, there is therefore a clear unmet need for new topical drugs with the efficacy of a biological in patients with severe disease, where a long-term maintenance therapy could keep symptoms under control following systemic mAb use and therefore improve the safety profile for chronic use. Similarly, those patients who are not treated systemically because their disease is considered sufficiently severe, would greatly benefit from the topical, in particular dermal, application of a drug with biological efficacy.

Antibodies have proven themselves to be extremely effective therapeutic agents for treating a large number of different disease indications. In particular, there has been a clear trend towards development of fully human antibodies for therapeutic use over the various alternatives. Due to their size and other physical properties, however, it is currently the case that monoclonal antibodies have to be administered either intravenously (iv) or subcutaneously (sc) and therefore have a high systemic exposure. Thus, although the antibodies can be highly effective, their route of delivery can often be suboptimal, resulting either in antibody binding to target antigen at non-disease locations (potentially compromising the healthy function of normal, non-disease tissue) or resulting in suboptimal PK/PD characteristics. Either outcome may result in a loss of efficacy and/or a compromised safety profile by virtue of the suboptimal route of administration.

Due to their small size and other favourable biophysical characteristics, antibody fragments are potentially attractive candidates for alternative routes of administration. In particular, V_{H} fragments are the smallest, most robust portion of an immunoglobulin molecule that retain target specificity and potency. It would therefore be advantageous to deliver V_{H} domain therapeutics topically on the skin, so that they penetrate to therapeutically beneficial locations within the skin to treat disease locally. Any V_{H} that might enter the bloodstream will be cleared rapidly and therefore have little or no systemic exposure, thereby minimising potential mechanism-related systemic toxicity.

The invention is thus aimed at providing a safe and effective therapy of conditions associated with the Th17 pathway, in particular for topical treatment of psoriasis.

### SUMMARY OF THE INVENTION

The invention relates to isolated IL-17RA binding molecules, related uses and methods, including their use in medical treatment.

In a first aspect, the invention relates to a binding molecule capable of binding human IL-17RA comprising a human heavy chain variable immunoglobulin domain (V_{H}) comprising a CDR3 sequence comprising SEQ ID NO. 3 or a sequence with at least 70%, at least 80%, at least 90%, or at least 95% homology to SEQ ID NO. 3.

In a second aspect, the invention relates to a binding molecule, comprising at least one immunoglobulin single domain antibody directed against IL-17RA wherein said domain is a human V_{H} domain comprising at least one antigen binding site comprising a CDR3 sequence having SEQ ID NO. 3 or a sequence with at least 70%, at least 80%, at least 90%, or at least 95% homology to SEQ ID NO. 3. In a third aspect, the invention relates to a binding molecule capable of binding human IL-17RA comprising a human V_{H} domain comprising a CDR3 sequence comprising SEQ ID NO. 1267 or a sequence with at least 70%, at least 80%, at least 90%, or at least 95% homology to SEQ ID NO. 1267.

In a fourth aspect, the invention relates to a binding molecule comprising at least one immunoglobulin single domain antibody directed against human IL-17RA wherein said domain is a human V_{H} domain comprising an antigen binding site comprising a CDR3 sequence having SEQ ID NO. 1267 or a sequence with at least 70%, at least 80%, at least 90%, or at least 95% homology to SEQ ID NO. 1267.

In a fifth aspect, the invention relates to a binding molecule capable of binding human IL-17RA comprising a human V_{H} domain comprising a CDR3 sequence comprising SEQ ID NO. 1767 or a sequence with at least 70%, at least 80%, at least 90%, or at least 95% homology to SEQ ID NO. 1767.

In a another aspect, the invention relates to a binding molecule comprising at least one immunoglobulin single domain antibody directed against human IL-17RA wherein said domain is a human V_{H} domain comprising at least one antigen binding site comprising a CDR3 sequence having SEQ ID NO. 1767 or a sequence with at least 70%, at least 80%, at least 90%, or at least 95% homology to SEQ ID NO. 1767.

In another aspect, the invention relates to a binding molecule capable of binding human IL-17RA comprising a human V_{H} domain comprising a CDR3 sequence comprising SEQ ID NO. 2131 or a sequence with at least 70%, at least 80%, at least 90%, or at least 95% homology to SEQ ID NO. 2131.

In a fourth aspect, the invention relates to a binding molecule comprising at least one immunoglobulin single domain antibody directed against human IL-17RA wherein said domain is a human V_{H} domain comprising an antigen binding site comprising a CDR3 sequence having SEQ ID NO. 2131 or a sequence with at least 60%, at least 70%, at least 90%, or at least 95% homology to SEQ ID NO. 2131.

In another aspect, the invention relates to a binding molecule capable of binding human IL-17RA comprising a human V_{H} domain comprising a CDR3 sequence comprising SEQ ID NO. 2559 or a sequence with at least 70%, at least 80%, at least 90%, or at least 95% homology to SEQ ID NO. 2559.

In another aspect, the invention relates to a binding molecule comprising at least one immunoglobulin single domain antibody directed against human IL-17RA wherein said domain is a human V_{H} domain comprising at least one antigen binding site comprising a CDR3 sequence having SEQ ID NO. 2559 or a sequence with at least 70%, at least 80%, at least 90%, or at least 95% homology to SEQ ID NO. 2559.

In another aspect, the invention relates to a binding molecule capable of binding human IL-17RA comprising a human V_{H} comprising a CDR3 sequence comprising SEQ ID NO. 2575 or a sequence with at least 70%, at least 80%, at least 90%, or at least 95% homology to SEQ ID NO. 2575.

In another aspect, the invention relates to a binding molecule comprising at least one immunoglobulin single domain antibody directed against human IL-17RA wherein said domain is a human V_{H} domain comprising at least one antigen binding site comprising a CDR3 sequence having SEQ ID NO. 2575 or a sequence with at least 70%, at least 80%, at least 90%, or at least 95% homology to SEQ ID NO. 2575.

In another aspect, the invention relates to a binding molecule capable of binding human IL-17RA comprising a human V_{H} domain comprising a CDR3 sequence comprising SEQ ID NO. 2579 or a sequence with at least 70%, at least 80%, at least 90%, or at least 95% homology thereto.

In another, the invention relates to a binding molecule comprising at least one immunoglobulin single domain antibody directed against human IL-17RA wherein said domain is a human V_{H} domain comprising at least one antigen binding site comprising a CDR3 sequence having SEQ ID NO. 2579 or a sequence with at least 70%, at least 80%, at least 90%, or at least 95% homology to SEQ ID NO. 2579.

In another aspect, the invention relates to a binding molecule comprising an immunoglobulin single domain antibody directed against human IL-17RA wherein the binding molecule has an IC50 for inhibition of IL-6 production of about 0.2 to about 500 nM when tested as described in the examples, i.e. by measuring the ability of IL-17R-binding molecule to inhibit IL-17R induced IL-6 release from the cell line HT1080.

In another aspect, the invention relates to a binding molecule comprising an immunoglobulin single domain antibody directed against human IL-17RA wherein said binding molecule has a KD (M) value in the range of from 6×10-11 to 3×10-7, preferably in the range of from 1×10⁻⁹ to 6×10⁻¹¹, preferably when assessed by BIAcore®.

In another aspect, the invention relates to a pharmaceutical composition comprising a binding molecule as described above and a pharmaceutical carrier.

In another aspect, the invention relates to a method for treating a disease selected from autoimmune diseases, inflammatory conditions, allergies and allergic conditions, hypersensitivity reactions, severe infections, and organ or tissue transplant rejection comprising administering to a patient in need thereof a binding molecule or pharmaceutical composition of the invention.

In another aspect, the invention relates to a binding molecule or a pharmaceutical composition of the invention for use in the treatment of a disease selected from an autoimmune disease, inflammatory conditions, allergies and allergic conditions, hypersensitivity reactions, severe infections, and organ or tissue transplant rejection.

In another aspect, the invention relates to the use of a binding molecule or a pharmaceutical composition of the invention in the manufacture of a medicament for the treatment of a disease selected from an autoimmune disease, inflammatory conditions, allergies and allergic conditions, hypersensitivity reactions, severe infections, and organ or tissue transplant rejection.

In another aspect, the invention relates to an in vivo or in vitro method for reducing human IL-17RA activity comprising contacting human IL-17RA with a binding molecule as described above.

In another aspect, the invention relates to a method for determining the presence of human IL-17RA in a test sample by an immunoassay comprising contacting said sample with a binding molecule as described above and at least one detectable label.

In another aspect, the invention relates to an isolated nucleic acid molecule comprising a nucleotide sequence encoding a binding molecule of the invention.

In another aspect, the invention relates to an isolated nucleic acid construct comprising a nucleic acid as described above.

In another aspect, the invention relates to an isolated host cell comprising a nucleic acid or a construct as described above.

In another aspect, the invention relates to a method for producing a binding molecule as described above comprising expressing a nucleic acid encoding said binding molecule in a host cell and isolating the binding molecule from the host cell culture.

In another aspect, the invention relates to kit comprising a binding molecule or a pharmaceutical composition of the invention as described above

### DRAWINGS

FIG. 1. Family 1 sequences. This figure shows the full length V_{H} sequence for clones in family 1. Framework (FR) and complementarity-determining regions (CDR) are labelled and shown in table form for ease of reference. CDR1, CDR2 and CDR3 are highlighted in bold.

FIG. 2. Family 2 sequences. This figure shows the full length V_{H} sequence for clones in family 2. Framework (FR) and complementarity-determining regions (CDR) are labelled and shown in table form for ease of reference. CDR1, CDR2 and CDR3 are highlighted in bold.

FIG. 3. Family 3 sequences. This figure shows the full length V_{H} sequence for clones in family 3 and shown in table form for ease of reference. Framework (FR) and complementarity-determining regions (CDR) are labelled, CDR1, CDR2 and CDR3 are highlighted in bold.

FIG. 4. Family 4 sequences. This figure shows the full length V_{H} sequence for clones in family 4 and shown in table form for ease of reference. Framework (FR) and complementarity-determining regions (CDR) are labelled. CDR1, CDR2 and CDR3 are highlighted in bold.

FIG. 5. Family 5 sequences. This figure shows the full length V_{H} sequence for clones in family 5 and shown in table form for ease of reference. Framework (FR) and complementarity-determining regions (CDR) are labelled. CDR1, CDR2 and CDR3 are highlighted in bold.

FIG. 6. Family 6 sequences. This figure shows the full length V_{H} sequence for clones in family 6. Framework (FR) and complementarity-determining regions (CDR) are labelled. CDR1, CDR2 and CDR3 are highlighted in bold.

FIG. 7. Family 7 sequences. This figure shows the full length V_{H} sequence for clones in family 7. Framework (FR) and complementarity-determining regions (CDR) are labelled. CDR1, CDR2 and CDR3 are highlighted in bold.

FIG. 8 shows serum ELISA data, confirming immunogen-induced heavy chain antibody response.

FIG. 9 shows in vitro selection Mouse ELISA data: (A) V_{H} isolated from peripreps binding to IL-17RA, (B) V_{H} isolated from phage preparations binding to IL-17RA and (C) V_{H} isolated from phage preparations binding to human IgG1.

FIG. 10 shows the results of biochemical assays: IL-17RA ligand inhibition assays.

A: the x-axis shows the concentration of V_{H} (M), the y-axis shows the OD₄₅₀ nm, for V_{H} 49G11 (●) the IC₅₀ (nM) was 900 and for V_{H} 2.1 (▪) the IC₅₀ (nM) was 4.

B: the x-axis shows V_{H} concentration M (log)₁₀, the y-axis shows the OD₄₅₀ nm, for V_{H} 4.55 (●) the IC₅₀ (nM) was 6928; for V_{H} 3.1 (▪) the IC₅₀ (nM) was 11; for V_{H} 3.20 (▴), the IC₅₀ (nM) was 22; for V_{H} 49G11 (▾), the IC₅₀ (nM) was 885.

FIG. 11 shows the results of cell-based assays for IL-17RA V_{H} clones.

A: the x-axis shows V_{H} concentration M (log)₁₀, the y-axis shows the OD₄₅₀ nm; V_{H} SEV49G11 (●) had a weak IC₅₀ (nM), V_{H} 2.1 (▪) had an IC₅₀ (nM) of 363, V_{H} 62A4 (▴) (QVQLVESGGGLVQPGRSLTLSCTASGFTFHDYAMHWVRQPPGGGLEVWAGVSWN GNNVGYADSVKGRFTISRDNAKKSLYLQMNSLRSEDTALYYCAKGGMGSGSHPDSF STWGQGTMVTVSS, SEQ ID No. 2604) had a weak IC₅₀ (nM), V_{H} SEV136H4L (∇) had an IC₅₀ (nM) of 5; no IC₅₀ (nM) was recorded for V_{H} 846A5 (◯).

B: the x-axis shows V_{H} concentration M (log)₁₀, the y-axis shows the OD₄₅₀ nm; MAB177 (●) had an IC₅₀ (nM) of 65, V_{H} 2.2 (▪) had an IC₅₀ (nM) of 165, V_{H} 1.1 (▴) had an IC₅₀ (nM) of 39, V_{H} 1.2 (▾) had an IC₅₀ (nM) of 141, no IC₅₀ (nM) was recorded for LH86A5 (◯).

FIG. 12 shows the BIAcore™ traces for IL-17R V_{H} (A) Clone 2.1, (B) Clone 2.2, (C) clone 1.1 and (D) clone 1.2

FIG. 13 shows clone 2.1 V_{H} family optimisation, the full sequence of clone 2.1 is shown as the top line in bold (SEQ ID NO: 1268). Figure also discloses SEQ ID NOS 1640, 1632, 1664, 1644, 1636, 1668, 1764, 1720, 1724, 1376, 1760, 1692, 1708, 1688, 1728, 1684, 1676, 1672, 1744, 1748, 1648, 1696, 1716, 1712, 1704, 1660, 1752, 1652, 1656, 1736, 1680, 1700, 1756, 1740, and 1732, respectively, in order of appearance.

FIG. 14 shows specificity ELISAs for clones 1.2, 1.1, 2.1, 62A4 and 86A5.

FIG. 15 shows epitope competition for IL-17RA V_{H} clones V_{H} 1.1 and 2.2, which bind to different epitopes on IL-17RA.

FIG. 16 shows HPLC SEC for IL-17 RA for clones (A) 2.1, (B) 1.2 and (C) 1.1.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Sambrook et al. Molecular Cloning: A Laboratory Manual (2d ed., Cold Spring Harbor Laboratory Press, Cold Spring Harbor, N.Y. (1989)). Enzymatic reactions and purification techniques are performed according to the manufacturer's specifications, as commonly accomplished in the art or as described herein. The nomenclatures used in connection with, and the laboratory procedures and techniques of, analytical chemistry, synthetic organic chemistry, medicinal and pharmaceutical chemistry described herein are those well-known and commonly used in the art. Standard techniques are used for chemical syntheses, chemical analyses, pharmaceutical preparation, formulation, and delivery, and treatment of patients.

The IL-17 family of cytokines includes six members, IL-17/IL-17A, IL-17B, IL-17C, IL-17D, IL-17E/IL-25, and IL-17F, which are produced by multiple cell types. Members of this family have a highly conserved C-terminus containing a cysteine-knot fold structure. Most IL-17 proteins are secreted as disulfide-linked dimers, with the exception of IL-17B, which is secreted as a non-covalent homodimer.

Signaling by IL-17 family cytokines is mediated by members of the IL-17 receptor family (IL-17R), IL-17 R/IL-17 RA, IL-17 B R/IL-17 RB, IL-17 RC, IL-17 RD, and IL-17 RE. Activation of these receptors triggers intracellular pathways that induce the production of pro-inflammatory cytokines and anti-microbial peptides. IL-17A, IL-17F, and IL-17A/F are produced primarily by activated T cells and signal through an oligomerized receptor complex consisting of IL-17 RA and IL-17 RC. Ligand binding to this complex leads to recruitment of the intracellular adaptor proteins, Act1 and TRAF-6, and downstream activation of the transcription factors, NF kappa B, AP-1, and C/EBP. IL-17E activates similar signaling pathways through a receptor complex formed by IL-17 RA and IL-17 B R/IL-17RB. Signaling by IL-17E induces Th2-type immune responses and may be involved in promoting the pathogenesis of asthma. Less is known about the signaling pathways activated by other IL-17 family cytokines. Recent studies suggest that IL-17C is produced primarily by epithelial cells and binds to a receptor complex consisting of IL-17 RA and IL-17 RE. Autocrine signaling by IL-17C in epithelial cells stimulates the production of anti-microbial peptides and pro-inflammatory cytokines, but like IL-17A, overexpression of IL-17C may contribute to the development of autoimmune diseases. Similar to IL-17E, IL-17B binds to IL-17 B R/IL-17 RB, but the major target cells and effects of IL-17B signaling have not been reported. In addition, the receptor for IL-17D and the ligand for IL-17 RD are currently unknown.

The invention provides isolated IL-17RA binding molecules that bind human IL-17RA, pharmaceutical compositions comprising such binding molecules, as well as isolated nucleic acids encoding such binding molecules, recombinant expression vectors and isolated host cells for making such binding proteins. Also provided by the invention are methods of using the binding molecules disclosed herein to detect human IL-17RA, to inhibit human IL-17RA either in vitro or in vivo, and methods of treating disease. One aspect of the invention provides isolated human anti-human IL-17RA binding molecules, specifically those comprising, or consisting of, single domain antibodies that bind to human IL-17RA with high affinity, a slow off rate and high neutralizing capacity. In one embodiment, the binding molecule is a heavy chain only antibody.

In preferred embodiments, the binding molecules of the invention bind specifically to human IL-17RA and do not cross react with, or do not show substantial binding to, other members of the human IL-17R receptor family. This limited cross-reactivity with IL-17R homologues exhibited by the binding members of the invention offers advantages for their therapeutic and/or diagnostic use as side effects by undesirable cross reactivity are reduced. This also offers advantages in dosing for therapeutic applications.

Binding molecules of the invention are isolated from their natural environment.

An IL-17RA binding molecule of the invention is directed against, that is capable of binding to human IL-17RA (Protein accession NO. Q96F46 Uniprot, SEQ ID NO. 2601) showing the full-length precursor IL-17RA including the signal peptide) and/or cynomolgus monkey IL-17R.

SEQ ID NO. 2603
MGAARSPPSAVPGPLLGLLLLLLGVLAPGGASLRLLDHRALVCSQPG
LNCTVKNSTCLDDSWIHPRNLITSSPKDLQIQLHFAHTQQGDLFPVA
HIEWTLQTDASILYLEGAELSVLQLNTNERLCVRFEFLSKLRHHHRR
WRFTFSHFVVDPDQEYEVTVHHLPKPIPDGDPNHQSKNFLVPDCEHA
RMKVTTPCMSSGSLWDPNITVETLEAHQLRVSFTLWNESTHYQILLT
SFPHMENHSCFEHMHHIPAPRPEEFHQRSNVTLTLRNLKGCCRHQVQ
IQPFFSSCLNDCLRHSATVSCPEMPDTPEPIPDYMPLWVYWFITGIS
ILLVGSVILLIVCMTWRLAGPGSEKYSDDTKYTDGLPAADLIPPPLK
PRKVWIIYSADHPLYVDVVLKFAQFLLTACGTEVALDLLEEQAISEA
GVMTWVGRQKQEMVESNSKIIVLCSRGTRAKWQALLGRGAPVRLRCD
HGKPVGDLFTAAMNMILPDFKRPACFGTYVVCYFSEVSCDGDVPDLF
GAAPRYPLMDRFEEVYFRIQDLEMFQPGRMHRVGELSGDNYLRSPGG
RQLRAALDRFRDWQVRCPDWFECENLYSADDQDAPSLDEEVFEEPLL
PPGTGIVKRAPLVREPGSQACLAIDPLVGEEGGAAVAKLEPHLQPRG
QPAPQPLHTLVLAAEEGALVAAVEPGPLADGAAVRLALAGEGEACPL
LGSPGAGRNSVLFLPVDPEDSPLGSSTPMASPDLLPEDVREHLEGLM
LSLFEQSLSCQAQGGCSRPAMVLTDPHTPYEEEQRQSVQSDQGYISR
SSPQPPEGLTEMEEEEEEEQDPGKPALPLSPEDLESLRSLQRQLLFR
QLQKNSGWDTMGSESEGPSA

The terms “IL-17R binding molecule”, “IL-17R binding protein”, “anti-IL-17R single domain antibody” or “anti-IL-17R antibody” all refer to a molecule capable of binding to/directed to the IL-17RA antigen. Thus, unless otherwise stated, the term human IL-17R refers to human IL-17RA. The binding reaction may be shown by standard methods (qualitative assays) including, for example, a binding assay, competition assay or a bioassay for determining the inhibition of IL-17R binding to its receptor or any kind of binding assays, with reference to a negative control test in which an antibody of unrelated specificity. The term “IL-17R binding molecule” includes an IL-17R binding protein or peptide.

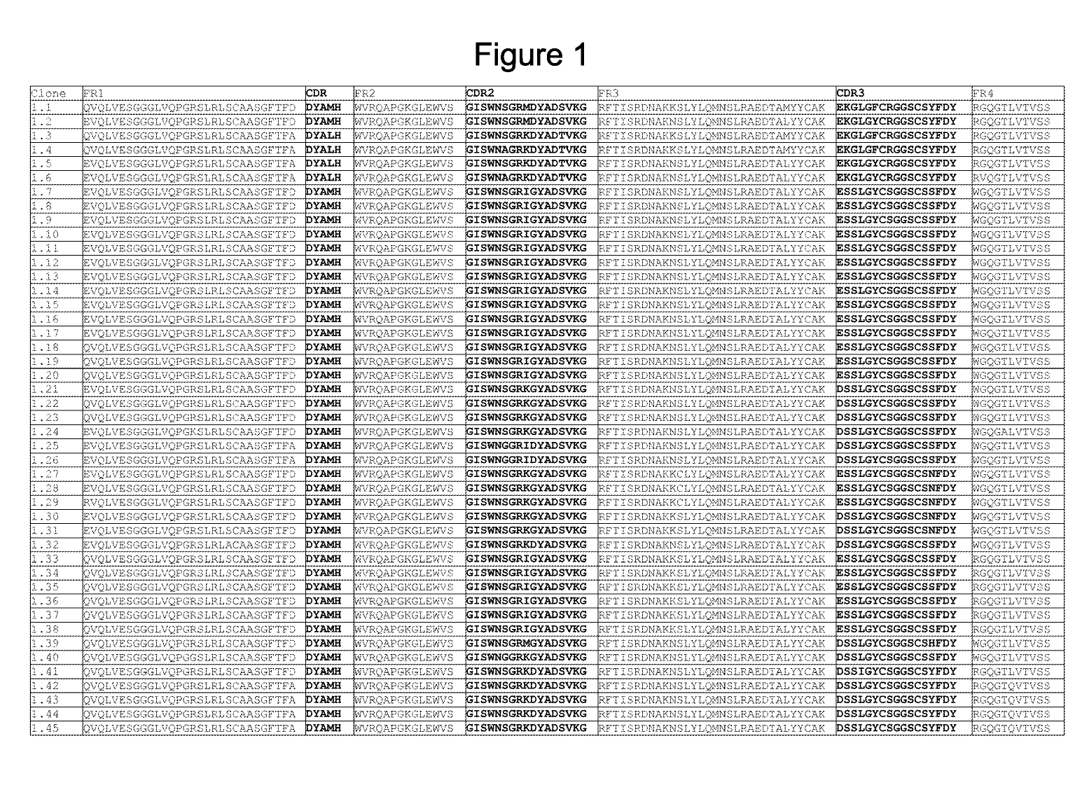

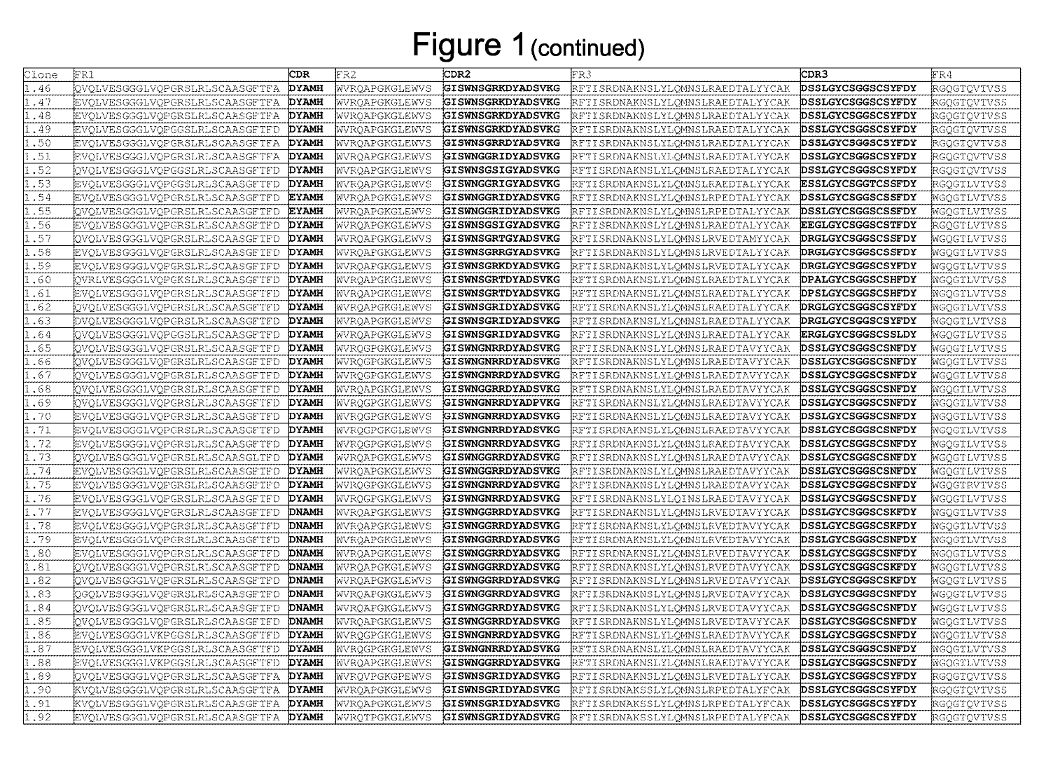

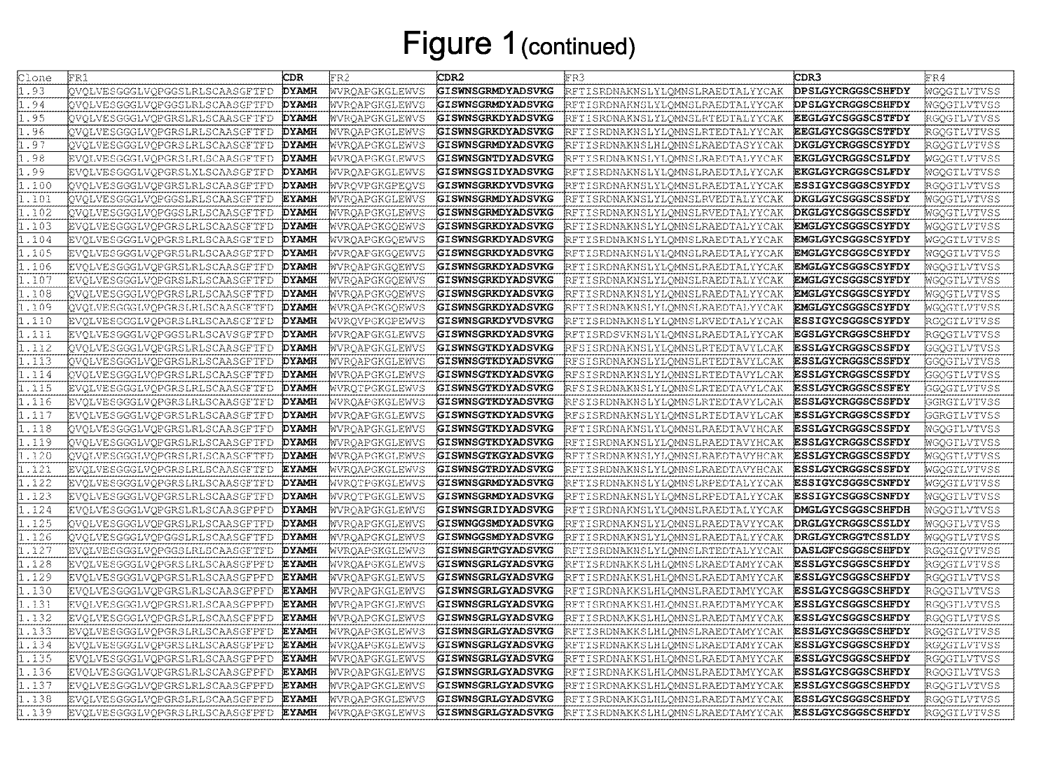

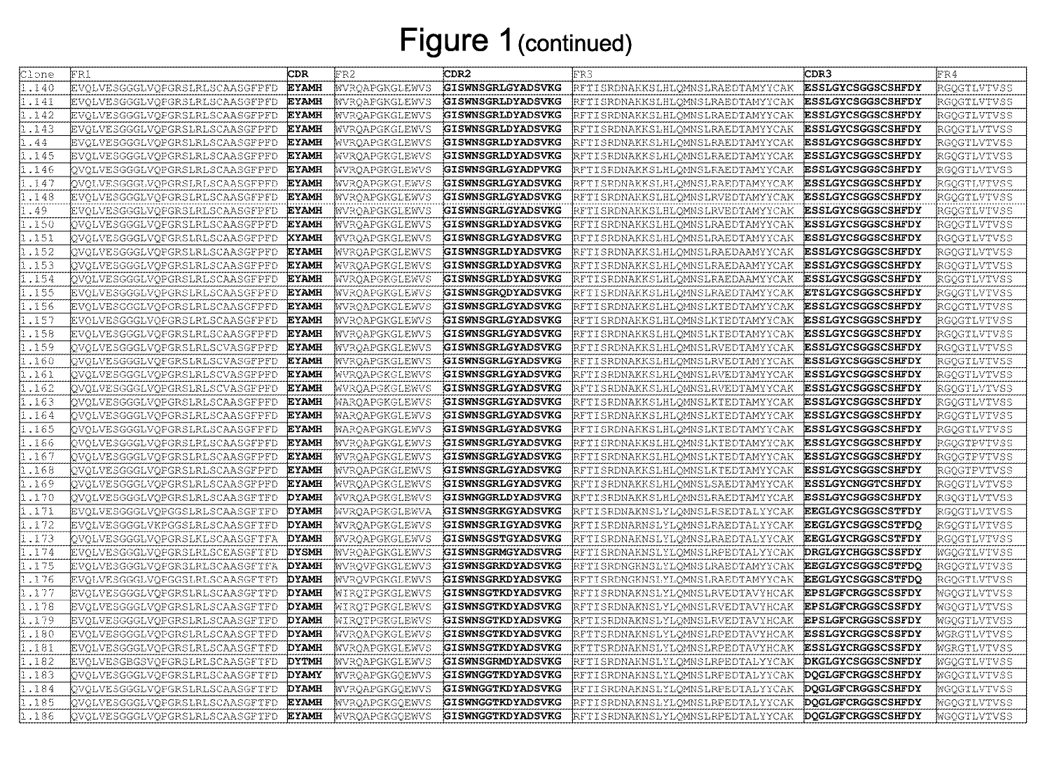

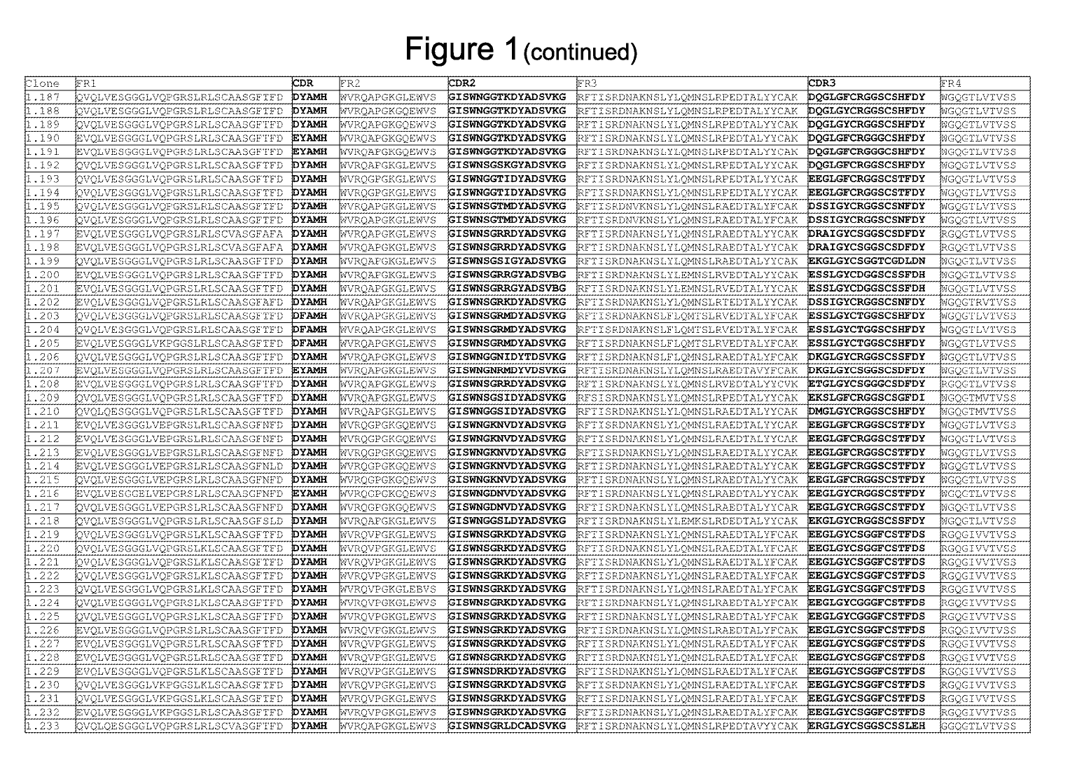

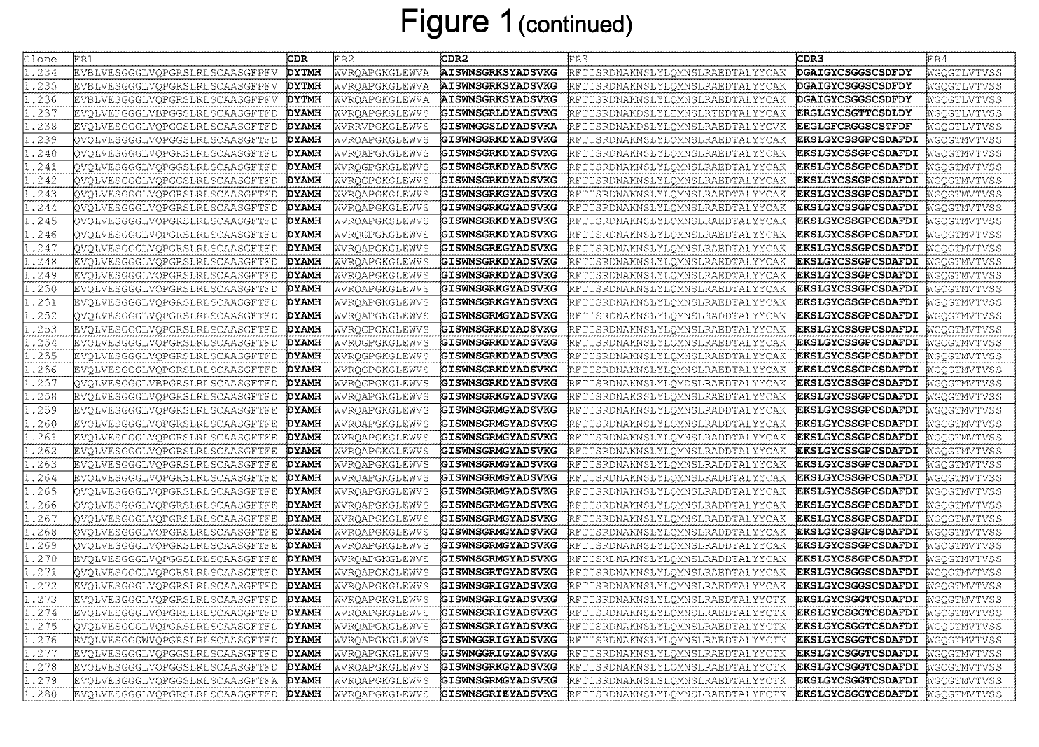

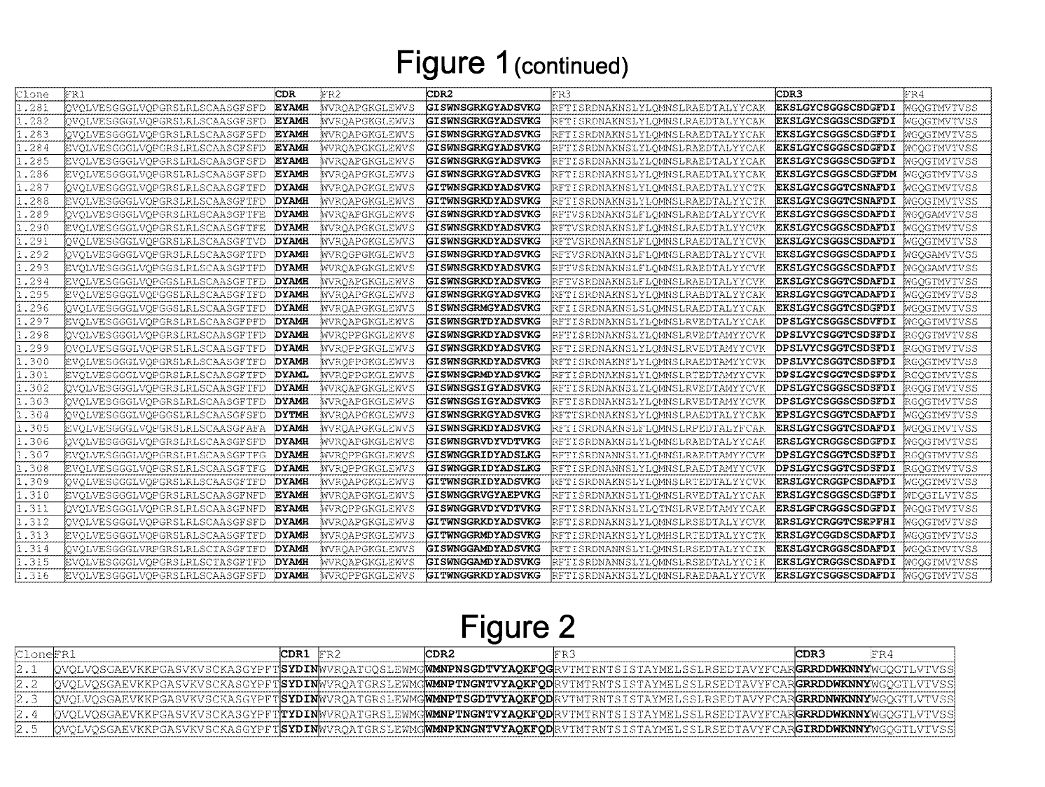

The invention relates to isolated binding molecules capable of binding to human IL-17RA comprising a heavy chain variable immunoglobulin domain (V_{H}) comprising a CDR3 sequence as shown in any of FIGS. 1 to 7 with reference to tables 1 to 7 or a sequence with at least 70%, at least 80%, at least 90%, or at least 95% homology thereto. In one embodiment, the binding molecule comprises a set of CDR1, 2 and 3 sequences selected from the sets of CDR1, 2 and 3 sequences as shown in any of FIGS. 1 to 7 with reference to tables 1 to 7. In one embodiment, the binding molecule comprises a V_{H} with a set of CDR1, 2 and 3 sequences selected from the sets of CDR1, 2 and 3 sequences as shown in any of FIGS. 1 to 7 with reference to tables 1 to 7. In one embodiment, the binding molecule comprises a heavy chain only antibody.

In another aspect, the invention relates to an isolated binding molecule comprising at least one immunoglobulin single domain antibody directed against/capable of binding to IL-17RA wherein said domain is a V_{H} domain and wherein said IL-17RA binding molecule comprises at least one antigen binding site.

In one embodiment, the binding molecule may comprise at least one single domain antibody directed against IL-17RA wherein said domain is a V_{H} domain comprising a CDR3 as shown in any of FIGS. 1 to 7 with reference to tables 1 to 7 or a sequence with at least 60%, at least 70%, at least 80%, at least 90%, or at least 95% homology to said CDR3 or said V_{H}.

In one embodiment, said at least one single variable heavy chain domain antibody comprises a set of CDR1, 2 and 3 sequences or a V_{H} a set of CDR1, 2 and 3 sequences wherein the CDR sequences are selected from the sets of CDR1, 2 and 3 sequences as shown in any of FIGS. 1 to 7 with reference to tables 1 to 7. In another embodiment, the binding molecules comprises or consists of a V_{H} domain as shown for a clone selected from clones 1.1 to 1.316, 2.1 to 2.125, 3.1 to 3.91, 4.1 to 4.107, 5.1 to 5.4, 6.1 or 7.1.

In one embodiment of the aspects above, said homology is at least 61%, 62%, 63%, 64%, 65%, 66%, 67%, 68%, 69%, 70%, 71%, 72%, 73%, 74%, 75%, 76%, 77%, 78%, 79%, 80%, 82%, 82%, 83%, 84%, 85%, 86%, 87%, 88%, 89%, 90%, 91%, 92%, 93%, 94%, 95%, 96%, 97%, 98% or 99%.

“Homology” generally refers to the percentage of amino acid residues in the candidate sequence that are identical with the residues of the polypeptide with which it is compared (sequence identity), after aligning the sequences and in some embodiments after introducing gaps, if necessary, to achieve the maximum percent homology, and not considering any conservative substitutions as part of the sequence identity. Neither N- or C-terminal extensions, tags or insertions shall be construed as reducing identity or homology. Methods and computer programs for the alignment are well known.

The term “antibody”, broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. In a full-length antibody, each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

In certain embodiments, the binding molecules of the invention comprise or consist of at least one single domain antibody wherein said domain is a V_{H} immunoglobulin domain. Thus, the binding molecules of the invention comprise or consist of at least one immunoglobulin single variable heavy chain domain antibody (sVD, sdAb or ISV) that has a V_{H} domain, but is devoid of a V_{L} domain. Single domain antibodies have been described in the art; they are antibodies whose complementary determining regions are part of a single domain polypeptide, for example a V_{H} polypeptide.

As described further herein, the binding molecule may comprise two or more V_{H} domains. Such binding molecules may be monospecific or multispecific.

Binding molecules that comprise a single domain antibody wherein said domain is a V_{H} domain are also termed Humabody® V_{H}.

Thus, in some embodiments the binding molecule does not comprise a light chain. In some embodiments the binding molecule does not comprise heavy chain domains C_{H}2 and C_{H}3. In some embodiments the binding molecule does not comprise a hinge region and heavy chain domains C_{H}2 and C_{H}3. In some embodiments the binding molecule does not comprise heavy chain domains C_{H}1, C_{H}2, and CH3. In some embodiments the binding molecule does not comprise heavy chain domain C_{H}1, a hinge region heavy chain domain C_{H}2 and heavy chain domain C_{H}3. In preferred embodiments the binding molecule does not comprise a light chain, a heavy chain domain C_{H}1, a hinge region heavy chain domain C_{H}2 and heavy chain domain C_{H}3.

Each V_{H} domain comprises three CDRs and four FRs, arranged from amino-terminus to carboxy-terminus in the following order: FR1, CDR1, FR2, CDR2, FR3, CDR3, FR4. For example, the V_{H} domain may comprise C or N terminal extensions. In one embodiment, the V_{H} domain comprises C terminal extensions of from 1 to 10, for example 1, 2, 3, 4, 5, 6, 7, 8, 9 or 10 additional amino acids. In one embodiment, the V_{H} domain comprises C terminal extensions of from 1 to 12, for example 1, 2, 3, 4, 5, 6, 7, 8, 9 or 10 additional amino acids of the C_{H}1 domain. In one embodiment, said extension comprises at least 1 alanine residue, for example a single alanine residue, a pair of alanine residues or a triplet of alanine residues. Such extended V_{H} domains are within the scope of the invention. Also within the scope of the invention are V_{H} domains that comprise additional C or N terminal residues, for example linker residues introduced from the expression vector used or His tags, e.g. hexa-His (HHHHHH, SEQ ID NO: 2605).

Preferably, the one or more V_{H} domain is a human V_{H} domain. As used herein, a human V_{H} domain includes a V_{H} domain that is derived from or based on a human V_{H} domain amino acid or nucleic acid sequence. Thus, the term includes variable heavy chain regions derived from human germline immunoglobulin sequences. As used herein, the term human V_{H} domain includes V_{H} domains that are isolated from transgenic mice expressing human immunoglobulin V genes, in particular in response to an immunisation with an antigen of interest. The human V_{H} domains of the invention may include amino acid residues not encoded by human germline immunoglobulin sequences (e.g., mutations introduced in vitro, e.g. by random or site-specific mutagenesis, or introduced by somatic mutation in vivo). The term “human V_{H} domain” therefore also includes modified human V_{H} sequences.

Thus, the invention provides a binding molecule comprising at least one immunoglobulin single domain antibody capable of binding/directed against IL-17RA wherein said domain is a human V_{H} domain and wherein said IL-17A binding molecule comprises at least one antigen binding site. The single domain antibody is specifically directed against/capable of binding human IL-17RA.

As used herein, the term V_{H} or “variable domain” refers to immunoglobulin variable domains defined by Kabat et al., Sequences of Immunological Interest, 5^{th} ed., U.S. Dept. Health & Human Services, Washington, D.C. (1991). The numbering and positioning of CDR amino acid residues within the variable domains is in accordance with the well-known Kabat numbering convention.

More particularly, the invention provides a V_{H} immunoglobulin domain that can bind to human IL-17RA with an affinity, a Kon-rate, a Koff rate, KD and/or KA as further described herein.

The binding molecules of the invention comprise amino acid sequences and preferred sequences and/or parts thereof, such as CDRs, as defined herein.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs have been defined differently according to different systems. The system described by Kabat as used herein. The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and Kabat, et al. (1991) Sequences of Proteins of Immunological Interest, Fifth Edition, U.S. Department of Health and Human Services, NIH Publication No. 91-3242).

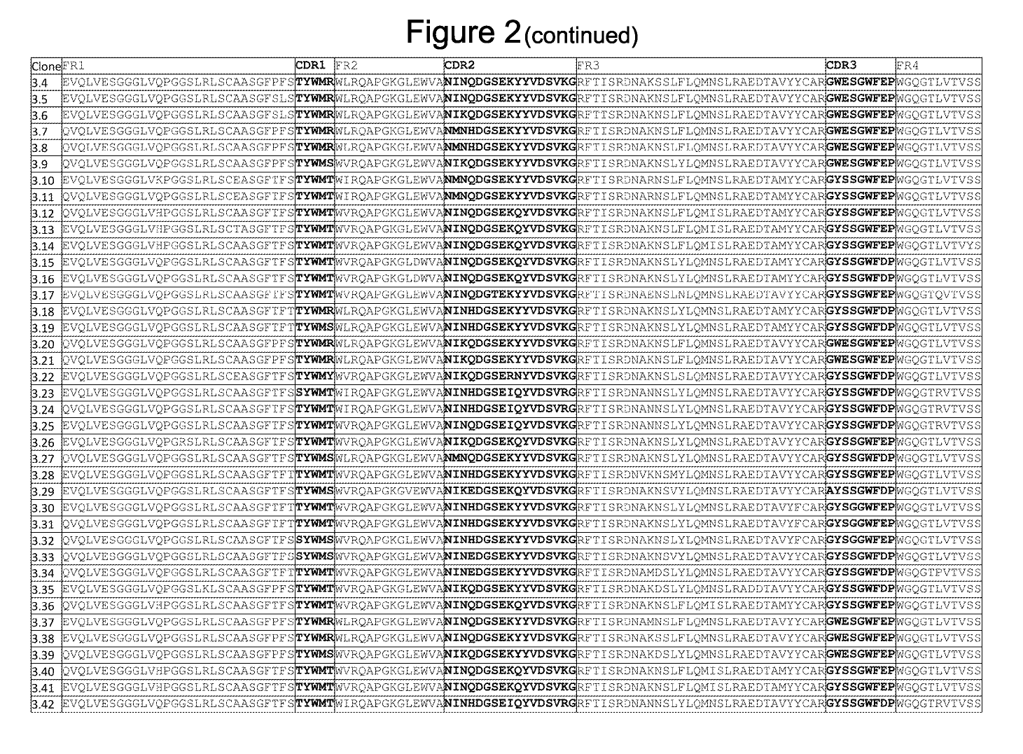

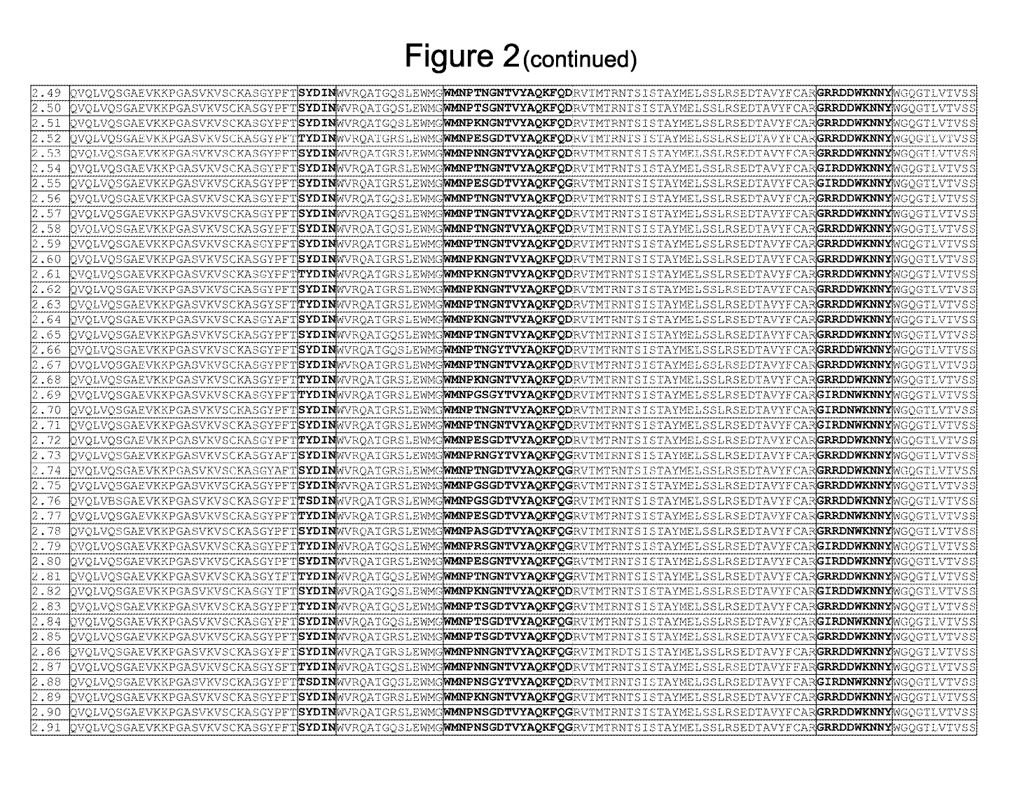

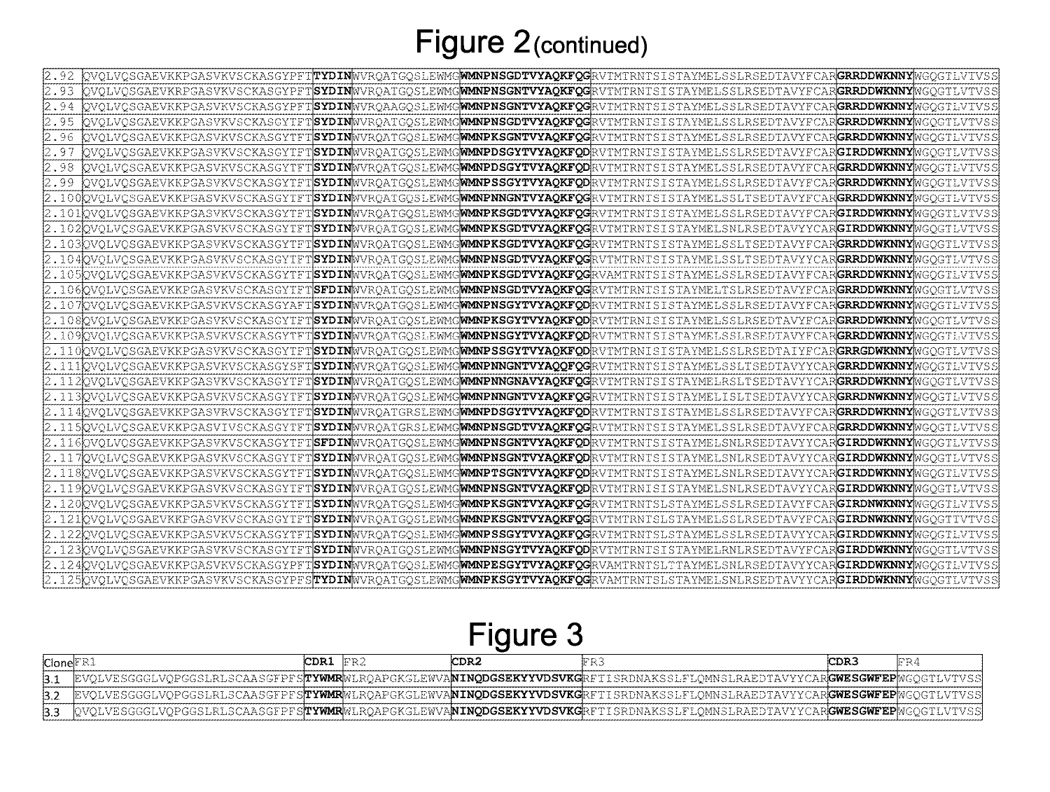

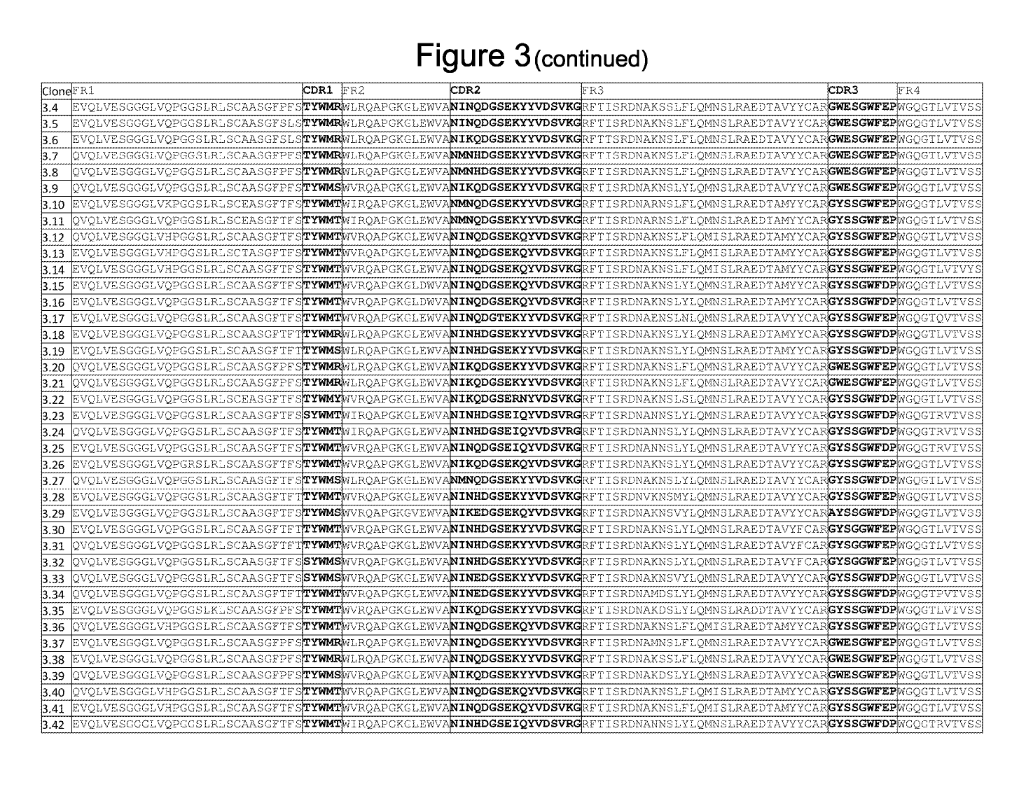

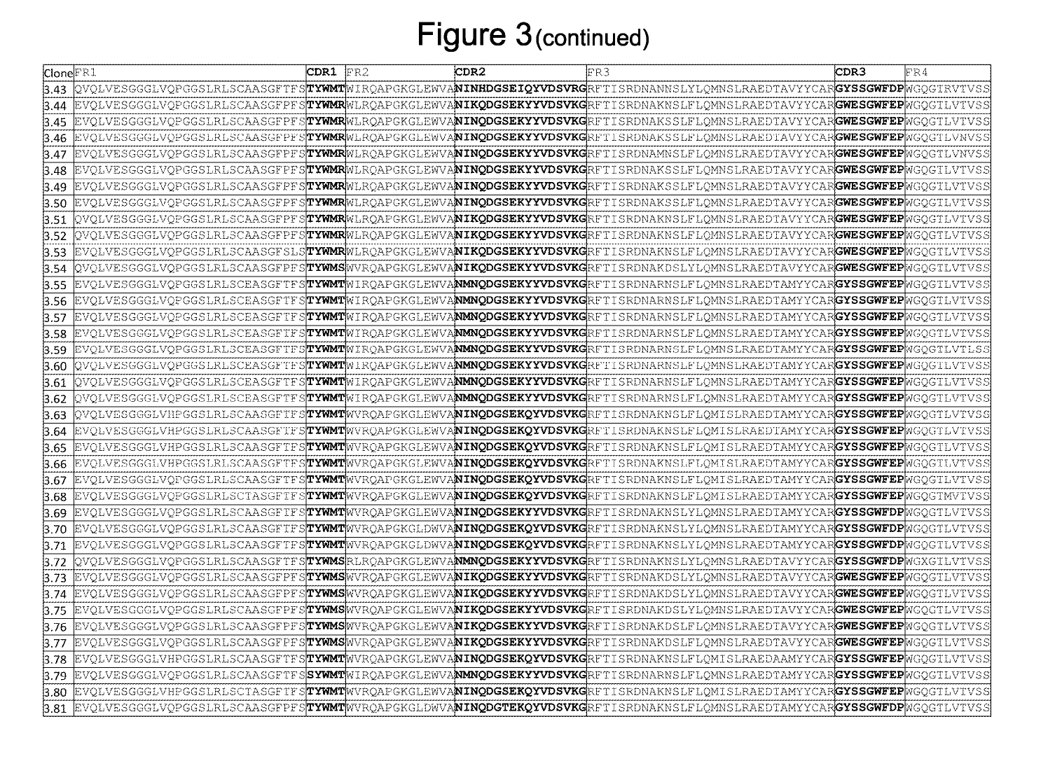

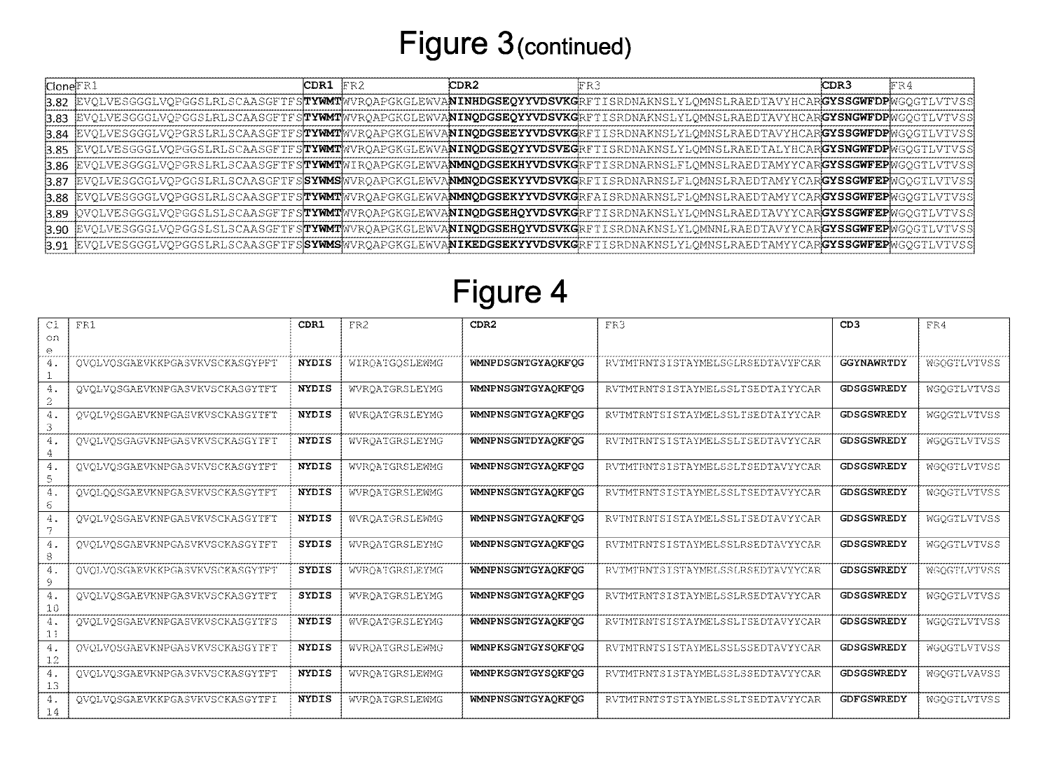

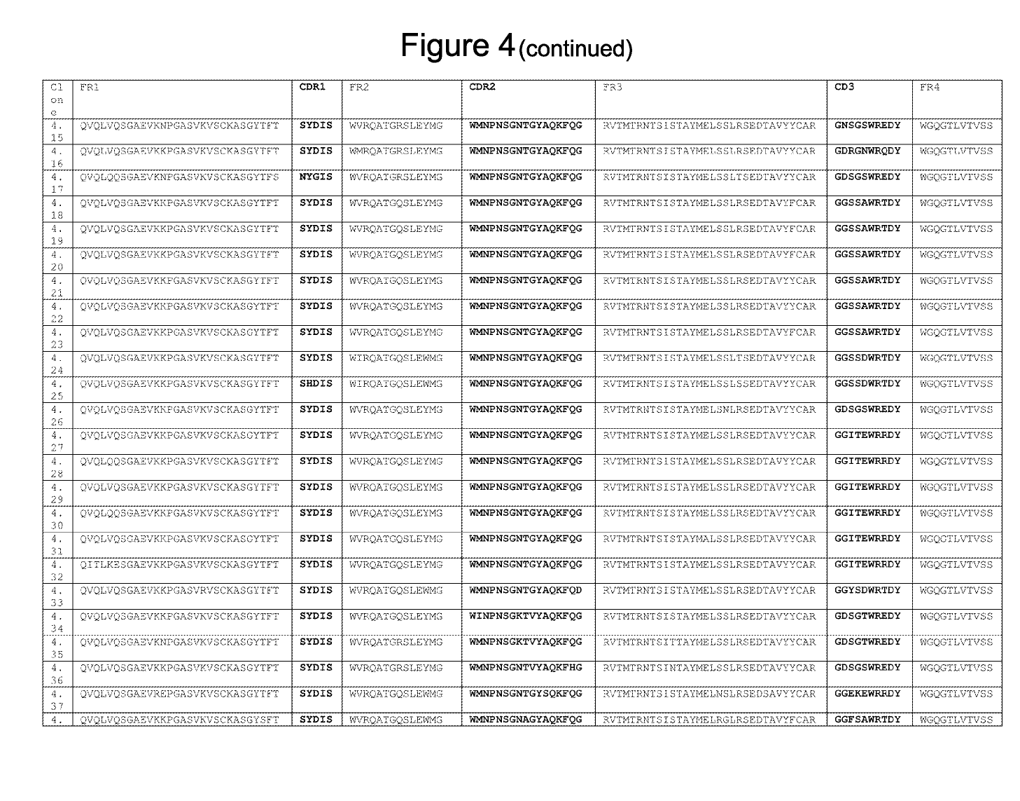

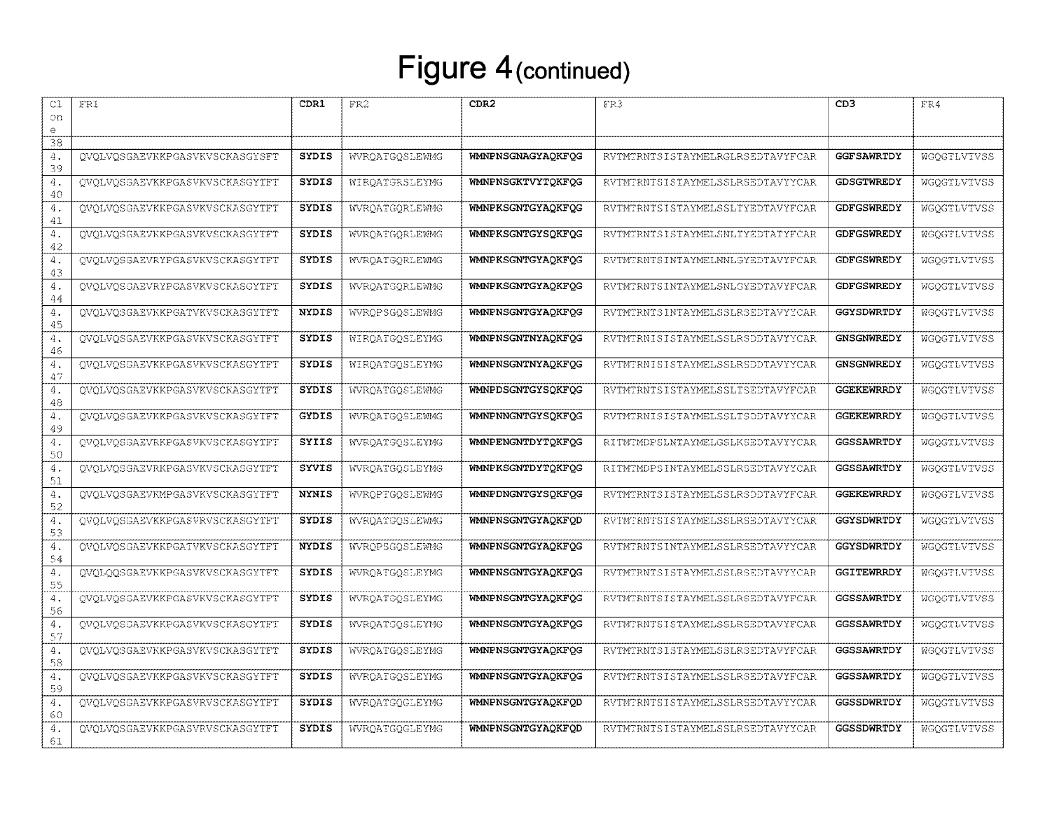

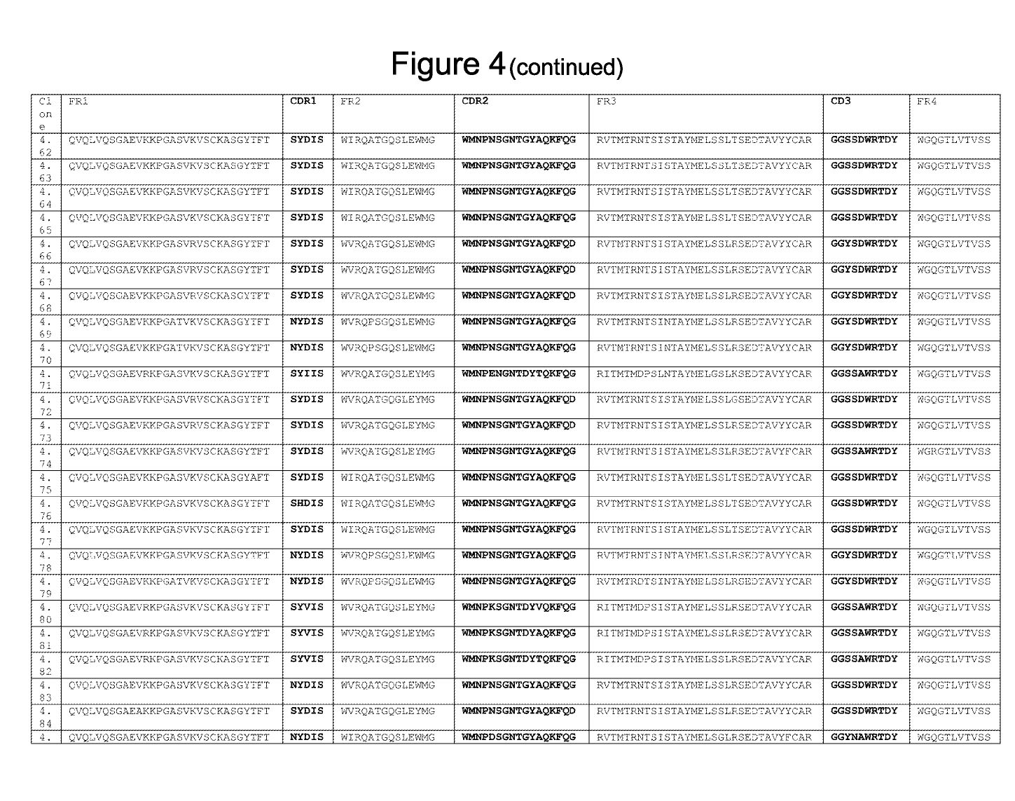

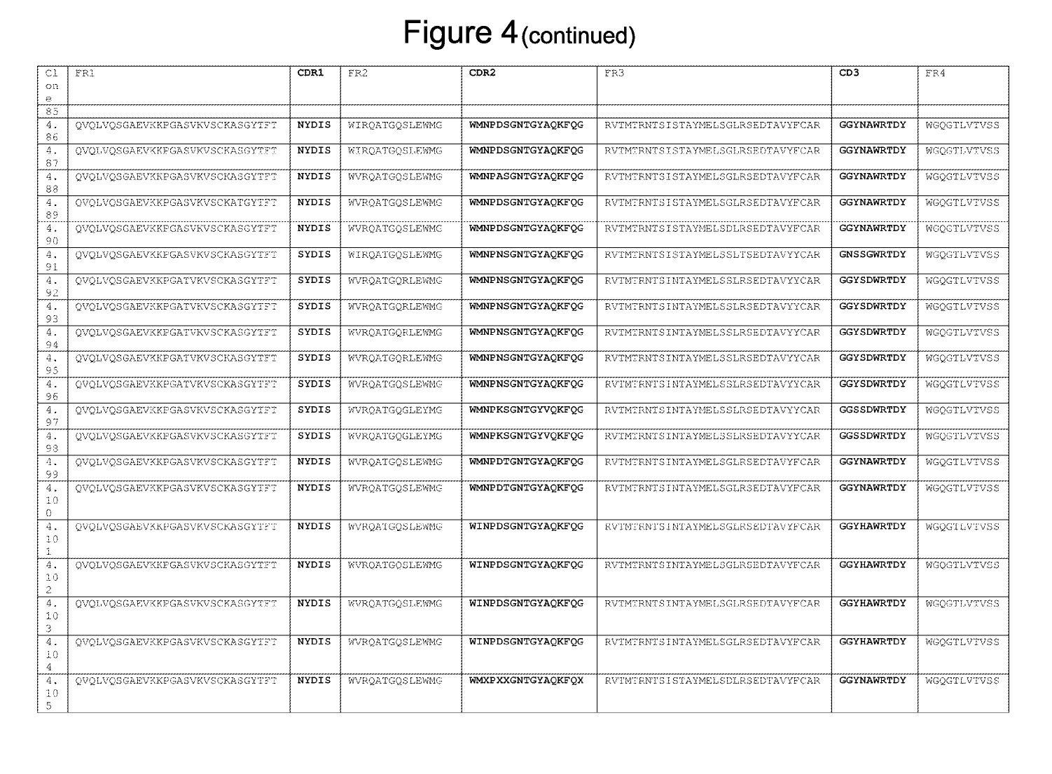

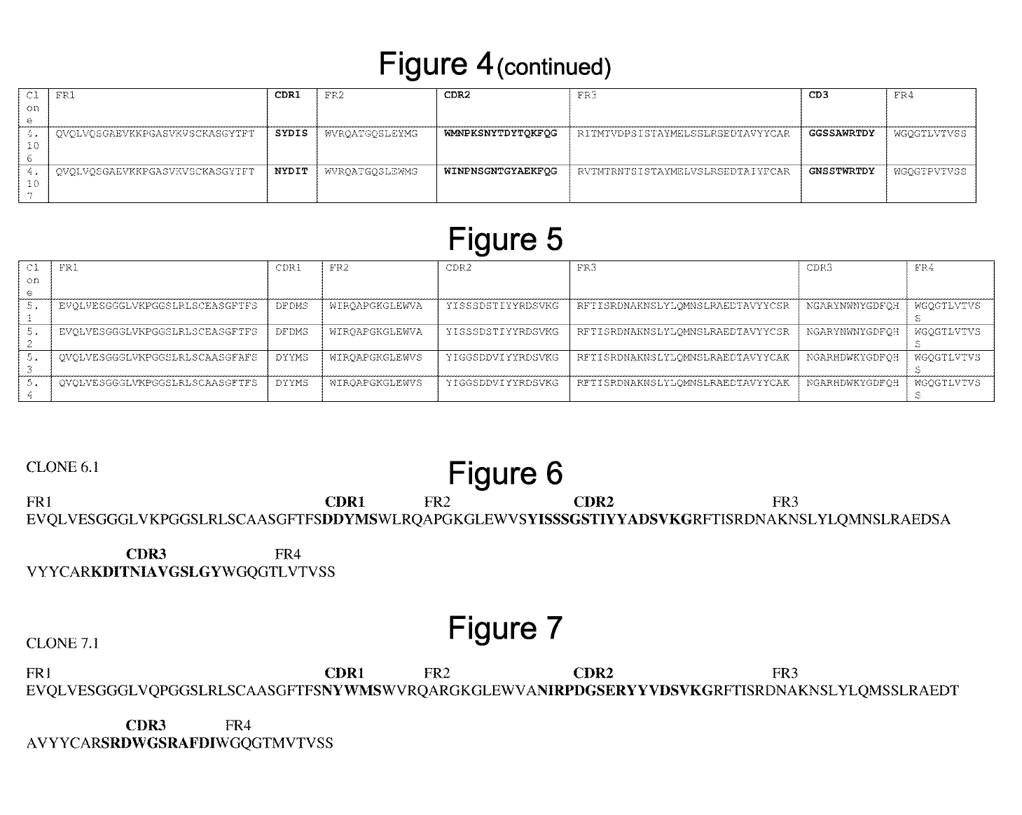

As described in more detail in the experimental part, the inventors isolated parent molecules (resulting in 7 families of clones: done 1.1 is the parent done for family 1 as shown in FIG. 1, clone 2.1 is the parent clone for family 2 as shown in FIG. 2, done 3.1 is the parent clone for family 3 as shown in FIG. 3, done 4.1 is the parent done for family 4 as shown in FIG. 4, done 5.1 is the parent done for family 5 as shown in FIG. 5, clone 6.1 is the parent clone for family 6 as shown in FIG. 6 and done 7.1 is the parent clone for family 7 as shown in FIG. 7); each having a set of CDR sequences (CDR1, 2 and 3) as shown in FIGS. 1, 2, 3, 4, 5, 6 and 7. Through a process of optimization, a panel of clones with CDR3 sequences derived from the parent CDR3 sequences was generated for each of family 1, 2, 3, 4, 5, 6 and 7. Optimised V_{H} domain sequences show improved affinities to IL-17RA and improved potencies in the IL-17RA cell-based assay compared to the parent molecule as shown in the examples.

In one aspect, the invention relates to a binding molecule capable of binding human IL-17RA comprising a human V_{H} domain wherein said V_{H} domain comprises a family 1 or family 1-like sequence.

In one embodiment, the binding molecule comprises or consists of at least one immunoglobulin single domain antibody capable of binding/directed against IL-17RA, preferably human IL-17RA, wherein said domain is a human V_{H} domain and wherein said V_{H} domain comprises a family 1 or family 1-like sequence. These include the V_{H} sequence of the parent clone (clone 1.1) or a part thereof, for example a CDR3 sequence and V_{H} sequences of clones or parts thereof that are derived from the parent clone 1.1 through a process of optimization, for example sequences as shown as shown in FIG. 1. CDR sequences and full length sequences of clones in family 1 are numbered according to table 1 as shown below.

TABLE 1
This shows SEQ ID NOs. of family 1 CDR sequences and of family
1 full-length V_{H} sequences that are within the scope of the
invention. Corresponding sequences are shown in FIG. 1.

| Clone | | | | VH FULL LENGTH |
|---|---|---|---|---|
| name | CDR1 SEQ ID NO. | CDR2 SEQ ID NO. | CDR3 SEQ ID NO. | SEQ ID NO. |
| 1.1 | SEQ ID NO. 1 | SEQ ID NO. 2 | SEQ ID NO. 3 | SEQ ID NO. 4 |
| 1.2 | SEQ ID NO. 5 | SEQ ID NO. 6 | SEQ ID NO. 7 | SEQ ID NO. 8 |
| 1.3 | SEQ ID NO. 9 | SEQ ID NO. 10 | SEQ ID NO. 11 | SEQ ID NO. 12 |
| 1.4 | SEQ ID NO. 13 | SEQ ID NO. 14 | SEQ ID NO. 15 | SEQ ID NO. 16 |
| 1.5 | SEQ ID NO. 17 | SEQ ID NO. 18 | SEQ ID NO. 19 | SEQ ID NO. 20 |
| 1.6 | SEQ ID NO. 21 | SEQ ID NO. 22 | SEQ ID NO. 23 | SEQ ID NO. 24 |
| 1.7 | SEQ ID NO. 25 | SEQ ID NO. 26 | SEQ ID NO. 27 | SEQ ID NO. 28 |
| 1.8 | SEQ ID NO. 29 | SEQ ID NO. 30 | SEQ ID NO. 31 | SEQ ID NO. 32 |
| 1.9 | SEQ ID NO. 33 | SEQ ID NO. 34 | SEQ ID NO. 35 | SEQ ID NO. 36 |
| 1.10 | SEQ ID NO. 37 | SEQ ID NO. 38 | SEQ ID NO. 39 | SEQ ID NO. 40 |
| 1.11 | SEQ ID NO. 41 | SEQ ID NO. 42 | SEQ ID NO. 43 | SEQ ID NO. 44 |
| 1.12 | SEQ ID NO. 45 | SEQ ID NO. 46 | SEQ ID NO. 47 | SEQ ID NO. 48 |
| 1.13 | SEQ ID NO. 49 | SEQ ID NO. 50 | SEQ ID NO. 51 | SEQ ID NO. 52 |
| 1.14 | SEQ ID NO. 53 | SEQ ID NO. 54 | SEQ ID NO. 55 | SEQ ID NO. 56 |
| 1.15 | SEQ ID NO. 57 | SEQ ID NO. 58 | SEQ ID NO. 59 | SEQ ID NO. 60 |
| 1.16 | SEQ ID NO. 61 | SEQ ID NO. 62 | SEQ ID NO. 63 | SEQ ID NO. 64 |
| 1.17 | SEQ ID NO. 65 | SEQ ID NO. 66 | SEQ ID NO. 67 | SEQ ID NO. 68 |
| 1.18 | SEQ ID NO. 69 | SEQ ID NO. 70 | SEQ ID NO. 71 | SEQ ID NO. 72 |
| 1.19 | SEQ ID NO. 73 | SEQ ID NO. 74 | SEQ ID NO. 75 | SEQ ID NO. 76 |
| 1.20 | SEQ ID NO. 77 | SEQ ID NO. 78 | SEQ ID NO. 79 | SEQ ID NO. 80 |
| 1.21 | SEQ ID NO. 81 | SEQ ID NO. 82 | SEQ ID NO. 83 | SEQ ID NO. 84 |
| 1.22 | SEQ ID NO. 85 | SEQ ID NO. 86 | SEQ ID NO. 87 | SEQ ID NO. 88 |
| 1.23 | SEQ ID NO. 89 | SEQ ID NO. 90 | SEQ ID NO. 91 | SEQ ID NO. 92 |
| 1.24 | SEQ ID NO. 93 | SEQ ID NO. 94 | SEQ ID NO. 95 | SEQ ID NO. 96 |
| 1.25 | SEQ ID NO. 97 | SEQ ID NO. 98 | SEQ ID NO. 99 | SEQ ID NO. 100 |
| 1.26 | SEQ ID NO. 101 | SEQ ID NO. 102 | SEQ ID NO. 103 | SEQ ID NO. 104 |
| 1.27 | SEQ ID NO. 105 | SEQ ID NO. 106 | SEQ ID NO. 107 | SEQ ID NO. 108 |
| 1.28 | SEQ ID NO. 109 | SEQ ID NO. 110 | SEQ ID NO. 111 | SEQ ID NO. 112 |
| 1.29 | SEQ ID NO. 113 | SEQ ID NO. 114 | SEQ ID NO. 115 | SEQ ID NO. 116 |
| 1.30 | SEQ ID NO. 117 | SEQ ID NO. 118 | SEQ ID NO. 119 | SEQ ID NO. 120 |
| 1.31 | SEQ ID NO. 121 | SEQ ID NO. 122 | SEQ ID NO. 123 | SEQ ID NO. 124 |
| 1.32 | SEQ ID NO. 125 | SEQ ID NO. 126 | SEQ ID NO. 127 | SEQ ID NO. 128 |
| 1.33 | SEQ ID NO. 129 | SEQ ID NO. 130 | SEQ ID NO. 131 | SEQ ID NO. 132 |
| 1.34 | SEQ ID NO. 133 | SEQ ID NO. 134 | SEQ ID NO. 135 | SEQ ID NO. 136 |
| 1.35 | SEQ ID NO. 137 | SEQ ID NO. 138 | SEQ ID NO. 139 | SEQ ID NO. 140 |
| 1.36 | SEQ ID NO. 141 | SEQ ID NO. 142 | SEQ ID NO. 143 | SEQ ID NO. 144 |
| 1.37 | SEQ ID NO. 145 | SEQ ID NO. 146 | SEQ ID NO. 147 | SEQ ID NO. 148 |
| 1.38 | SEQ ID NO. 149 | SEQ ID NO. 150 | SEQ ID NO. 151 | SEQ ID NO. 152 |
| 1.39 | SEQ ID NO. 153 | SEQ ID NO. 154 | SEQ ID NO. 155 | SEQ ID NO. 156 |
| 1.40 | SEQ ID NO. 157 | SEQ ID NO. 158 | SEQ ID NO. 159 | SEQ ID NO. 160 |
| 1.41 | SEQ ID NO. 161 | SEQ ID NO. 162 | SEQ ID NO. 163 | SEQ ID NO. 164 |
| 1.42 | SEQ ID NO. 165 | SEQ ID NO. 166 | SEQ ID NO. 167 | SEQ ID NO. 168 |
| 1.43 | SEQ ID NO. 169 | SEQ ID NO. 170 | SEQ ID NO. 171 | SEQ ID NO. 172 |
| 1.44 | SEQ ID NO. 173 | SEQ ID NO. 174 | SEQ ID NO. 175 | SEQ ID NO. 176 |
| 1.45 | SEQ ID NO. 177 | SEQ ID NO. 178 | SEQ ID NO. 179 | SEQ ID NO. 180 |
| 1.46 | SEQ ID NO. 181 | SEQ ID NO. 182 | SEQ ID NO. 183 | SEQ ID NO. 184 |
| 1.47 | SEQ ID NO. 185 | SEQ ID NO. 186 | SEQ ID NO. 187 | SEQ ID NO. 188 |
| 1.48 | SEQ ID NO. 189 | SEQ ID NO. 190 | SEQ ID NO. 191 | SEQ ID NO. 192 |
| 1.49 | SEQ ID NO. 193 | SEQ ID NO. 194 | SEQ ID NO. 195 | SEQ ID NO. 196 |
| 1.50 | SEQ ID NO. 197 | SEQ ID NO. 198 | SEQ ID NO. 199 | SEQ ID NO. 200 |
| 1.51 | SEQ ID NO. 201 | SEQ ID NO. 202 | SEQ ID NO. 203 | SEQ ID NO. 204 |
| 1.52 | SEQ ID NO. 205 | SEQ ID NO. 206 | SEQ ID NO. 207 | SEQ ID NO. 208 |
| 1.53 | SEQ ID NO. 209 | SEQ ID NO. 210 | SEQ ID NO. 211 | SEQ ID NO. 212 |
| 1.54 | SEQ ID NO. 213 | SEQ ID NO. 214 | SEQ ID NO. 215 | SEQ ID NO. 216 |
| 1.55 | SEQ ID NO. 217 | SEQ ID NO. 218 | SEQ ID NO. 219 | SEQ ID NO. 220 |
| 1.56 | SEQ ID NO. 221 | SEQ ID NO. 222 | SEQ ID NO. 223 | SEQ ID NO. 224 |
| 1.57 | SEQ ID NO. 225 | SEQ ID NO. 226 | SEQ ID NO. 227 | SEQ ID NO. 228 |
| 1.58 | SEQ ID NO. 229 | SEQ ID NO. 230 | SEQ ID NO. 231 | SEQ ID NO. 232 |
| 1.59 | SEQ ID NO. 233 | SEQ ID NO. 234 | SEQ ID NO. 235 | SEQ ID NO. 236 |
| 1.60 | SEQ ID NO. 237 | SEQ ID NO. 238 | SEQ ID NO. 239 | SEQ ID NO. 240 |
| 1.61 | SEQ ID NO. 241 | SEQ ID NO. 242 | SEQ ID NO. 243 | SEQ ID NO. 244 |
| 1.62 | SEQ ID NO. 245 | SEQ ID NO. 246 | SEQ ID NO. 247 | SEQ ID NO. 248 |
| 1.63 | SEQ ID NO. 249 | SEQ ID NO. 250 | SEQ ID NO. 251 | SEQ ID NO. 252 |
| 1.64 | SEQ ID NO. 253 | SEQ ID NO. 254 | SEQ ID NO. 255 | SEQ ID NO. 256 |
| 1.65 | SEQ ID NO. 257 | SEQ ID NO. 258 | SEQ ID NO. 259 | SEQ ID NO. 260 |
| 1.66 | SEQ ID NO. 261 | SEQ ID NO. 262 | SEQ ID NO. 263 | SEQ ID NO. 264 |
| 1.67 | SEQ ID NO. 265 | SEQ ID NO. 266 | SEQ ID NO. 267 | SEQ ID NO. 268 |
| 1.68 | SEQ ID NO. 269 | SEQ ID NO. 270 | SEQ ID NO. 271 | SEQ ID NO. 272 |
| 1.69 | SEQ ID NO. 273 | SEQ ID NO. 274 | SEQ ID NO. 275 | SEQ ID NO. 276 |
| 1.70 | SEQ ID NO. 277 | SEQ ID NO. 278 | SEQ ID NO. 279 | SEQ ID NO. 280 |
| 1.71 | SEQ ID NO. 281 | SEQ ID NO. 282 | SEQ ID NO. 283 | SEQ ID NO. 284 |
| 1.72 | SEQ ID NO. 285 | SEQ ID NO. 286 | SEQ ID NO. 287 | SEQ ID NO. 288 |
| 1.73 | SEQ ID NO. 289 | SEQ ID NO. 290 | SEQ ID NO. 291 | SEQ ID NO. 292 |
| 1.74 | SEQ ID NO. 293 | SEQ ID NO. 294 | SEQ ID NO. 295 | SEQ ID NO. 296 |
| 1.75 | SEQ ID NO. 297 | SEQ ID NO. 298 | SEQ ID NO. 299 | SEQ ID NO. 300 |
| 1.76 | SEQ ID NO. 301 | SEQ ID NO. 302 | SEQ ID NO. 303 | SEQ ID NO. 304 |
| 1.77 | SEQ ID NO. 305 | SEQ ID NO. 306 | SEQ ID NO. 307 | SEQ ID NO. 308 |
| 1.78 | SEQ ID NO. 309 | SEQ ID NO. 310 | SEQ ID NO. 311 | SEQ ID NO. 312 |
| 1.79 | SEQ ID NO. 313 | SEQ ID NO. 314 | SEQ ID NO. 315 | SEQ ID NO. 316 |
| 1.80 | SEQ ID NO. 317 | SEQ ID NO. 318 | SEQ ID NO. 319 | SEQ ID NO. 320 |
| 1.81 | SEQ ID NO. 321 | SEQ ID NO. 322 | SEQ ID NO. 323 | SEQ ID NO. 324 |
| 1.82 | SEQ ID NO. 325 | SEQ ID NO. 326 | SEQ ID NO. 327 | SEQ ID NO. 328 |
| 1.83 | SEQ ID NO. 329 | SEQ ID NO. 330 | SEQ ID NO. 331 | SEQ ID NO. 332 |
| 1.84 | SEQ ID NO. 333 | SEQ ID NO. 334 | SEQ ID NO. 335 | SEQ ID NO. 336 |
| 1.85 | SEQ ID NO. 337 | SEQ ID NO. 338 | SEQ ID NO. 339 | SEQ ID NO. 340 |
| 1.86 | SEQ ID NO. 341 | SEQ ID NO. 342 | SEQ ID NO. 343 | SEQ ID NO. 344 |
| 1.87 | SEQ ID NO. 345 | SEQ ID NO. 346 | SEQ ID NO. 347 | SEQ ID NO. 348 |
| 1.88 | SEQ ID NO. 349 | SEQ ID NO. 350 | SEQ ID NO. 351 | SEQ ID NO. 352 |
| 1.89 | SEQ ID NO. 353 | SEQ ID NO. 354 | SEQ ID NO. 355 | SEQ ID NO. 356 |
| 1.90 | SEQ ID NO. 357 | SEQ ID NO. 358 | SEQ ID NO. 359 | SEQ ID NO. 360 |
| 1.91 | SEQ ID NO. 361 | SEQ ID NO. 362 | SEQ ID NO. 363 | SEQ ID NO. 364 |
| 1.92 | SEQ ID NO. 365 | SEQ ID NO. 366 | SEQ ID NO. 367 | SEQ ID NO. 368 |
| 1.93 | SEQ ID NO. 369 | SEQ ID NO. 370 | SEQ ID NO. 371 | SEQ ID NO. 372 |
| 1.94 | SEQ ID NO. 373 | SEQ ID NO. 374 | SEQ ID NO. 375 | SEQ ID NO. 376 |
| 1.95 | SEQ ID NO. 377 | SEQ ID NO. 378 | SEQ ID NO. 379 | SEQ ID NO. 380 |
| 1.96 | SEQ ID NO. 381 | SEQ ID NO. 382 | SEQ ID NO. 383 | SEQ ID NO. 384 |
| 1.97 | SEQ ID NO. 385 | SEQ ID NO. 386 | SEQ ID NO. 387 | SEQ ID NO. 388 |
| 1.98 | SEQ ID NO. 389 | SEQ ID NO. 390 | SEQ ID NO. 391 | SEQ ID NO. 392 |
| 1.99 | SEQ ID NO. 393 | SEQ ID NO. 394 | SEQ ID NO. 395 | SEQ ID NO. 396 |
| 1.100 | SEQ ID NO. 397 | SEQ ID NO. 398 | SEQ ID NO. 399 | SEQ ID NO. 400 |
| 1.101 | SEQ ID NO. 401 | SEQ ID NO. 402 | SEQ ID NO. 403 | SEQ ID NO. 404 |
| 1.102 | SEQ ID NO. 405 | SEQ ID NO. 406 | SEQ ID NO. 407 | SEQ ID NO. 408 |
| 1.103 | SEQ ID NO. 409 | SEQ ID NO. 410 | SEQ ID NO. 411 | SEQ ID NO. 412 |
| 1.104 | SEQ ID NO. 413 | SEQ ID NO. 414 | SEQ ID NO. 415 | SEQ ID NO. 416 |
| 1.105 | SEQ ID NO. 417 | SEQ ID NO. 418 | SEQ ID NO. 419 | SEQ ID NO. 420 |
| 1.106 | SEQ ID NO. 421 | SEQ ID NO. 422 | SEQ ID NO. 423 | SEQ ID NO. 424 |
| 1.107 | SEQ ID NO. 425 | SEQ ID NO. 426 | SEQ ID NO. 427 | SEQ ID NO. 428 |
| 1.108 | SEQ ID NO. 429 | SEQ ID NO. 430 | SEQ ID NO. 431 | SEQ ID NO. 432 |
| 1.109 | SEQ ID NO. 433 | SEQ ID NO. 434 | SEQ ID NO. 435 | SEQ ID NO. 436 |
| 1.110 | SEQ ID NO. 437 | SEQ ID NO. 438 | SEQ ID NO. 439 | SEQ ID NO. 440 |
| 1.111 | SEQ ID NO. 441 | SEQ ID NO. 442 | SEQ ID NO. 443 | SEQ ID NO. 444 |
| 1.112 | SEQ ID NO. 445 | SEQ ID NO. 446 | SEQ ID NO. 447 | SEQ ID NO. 448 |
| 1.113 | SEQ ID NO. 449 | SEQ ID NO. 450 | SEQ ID NO. 451 | SEQ ID NO. 452 |
| 1.114 | SEQ ID NO. 453 | SEQ ID NO. 454 | SEQ ID NO. 455 | SEQ ID NO. 456 |
| 1.115 | SEQ ID NO. 457 | SEQ ID NO. 458 | SEQ ID NO. 459 | SEQ ID NO. 460 |
| 1.116 | SEQ ID NO. 461 | SEQ ID NO. 462 | SEQ ID NO. 463 | SEQ ID NO. 464 |
| 1.117 | SEQ ID NO. 465 | SEQ ID NO. 466 | SEQ ID NO. 467 | SEQ ID NO. 468 |
| 1.118 | SEQ ID NO. 469 | SEQ ID NO. 470 | SEQ ID NO. 471 | SEQ ID NO. 472 |
| 1.119 | SEQ ID NO. 473 | SEQ ID NO. 474 | SEQ ID NO. 475 | SEQ ID NO. 476 |
| 1.120 | SEQ ID NO. 477 | SEQ ID NO. 478 | SEQ ID NO. 479 | SEQ ID NO. 480 |
| 1.121 | SEQ ID NO. 481 | SEQ ID NO. 482 | SEQ ID NO. 483 | SEQ ID NO. 484 |
| 1.122 | SEQ ID NO. 485 | SEQ ID NO. 486 | SEQ ID NO. 487 | SEQ ID NO. 488 |
| 1.123 | SEQ ID NO. 489 | SEQ ID NO. 490 | SEQ ID NO. 491 | SEQ ID NO. 492 |
| 1.124 | SEQ ID NO. 493 | SEQ ID NO. 494 | SEQ ID NO. 495 | SEQ ID NO. 496 |
| 1.125 | SEQ ID NO. 497 | SEQ ID NO. 498 | SEQ ID NO. 499 | SEQ ID NO. 500 |
| 1.126 | SEQ ID NO. 501 | SEQ ID NO. 502 | SEQ ID NO. 503 | SEQ ID NO. 504 |
| 1.127 | SEQ ID NO. 505 | SEQ ID NO. 506 | SEQ ID NO. 507 | SEQ ID NO. 508 |
| 1.128 | SEQ ID NO. 509 | SEQ ID NO. 510 | SEQ ID NO. 511 | SEQ ID NO. 512 |
| 1.129 | SEQ ID NO. 513 | SEQ ID NO. 514 | SEQ ID NO. 515 | SEQ ID NO. 516 |
| 1.130 | SEQ ID NO. 517 | SEQ ID NO. 518 | SEQ ID NO. 519 | SEQ ID NO. 520 |
| 1.131 | SEQ ID NO. 521 | SEQ ID NO. 522 | SEQ ID NO. 523 | SEQ ID NO. 524 |
| 1.132 | SEQ ID NO. 525 | SEQ ID NO. 526 | SEQ ID NO. 527 | SEQ ID NO. 528 |
| 1.133 | SEQ ID NO. 529 | SEQ ID NO. 530 | SEQ ID NO. 531 | SEQ ID NO. 532 |
| 1.134 | SEQ ID NO. 533 | SEQ ID NO. 534 | SEQ ID NO. 535 | SEQ ID NO. 536 |
| 1.135 | SEQ ID NO. 537 | SEQ ID NO. 538 | SEQ ID NO. 539 | SEQ ID NO. 540 |
| 1.136 | SEQ ID NO. 541 | SEQ ID NO. 542 | SEQ ID NO. 543 | SEQ ID NO. 544 |
| 1.137 | SEQ ID NO. 545 | SEQ ID NO. 546 | SEQ ID NO. 547 | SEQ ID NO. 548 |
| 1.138 | SEQ ID NO. 549 | SEQ ID NO. 550 | SEQ ID NO. 551 | SEQ ID NO. 552 |
| 1.139 | SEQ ID NO. 553 | SEQ ID NO. 554 | SEQ ID NO. 555 | SEQ ID NO. 556 |
| 1.140 | SEQ ID NO. 557 | SEQ ID NO. 558 | SEQ ID NO. 559 | SEQ ID NO. 560 |
| 1.141 | SEQ ID NO. 561 | SEQ ID NO. 562 | SEQ ID NO. 563 | SEQ ID NO. 564 |
| 1.142 | SEQ ID NO. 565 | SEQ ID NO. 566 | SEQ ID NO. 567 | SEQ ID NO. 568 |
| 1.143 | SEQ ID NO. 569 | SEQ ID NO. 570 | SEQ ID NO. 571 | SEQ ID NO. 572 |
| 1.144 | SEQ ID NO. 573 | SEQ ID NO. 574 | SEQ ID NO. 575 | SEQ ID NO. 576 |
| 1.145 | SEQ ID NO. 577 | SEQ ID NO. 578 | SEQ ID NO. 579 | SEQ ID NO. 580 |
| 1.146 | SEQ ID NO. 581 | SEQ ID NO. 582 | SEQ ID NO. 583 | SEQ ID NO. 584 |
| 1.147 | SEQ ID NO. 585 | SEQ ID NO. 586 | SEQ ID NO. 587 | SEQ ID NO. 588 |
| 1.148 | SEQ ID NO. 589 | SEQ ID NO. 590 | SEQ ID NO. 591 | SEQ ID NO. 592 |
| 1.149 | SEQ ID NO. 593 | SEQ ID NO. 594 | SEQ ID NO. 595 | SEQ ID NO. 596 |
| 1.150 | SEQ ID NO. 597 | SEQ ID NO. 598 | SEQ ID NO. 599 | SEQ ID NO. 600 |
| 1.151 | SEQ ID NO. 601 | SEQ ID NO. 602 | SEQ ID NO. 603 | SEQ ID NO. 604 |
| 1.152 | SEQ ID NO. 605 | SEQ ID NO. 606 | SEQ ID NO. 607 | SEQ ID NO. 608 |
| 1.153 | SEQ ID NO. 609 | SEQ ID NO. 610 | SEQ ID NO. 611 | SEQ ID NO. 612 |
| 1.154 | SEQ ID NO. 613 | SEQ ID NO. 614 | SEQ ID NO. 615 | SEQ ID NO. 616 |
| 1.155 | SEQ ID NO. 617 | SEQ ID NO. 618 | SEQ ID NO. 619 | SEQ ID NO. 620 |
| 1.156 | SEQ ID NO. 621 | SEQ ID NO. 622 | SEQ ID NO. 623 | SEQ ID NO. 624 |
| 1.157 | SEQ ID NO. 625 | SEQ ID NO. 626 | SEQ ID NO. 627 | SEQ ID NO. 628 |
| 1.158 | SEQ ID NO. 629 | SEQ ID NO. 630 | SEQ ID NO. 631 | SEQ ID NO. 632 |
| 1.159 | SEQ ID NO. 633 | SEQ ID NO. 634 | SEQ ID NO. 635 | SEQ ID NO. 636 |
| 1.160 | SEQ ID NO. 637 | SEQ ID NO. 638 | SEQ ID NO. 639 | SEQ ID NO. 640 |
| 1.161 | SEQ ID NO. 641 | SEQ ID NO. 642 | SEQ ID NO. 643 | SEQ ID NO. 644 |
| 1.162 | SEQ ID NO. 645 | SEQ ID NO. 646 | SEQ ID NO. 647 | SEQ ID NO. 648 |
| 1.163 | SEQ ID NO. 649 | SEQ ID NO. 650 | SEQ ID NO. 651 | SEQ ID NO. 652 |
| 1.164 | SEQ ID NO. 653 | SEQ ID NO. 654 | SEQ ID NO. 655 | SEQ ID NO. 656 |
| 1.165 | SEQ ID NO. 657 | SEQ ID NO. 658 | SEQ ID NO. 659 | SEQ ID NO. 660 |
| 1.166 | SEQ ID NO. 661 | SEQ ID NO. 662 | SEQ ID NO. 663 | SEQ ID NO. 664 |
| 1.167 | SEQ ID NO. 665 | SEQ ID NO. 666 | SEQ ID NO. 667 | SEQ ID NO. 668 |
| 1.168 | SEQ ID NO. 669 | SEQ ID NO. 670 | SEQ ID NO. 671 | SEQ ID NO. 672 |
| 1.169 | SEQ ID NO. 673 | SEQ ID NO. 674 | SEQ ID NO. 675 | SEQ ID NO. 676 |
| 1.170 | SEQ ID NO. 677 | SEQ ID NO. 678 | SEQ ID NO. 679 | SEQ ID NO. 680 |
| 1.171 | SEQ ID NO. 681 | SEQ ID NO. 682 | SEQ ID NO. 683 | SEQ ID NO. 684 |
| 1.172 | SEQ ID NO. 685 | SEQ ID NO. 686 | SEQ ID NO. 687 | SEQ ID NO. 688 |
| 1.173 | SEQ ID NO. 689 | SEQ ID NO. 690 | SEQ ID NO. 691 | SEQ ID NO. 692 |
| 1.174 | SEQ ID NO. 693 | SEQ ID NO. 694 | SEQ ID NO. 695 | SEQ ID NO. 696 |
| 1.175 | SEQ ID NO. 697 | SEQ ID NO. 698 | SEQ ID NO. 699 | SEQ ID NO. 700 |
| 1.176 | SEQ ID NO. 701 | SEQ ID NO. 702 | SEQ ID NO. 703 | SEQ ID NO. 704 |
| 1.177 | SEQ ID NO. 705 | SEQ ID NO. 706 | SEQ ID NO. 707 | SEQ ID NO. 708 |
| 1.178 | SEQ ID NO. 709 | SEQ ID NO. 710 | SEQ ID NO. 711 | SEQ ID NO. 712 |
| 1.179 | SEQ ID NO. 713 | SEQ ID NO. 714 | SEQ ID NO. 715 | SEQ ID NO. 716 |
| 1.180 | SEQ ID NO. 717 | SEQ ID NO. 718 | SEQ ID NO. 719 | SEQ ID NO. 720 |
| 1.181 | SEQ ID NO. 721 | SEQ ID NO. 722 | SEQ ID NO. 723 | SEQ ID NO. 724 |
| 1.182 | SEQ ID NO. 725 | SEQ ID NO. 726 | SEQ ID NO. 727 | SEQ ID NO. 728 |
| 1.183 | SEQ ID NO. 729 | SEQ ID NO. 730 | SEQ ID NO. 731 | SEQ ID NO. 732 |
| 1.184 | SEQ ID NO. 733 | SEQ ID NO. 734 | SEQ ID NO. 735 | SEQ ID NO. 736 |
| 1.185 | SEQ ID NO. 737 | SEQ ID NO. 738 | SEQ ID NO. 739 | SEQ ID NO. 740 |
| 1.186 | SEQ ID NO. 741 | SEQ ID NO. 742 | SEQ ID NO. 743 | SEQ ID NO. 744 |
| 1.187 | SEQ ID NO. 745 | SEQ ID NO. 746 | SEQ ID NO. 747 | SEQ ID NO. 748 |
| 1.188 | SEQ ID NO. 749 | SEQ ID NO. 750 | SEQ ID NO. 751 | SEQ ID NO. 752 |
| 1.189 | SEQ ID NO. 753 | SEQ ID NO. 754 | SEQ ID NO. 755 | SEQ ID NO. 756 |
| 1.190 | SEQ ID NO. 757 | SEQ ID NO. 758 | SEQ ID NO. 759 | SEQ ID NO. 760 |
| 1.191 | SEQ ID NO. 761 | SEQ ID NO. 762 | SEQ ID NO. 763 | SEQ ID NO. 764 |
| 1.192 | SEQ ID NO. 765 | SEQ ID NO. 766 | SEQ ID NO. 767 | SEQ ID NO. 768 |
| 1.193 | SEQ ID NO. 769 | SEQ ID NO. 770 | SEQ ID NO. 771 | SEQ ID NO. 772 |
| 1.194 | SEQ ID NO. 773 | SEQ ID NO. 774 | SEQ ID NO. 775 | SEQ ID NO. 776 |
| 1.195 | SEQ ID NO. 777 | SEQ ID NO. 778 | SEQ ID NO. 779 | SEQ ID NO. 780 |
| 1.196 | SEQ ID NO. 781 | SEQ ID NO. 782 | SEQ ID NO. 783 | SEQ ID NO. 784 |
| 1.197 | SEQ ID NO. 785 | SEQ ID NO. 786 | SEQ ID NO. 787 | SEQ ID NO. 788 |
| 1.198 | SEQ ID NO. 789 | SEQ ID NO. 790 | SEQ ID NO. 791 | SEQ ID NO. 792 |
| 1.199 | SEQ ID NO. 793 | SEQ ID NO. 794 | SEQ ID NO. 795 | SEQ ID NO. 796 |
| 1.200 | SEQ ID NO. 797 | SEQ ID NO. 798 | SEQ ID NO. 799 | SEQ ID NO. 800 |
| 1.201 | SEQ ID NO. 801 | SEQ ID NO. 802 | SEQ ID NO. 803 | SEQ ID NO. 804 |
| 1.202 | SEQ ID NO. 805 | SEQ ID NO. 806 | SEQ ID NO. 807 | SEQ ID NO. 808 |
| 1.203 | SEQ ID NO. 809 | SEQ ID NO. 810 | SEQ ID NO. 811 | SEQ ID NO. 812 |
| 1.204 | SEQ ID NO. 813 | SEQ ID NO. 814 | SEQ ID NO. 815 | SEQ ID NO. 816 |
| 1.205 | SEQ ID NO. 817 | SEQ ID NO. 818 | SEQ ID NO. 819 | SEQ ID NO. 820 |
| 1.206 | SEQ ID NO. 821 | SEQ ID NO. 822 | SEQ ID NO. 823 | SEQ ID NO. 824 |
| 1.207 | SEQ ID NO. 825 | SEQ ID NO. 826 | SEQ ID NO. 827 | SEQ ID NO. 828 |
| 1.208 | SEQ ID NO. 829 | SEQ ID NO. 830 | SEQ ID NO. 831 | SEQ ID NO. 832 |
| 1.209 | SEQ ID NO. 833 | SEQ ID NO. 834 | SEQ ID NO. 835 | SEQ ID NO. 836 |
| 1.210 | SEQ ID NO. 837 | SEQ ID NO. 838 | SEQ ID NO. 839 | SEQ ID NO. 840 |
| 1.211 | SEQ ID NO. 841 | SEQ ID NO. 842 | SEQ ID NO. 843 | SEQ ID NO. 844 |
| 1.212 | SEQ ID NO. 845 | SEQ ID NO. 846 | SEQ ID NO. 847 | SEQ ID NO. 848 |
| 1.213 | SEQ ID NO. 849 | SEQ ID NO. 850 | SEQ ID NO. 851 | SEQ ID NO. 852 |
| 1.214 | SEQ ID NO. 853 | SEQ ID NO. 854 | SEQ ID NO. 855 | SEQ ID NO. 856 |
| 1.215 | SEQ ID NO. 857 | SEQ ID NO. 858 | SEQ ID NO. 859 | SEQ ID NO. 860 |
| 1.216 | SEQ ID NO. 861 | SEQ ID NO. 862 | SEQ ID NO. 863 | SEQ ID NO. 864 |
| 1.217 | SEQ ID NO. 865 | SEQ ID NO. 866 | SEQ ID NO. 867 | SEQ ID NO. 868 |
| 1.218 | SEQ ID NO. 869 | SEQ ID NO. 870 | SEQ ID NO. 871 | SEQ ID NO. 872 |
| 1.219 | SEQ ID NO. 873 | SEQ ID NO. 874 | SEQ ID NO. 875 | SEQ ID NO. 876 |
| 1.220 | SEQ ID NO. 877 | SEQ ID NO. 878 | SEQ ID NO. 879 | SEQ ID NO. 880 |
| 1.221 | SEQ ID NO. 881 | SEQ ID NO. 882 | SEQ ID NO. 883 | SEQ ID NO. 884 |
| 1.222 | SEQ ID NO. 885 | SEQ ID NO. 886 | SEQ ID NO. 887 | SEQ ID NO. 888 |
| 1.223 | SEQ ID NO. 889 | SEQ ID NO. 890 | SEQ ID NO. 891 | SEQ ID NO. 892 |
| 1.224 | SEQ ID NO. 893 | SEQ ID NO. 894 | SEQ ID NO. 895 | SEQ ID NO. 896 |
| 1.225 | SEQ ID NO. 897 | SEQ ID NO. 898 | SEQ ID NO. 899 | SEQ ID NO. 900 |
| 1.226 | SEQ ID NO. 901 | SEQ ID NO. 902 | SEQ ID NO. 903 | SEQ ID NO. 904 |
| 1.227 | SEQ ID NO. 905 | SEQ ID NO. 906 | SEQ ID NO. 907 | SEQ ID NO. 908 |
| 1.228 | SEQ ID NO. 909 | SEQ ID NO. 910 | SEQ ID NO. 911 | SEQ ID NO. 912 |
| 1.229 | SEQ ID NO. 913 | SEQ ID NO. 914 | SEQ ID NO. 915 | SEQ ID NO. 916 |
| 1.230 | SEQ ID NO. 917 | SEQ ID NO. 918 | SEQ ID NO. 919 | SEQ ID NO. 920 |
| 1.231 | SEQ ID NO. 921 | SEQ ID NO. 922 | SEQ ID NO. 923 | SEQ ID NO. 924 |
| 1.232 | SEQ ID NO. 925 | SEQ ID NO. 926 | SEQ ID NO. 927 | SEQ ID NO. 928 |
| 1.233 | SEQ ID NO. 929 | SEQ ID NO. 930 | SEQ ID NO. 931 | SEQ ID NO. 932 |
| 1.234 | SEQ ID NO. 933 | SEQ ID NO. 934 | SEQ ID NO. 935 | SEQ ID NO. 936 |
| 1.235 | SEQ ID NO. 937 | SEQ ID NO. 938 | SEQ ID NO. 939 | SEQ ID NO. 940 |
| 1.236 | SEQ ID NO. 941 | SEQ ID NO. 942 | SEQ ID NO. 943 | SEQ ID NO. 944 |
| 1.237 | SEQ ID NO. 945 | SEQ ID NO. 946 | SEQ ID NO. 947 | SEQ ID NO. 948 |
| 1.238 | SEQ ID NO. 949 | SEQ ID NO. 950 | SEQ ID NO. 951 | SEQ ID NO. 952 |
| 1.239 | SEQ ID NO. 953 | SEQ ID NO. 954 | SEQ ID NO. 955 | SEQ ID NO. 956 |
| 1.240 | SEQ ID NO. 957 | SEQ ID NO. 958 | SEQ ID NO. 959 | SEQ ID NO. 960 |
| 1.241 | SEQ ID NO. 961 | SEQ ID NO. 962 | SEQ ID NO. 963 | SEQ ID NO. 964 |
| 1.242 | SEQ ID NO. 965 | SEQ ID NO. 966 | SEQ ID NO. 967 | SEQ ID NO. 968 |
| 1.243 | SEQ ID NO. 969 | SEQ ID NO. 970 | SEQ ID NO. 971 | SEQ ID NO. 972 |
| 1.244 | SEQ ID NO. 973 | SEQ ID NO. 974 | SEQ ID NO. 975 | SEQ ID NO. 976 |
| 1.245 | SEQ ID NO. 977 | SEQ ID NO. 978 | SEQ ID NO. 979 | SEQ ID NO. 980 |
| 1.246 | SEQ ID NO. 981 | SEQ ID NO. 982 | SEQ ID NO. 983 | SEQ ID NO. 984 |
| 1.247 | SEQ ID NO. 985 | SEQ ID NO. 986 | SEQ ID NO. 987 | SEQ ID NO. 988 |
| 1.248 | SEQ ID NO. 989 | SEQ ID NO. 990 | SEQ ID NO. 991 | SEQ ID NO. 992 |
| 1.249 | SEQ ID NO. 993 | SEQ ID NO. 994 | SEQ ID NO. 995 | SEQ ID NO. 996 |
| 1.250 | SEQ ID NO. 997 | SEQ ID NO. 998 | SEQ ID NO. 999 | SEQ ID NO. 1000 |
| 1.251 | SEQ ID NO. 1001 | SEQ ID NO. 1002 | SEQ ID NO. 1003 | SEQ ID NO. 1004 |
| 1.252 | SEQ ID NO. 1005 | SEQ ID NO. 1006 | SEQ ID NO. 1007 | SEQ ID NO. 1008 |
| 1.253 | SEQ ID NO. 1009 | SEQ ID NO. 1010 | SEQ ID NO. 1011 | SEQ ID NO. 1012 |
| 1.254 | SEQ ID NO. 1013 | SEQ ID NO. 1014 | SEQ ID NO. 1015 | SEQ ID NO. 1016 |
| 1.255 | SEQ ID NO. 1017 | SEQ ID NO. 1018 | SEQ ID NO. 1019 | SEQ ID NO. 1020 |
| 1.256 | SEQ ID NO. 1021 | SEQ ID NO. 1022 | SEQ ID NO. 1023 | SEQ ID NO. 1024 |
| 1.257 | SEQ ID NO. 1025 | SEQ ID NO. 1026 | SEQ ID NO. 1027 | SEQ ID NO. 1028 |
| 1.258 | SEQ ID NO. 1029 | SEQ ID NO. 1030 | SEQ ID NO. 1031 | SEQ ID NO. 1032 |
| 1.259 | SEQ ID NO. 1033 | SEQ ID NO. 1034 | SEQ ID NO. 1035 | SEQ ID NO. 1036 |
| 1.260 | SEQ ID NO. 1037 | SEQ ID NO. 1038 | SEQ ID NO. 1039 | SEQ ID NO. 1040 |
| 1.261 | SEQ ID NO. 1041 | SEQ ID NO. 1042 | SEQ ID NO. 1043 | SEQ ID NO. 1044 |
| 1.262 | SEQ ID NO. 1045 | SEQ ID NO. 1046 | SEQ ID NO. 1047 | SEQ ID NO. 1048 |
| 1.263 | SEQ ID NO. 1049 | SEQ ID NO. 1050 | SEQ ID NO. 1051 | SEQ ID NO. 1052 |
| 1.264 | SEQ ID NO. 1053 | SEQ ID NO. 1054 | SEQ ID NO. 1055 | SEQ ID NO. 1056 |
| 1.265 | SEQ ID NO. 1057 | SEQ ID NO. 1058 | SEQ ID NO. 1059 | SEQ ID NO. 1060 |
| 1.266 | SEQ ID NO. 1061 | SEQ ID NO. 1062 | SEQ ID NO. 1063 | SEQ ID NO. 1064 |
| 1.267 | SEQ ID NO. 1065 | SEQ ID NO. 1066 | SEQ ID NO. 1067 | SEQ ID NO. 1068 |
| 1.268 | SEQ ID NO. 1069 | SEQ ID NO. 1070 | SEQ ID NO. 1071 | SEQ ID NO. 1072 |
| 1.269 | SEQ ID NO. 1073 | SEQ ID NO. 1074 | SEQ ID NO. 1075 | SEQ ID NO. 1076 |
| 1.270 | SEQ ID NO. 1077 | SEQ ID NO. 1078 | SEQ ID NO. 1079 | SEQ ID NO. 1080 |
| 1.271 | SEQ ID NO. 1081 | SEQ ID NO. 1082 | SEQ ID NO. 1083 | SEQ ID NO. 1084 |
| 1.272 | SEQ ID NO. 1085 | SEQ ID NO. 1086 | SEQ ID NO. 1087 | SEQ ID NO. 1088 |
| 1.273 | SEQ ID NO. 1089 | SEQ ID NO. 1090 | SEQ ID NO. 1091 | SEQ ID NO. 1092 |
| 1.274 | SEQ ID NO. 1093 | SEQ ID NO. 1094 | SEQ ID NO. 1095 | SEQ ID NO. 1096 |
| 1.275 | SEQ ID NO. 1097 | SEQ ID NO. 1098 | SEQ ID NO. 1099 | SEQ ID NO. 1100 |
| 1.276 | SEQ ID NO. 1101 | SEQ ID NO. 1102 | SEQ ID NO. 1103 | SEQ ID NO. 1104 |
| 1.277 | SEQ ID NO. 1105 | SEQ ID NO. 1106 | SEQ ID NO. 1107 | SEQ ID NO. 1108 |
| 1.278 | SEQ ID NO. 1109 | SEQ ID NO. 1110 | SEQ ID NO. 1111 | SEQ ID NO. 1112 |
| 1.279 | SEQ ID NO. 1113 | SEQ ID NO. 1114 | SEQ ID NO. 1115 | SEQ ID NO. 1116 |
| 1.280 | SEQ ID NO. 1117 | SEQ ID NO. 1118 | SEQ ID NO. 1119 | SEQ ID NO. 1120 |
| 1.281 | SEQ ID NO. 1121 | SEQ ID NO. 1122 | SEQ ID NO. 1123 | SEQ ID NO. 1124 |
| 1.282 | SEQ ID NO. 1125 | SEQ ID NO. 1126 | SEQ ID NO. 1127 | SEQ ID NO. 1128 |
| 1.283 | SEQ ID NO. 1129 | SEQ ID NO. 1130 | SEQ ID NO. 1131 | SEQ ID NO. 1132 |
| 1.284 | SEQ ID NO. 1133 | SEQ ID NO. 1134 | SEQ ID NO. 1135 | SEQ ID NO. 1136 |
| 1.285 | SEQ ID NO. 1137 | SEQ ID NO. 1138 | SEQ ID NO. 1139 | SEQ ID NO. 1140 |
| 1.286 | SEQ ID NO. 1141 | SEQ ID NO. 1142 | SEQ ID NO. 1143 | SEQ ID NO. 1144 |
| 1.287 | SEQ ID NO. 1145 | SEQ ID NO. 1146 | SEQ ID NO. 1147 | SEQ ID NO. 1148 |
| 1.288 | SEQ ID NO. 1149 | SEQ ID NO. 1150 | SEQ ID NO. 1151 | SEQ ID NO. 1152 |
| 1.289 | SEQ ID NO. 1153 | SEQ ID NO. 1154 | SEQ ID NO. 1155 | SEQ ID NO. 1156 |
| 1.290 | SEQ ID NO. 1157 | SEQ ID NO. 1158 | SEQ ID NO. 1159 | SEQ ID NO. 1160 |
| 1.291 | SEQ ID NO. 1161 | SEQ ID NO. 1162 | SEQ ID NO. 1163 | SEQ ID NO. 1164 |
| 1.292 | SEQ ID NO. 1165 | SEQ ID NO. 1166 | SEQ ID NO. 1167 | SEQ ID NO. 1168 |
| 1.293 | SEQ ID NO. 1169 | SEQ ID NO. 1170 | SEQ ID NO. 1171 | SEQ ID NO. 1172 |
| 1.294 | SEQ ID NO. 1173 | SEQ ID NO. 1174 | SEQ ID NO. 1175 | SEQ ID NO. 1176 |
| 1.295 | SEQ ID NO. 1177 | SEQ ID NO. 1178 | SEQ ID NO. 1179 | SEQ ID NO. 1180 |
| 1.296 | SEQ ID NO. 1181 | SEQ ID NO. 1182 | SEQ ID NO. 1183 | SEQ ID NO. 1184 |
| 1.297 | SEQ ID NO. 1185 | SEQ ID NO. 1186 | SEQ ID NO. 1187 | SEQ ID NO. 1188 |
| 1.298 | SEQ ID NO. 1189 | SEQ ID NO. 1190 | SEQ ID NO. 1191 | SEQ ID NO. 1192 |
| 1.299 | SEQ ID NO. 1193 | SEQ ID NO. 1194 | SEQ ID NO. 1195 | SEQ ID NO. 1196 |
| 1.300 | SEQ ID NO. 1197 | SEQ ID NO. 1198 | SEQ ID NO. 1199 | SEQ ID NO. 1200 |
| 1.301 | SEQ ID NO. 1201 | SEQ ID NO. 1202 | SEQ ID NO. 1203 | SEQ ID NO. 1204 |
| 1.302 | SEQ ID NO. 1205 | SEQ ID NO. 1206 | SEQ ID NO. 1207 | SEQ ID NO. 1208 |
| 1.303 | SEQ ID NO. 1209 | SEQ ID NO. 1210 | SEQ ID NO. 1211 | SEQ ID NO. 1212 |
| 1.304 | SEQ ID NO. 1213 | SEQ ID NO. 1214 | SEQ ID NO. 1215 | SEQ ID NO. 1216 |
| 1.305 | SEQ ID NO. 1217 | SEQ ID NO. 1218 | SEQ ID NO. 1219 | SEQ ID NO. 1220 |
| 1.306 | SEQ ID NO. 1221 | SEQ ID NO. 1222 | SEQ ID NO. 1223 | SEQ ID NO. 1224 |
| 1.307 | SEQ ID NO. 1225 | SEQ ID NO. 1226 | SEQ ID NO. 1227 | SEQ ID NO. 1228 |
| 1.308 | SEQ ID NO. 1229 | SEQ ID NO. 1230 | SEQ ID NO. 1231 | SEQ ID NO. 1232 |
| 1.309 | SEQ ID NO. 1233 | SEQ ID NO. 1234 | SEQ ID NO. 1235 | SEQ ID NO. 1236 |
| 1.310 | SEQ ID NO. 1237 | SEQ ID NO. 1238 | SEQ ID NO. 1239 | SEQ ID NO. 1240 |
| 1.311 | SEQ ID NO. 1241 | SEQ ID NO. 1242 | SEQ ID NO. 1243 | SEQ ID NO. 1244 |
| 1.312 | SEQ ID NO. 1245 | SEQ ID NO. 1246 | SEQ ID NO. 1247 | SEQ ID NO. 1248 |
| 1.313 | SEQ ID NO. 1249 | SEQ ID NO. 1250 | SEQ ID NO. 1251 | SEQ ID NO. 1252 |
| 1.314 | SEQ ID NO. 1253 | SEQ ID NO. 1254 | SEQ ID NO. 1255 | SEQ ID NO. 1256 |
| 1.315 | SEQ ID NO. 1257 | SEQ ID NO. 1258 | SEQ ID NO. 1259 | SEQ ID NO. 1260 |
| 1.316 | SEQ ID NO. 1261 | SEQ ID NO. 1262 | SEQ ID NO. 1263 | SEQ ID NO. 1264 |

In one aspect of the invention, the family 1 or family 1-like binding molecule comprises a human V_{H} domain comprising a CDR3 sequence comprising SEQ ID NO. 3 or a sequence with at least 70%, at least 80%, at least 90%, or at least 95% homology to SEQ ID NO. 3.

In one embodiment, the family 1 or family 1-like binding molecule comprises at least one immunoglobulin single domain antibody directed against IL-17RA wherein said domain is a human V_{H} domain and wherein said V_{H} domain comprises at least one antigen binding site comprising a CDR3 sequence having SEQ ID NO. 3 or a sequence with at least 70%, at least 80%, at least 90%, or at least 95% homology to SEQ ID NO. 3.

In one embodiment, homology is at least 90% homology to SEQ ID NO. 3.

In one embodiment, the CDR3 sequence is selected from one of the CDR3 sequences as shown in table 1 with reference to FIG. 1. Thus, the CDR3 sequence is selected from SEQ ID Nos. 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 30, 307, 311, 315, 319, 323, 327, 331, 335, 339, 343, 347, 351, 355, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503. 507, 511, 515, 519, 523, 527, 531, 535, 539, 543, 547, 551, 555, 563, 567, 571, 575, 579, 583, 587, 591, 595, 599, 603, 607, 611, 615, 619, 623, 627, 631, 635, 639, 643, 647, 651, 655, 659, 663, 667, 675, 679, 683, 687, 691, 695, 699, 703, 707, 711, 715, 723, 727, 731, 735, 739, 743, 747, 751, 755, 759, 763, 767, 771, 775, 779, 783, 787, 791, 795, 799, 803, 807, 811, 815, 819, 823, 827, 831, 835, 839, 843, 847, 851, 855, 859, 871, 875, 879, 883, 887, 891, 895, 899, 903, 907, 911, 915, 919, 923, 927, 931, 935, 939, 943, 947, 951, 955, 959, 963, 967, 971, 975, 979, 983, 987, 991, 995, 999, 1003, 1007, 1011, 1015, 1019, 1023, 1027, 1031, 1035, 1039, 1043, 1047, 1051, 1055, 1059, 1063, 1067, 1071, 1075, 1079, 1083, 1087, 1091, 1095, 1099, 1103, 1107, 1111, 1115, 1119, 1123, 1127, 1131, 1135, 1139, 1143, 1147, 1151, 1155, 1159, 1163, 1167, 1171, 1175, 1179, 1183, 1187, 1191, 1195, 1199, 1203, 1207, 1211, 1215, 1219, 1223, 1227, 1231, 1235, 1239, 1243, 1247, 1251, 1259 or 1263.

In one embodiment, the family 1 or family 1-like binding molecule comprises at least one antigen binding site comprising CDR1, CDR2 and CDR3, said CDR1 region comprising or consisting of amino acid sequence SEQ ID NO. 1 or a sequence with at least 70%, at least 80%, at least 90%, or at least 95% homology thereto, said CDR2 region comprising or consisting of the amino acid sequence SEQ ID NO. 2 or a sequence with at least 70%, at least 80%, at least 90%, or at least 95% homology thereto, and said CDR3 region comprising or consisting of the amino acid sequence SEQ ID NO. 3 or a sequence with at least 60%, at least 70%, at least 80%, at least 90%, or at least 95% homology thereto. For example, the CDR sequence may be a CDR sequence selected from those shown in FIG. 1.

In one embodiment, said CDR1 comprises or consists of the amino acid sequence as shown in SEQ ID NO. 1 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR2 comprises or consists of the amino acid sequence as shown in SEQ ID NO: 2 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR3 comprises or consists of the amino acid sequence as shown in SEQ ID NO. 3 or a sequence with at least 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99% homology thereto.

In one embodiment, CDR1 comprises or consists of one of the CDR1 amino acid sequence listed above in table 1 with reference to FIG. 1, CDR2 comprises or consists of one of the CDR2 amino acid sequence listed above in table 1 with reference to FIG. 1 and CDR3 comprises or consists of one of the CDR3 amino acid sequence listed above in table 1 with reference to FIG. 1. In one embodiment, the binding molecule has combinations of CDR1, CDR2 and CDR3 as shown for clones 1.1 to 1.316 in FIG. 1. In one embodiment, the binding molecule has combinations of CDR1, CDR2 and CDR3 as shown for clones 1.1 to 1.6 in FIG. 1. In one embodiment, the binding molecule comprises a set of CDR1, CDR2 and CDR3 sequences of a V_{H} sequence as shown for clones 1.1 to 1.316 in FIG. 1. In one embodiment, the binding molecule has a set of CDR1, CDR2 and CDR3 sequences of a V_{H} sequence as shown for clones 1.1 to 1.6 in FIG. 1.

Thus, in one embodiment, CDR1 is SEQ ID NO. 1, CDR2 is SEQ ID NO. 2 and CDR3 is SEQ ID NO. 3. In another embodiment, CDR1 is SEQ ID NO. 5, CDR2 is SEQ ID NO. 6 and CDR3 is SEQ ID NO. 7. In another embodiment, CDR1 is SEQ ID NO. 9, CDR2 is SEQ ID NO. 10 and CDR3 is SEQ ID NO. 11. In another embodiment, CDR1 is SEQ ID NO. 13, CDR2 is SEQ ID NO. 14 and CDR3 is SEQ ID NO. 15. In another embodiment, CDR1 is SEQ ID NO. 17, CDR2 is SEQ ID NO. 18 and CDR3 is SEQ ID NO. 19. In another embodiment, CDR1 is SEQ ID NO. 21, CDR2 is SEQ ID NO. 22 and CDR3 is SEQ ID NO. 23.

In one embodiment, the family 1 or family 1-like sequence has a V_{H} domain that comprises or consists of SEQ ID NO. 4 or a sequence with at least 40%, 50%, 60%, 70%, 80%, 81%, 82%, 83%, 84%, 85%, 86%, 87%, 88%, 89%, 90%, 91%, 92%, 93%, 94%, 95% 96%, 97%, 98% or 99% homology thereto. CDR sequences of such sequences are shown in FIG. 1. For example, the V_{H} domain comprises or consists of one of the V_{H} amino acid sequences listed above for clones 1.1 to 1.316 in table 1 with reference to FIG. 1. Thus, the V_{H} sequence comprises or consists of a sequence selected from SEQ ID NOs. 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784. 788, 792, 796, 800, 804, 808, 812, 816, 820, 824, 828, 832, 836, 840, 844, 848, 852, 856, 860, 864, 868, 872, 876, 880, 884, 888, 892 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260 or 1264. In another embodiment, the V_{H} sequence is selected from one of the sequences in the forgoing but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences. In one embodiment, the V_{H} domain comprises or consists of SEQ ID NO. 4 or a sequence which comprises one or more amino acid substitutions, for example 1, 2, 3, 4, 5, 6, 7, 8, 9 or 10 amino acid substitutions.

In one embodiment, said V_{H} domain comprises or consists of a sequence selected from SEQ ID NOs. 4, 8, 12, 16, 20 or 24 or a sequence with at least 40%, 50%, 60%, 70%, 80%, 81%, 82%, 83%, 84%, 85%, 86%, 87%, 88%, 89%, 90%, 91%, 92%, 93%, 94%, 95% 96%, 97%, 98% or 99% homology thereto.

In another aspect, the invention relates to a binding molecule comprising or consisting of a V_{H} domain as shown in SEQ ID NO. 4 or a variant thereof comprising amino acid substitutions compared to SEQ ID NO. 4 as follows residue 1 is E, residue 30 is A, residue 55 is A, residue 58 is K, I, residue 59 is G, residue 63 is T, residue 100 is S, residue 101 is S, residue 104 is Y and/or residue 106 is S.

The family 1 or family 1-like binding molecules preferably have KD, Koff, KA, Kd and IC₅₀ values as further described herein and as shown in the examples.

The term “KD” refers to the “equilibrium dissociation constant” and refers to the value obtained in a titration measurement at equilibrium, or by dividing the dissociation rate constant (Koff) by the association rate constant (Kon). “KA” refers to the affinity constant. The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used.

In another aspect, the invention relates to a binding molecule capable of binding human IL-17RA comprising a human V_{H} domain wherein said V_{H} domain comprises a family 2 or family 2-like sequence.

In one embodiment, the binding molecule comprises or consists of at least one immunoglobulin single domain antibody directed against IL-17RA, preferably human IL-17RA, wherein said domain is a human V_{H} domain and wherein said IL-17RA binding molecule comprises a family 2 or family 2-like sequence. These include the parent sequence and sequences of clones that are derived from the parent clone (clone 2.2) or a part thereof, for example a CDR3 sequence, and V_{H} sequences of clones or parts thereof that are derived from the parent clone 2.1 through a process of optimization, for example as shown in FIG. 2. CDR sequences and full length sequences of clones in family 2 are numbered according to table 2 as shown below.

TABLE 2
This shows SEQ ID NOs of family 2 CDR sequences and of family
2 full-length V_{H} sequences that are within the scope of the
invention. Corresponding sequences are shown in FIG. 2.

| Clone | | | | VH Full length |
|---|---|---|---|---|
| name | CDR1 Seq ID No | CDR2 Seq ID No | CDR3 Seq ID No | sequence Seq ID No |
| 2.1 | SEQ ID NO. 1265 | SEQ ID NO. 1266 | SEQ ID NO. 1267 | SEQ ID NO. 1268 |
| 2.2 | SEQ ID NO. 1269 | SEQ ID NO. 1270 | SEQ ID NO. 1271 | SEQ ID NO. 1272 |
| 2.3 | SEQ ID NO. 1273 | SEQ ID NO. 1274 | SEQ ID NO. 1275 | SEQ ID NO. 1276 |
| 2.4 | SEQ ID NO. 1277 | SEQ ID NO. 1278 | SEQ ID NO. 1279 | SEQ ID NO. 1280 |
| 2.5 | SEQ ID NO. 1281 | SEQ ID NO. 1282 | SEQ ID NO. 1283 | SEQ ID NO. 1284 |
| 2.6 | SEQ ID NO. 1285 | SEQ ID NO. 1286 | SEQ ID NO. 1287 | SEQ ID NO. 1288 |
| 2.7 | SEQ ID NO. 1289 | SEQ ID NO. 1290 | SEQ ID NO. 1291 | SEQ ID NO. 1292 |
| 2.8 | SEQ ID NO. 1293 | SEQ ID NO. 1294 | SEQ ID NO. 1295 | SEQ ID NO. 1296 |
| 2.9 | SEQ ID NO. 1297 | SEQ ID NO. 1298 | SEQ ID NO. 1299 | SEQ ID NO. 1300 |
| 2.10 | SEQ ID NO. 1301 | SEQ ID NO. 1302 | SEQ ID NO. 1303 | SEQ ID NO. 1304 |
| 2.11 | SEQ ID NO. 1305 | SEQ ID NO. 1306 | SEQ ID NO. 1307 | SEQ ID NO. 1308 |
| 2.12 | SEQ ID NO. 1309 | SEQ ID NO. 1310 | SEQ ID NO. 1311 | SEQ ID NO. 1312 |
| 2.13 | SEQ ID NO. 1313 | SEQ ID NO. 1314 | SEQ ID NO. 1315 | SEQ ID NO. 1316 |
| 2.14 | SEQ ID NO. 1317 | SEQ ID NO. 1318 | SEQ ID NO. 1319 | SEQ ID NO. 1320 |
| 2.15 | SEQ ID NO. 1321 | SEQ ID NO. 1322 | SEQ ID NO. 1323 | SEQ ID NO. 1324 |
| 2.16 | SEQ ID NO. 1325 | SEQ ID NO. 1326 | SEQ ID NO. 1327 | SEQ ID NO. 1328 |
| 2.17 | SEQ ID NO. 1329 | SEQ ID NO. 1330 | SEQ ID NO. 1331 | SEQ ID NO. 1332 |
| 2.18 | SEQ ID NO. 1333 | SEQ ID NO. 1334 | SEQ ID NO. 1335 | SEQ ID NO. 1336 |
| 2.19 | SEQ ID NO. 1337 | SEQ ID NO. 1338 | SEQ ID NO. 1339 | SEQ ID NO. 1340 |
| 2.20 | SEQ ID NO. 1341 | SEQ ID NO. 1342 | SEQ ID NO. 1343 | SEQ ID NO. 1344 |
| 2.21 | SEQ ID NO. 1345 | SEQ ID NO. 1346 | SEQ ID NO. 1347 | SEQ ID NO. 1348 |
| 2.22 | SEQ ID NO. 1349 | SEQ ID NO. 1350 | SEQ ID NO. 1351 | SEQ ID NO. 1352 |
| 2.23 | SEQ ID NO. 1353 | SEQ ID NO. 1354 | SEQ ID NO. 1355 | SEQ ID NO. 1356 |
| 2.24 | SEQ ID NO. 1357 | SEQ ID NO. 1358 | SEQ ID NO. 1359 | SEQ ID NO. 1360 |
| 2.25 | SEQ ID NO. 1361 | SEQ ID NO. 1362 | SEQ ID NO. 1363 | SEQ ID NO. 1364 |
| 2.26 | SEQ ID NO. 1365 | SEQ ID NO. 1366 | SEQ ID NO. 1367 | SEQ ID NO. 1368 |
| 2.27 | SEQ ID NO. 1369 | SEQ ID NO. 1370 | SEQ ID NO. 1371 | SEQ ID NO. 1372 |
| 2.28 | SEQ ID NO. 1373 | SEQ ID NO. 1374 | SEQ ID NO. 1375 | SEQ ID NO. 1376 |
| 2.29 | SEQ ID NO. 1377 | SEQ ID NO. 1378 | SEQ ID NO. 1379 | SEQ ID NO. 1380 |
| 2.30 | SEQ ID NO. 1381 | SEQ ID NO. 1382 | SEQ ID NO. 1383 | SEQ ID NO. 1384 |
| 2.31 | SEQ ID NO. 1385 | SEQ ID NO. 1386 | SEQ ID NO. 1387 | SEQ ID NO. 1388 |
| 2.32 | SEQ ID NO. 1389 | SEQ ID NO. 1390 | SEQ ID NO. 1391 | SEQ ID NO. 1392 |
| 2.33 | SEQ ID NO. 1393 | SEQ ID NO. 1394 | SEQ ID NO. 1395 | SEQ ID NO. 1396 |
| 2.34 | SEQ ID NO. 1397 | SEQ ID NO. 1398 | SEQ ID NO. 1399 | SEQ ID NO. 1400 |
| 2.35 | SEQ ID NO. 1401 | SEQ ID NO. 1402 | SEQ ID NO. 1403 | SEQ ID NO. 1404 |
| 2.36 | SEQ ID NO. 1405 | SEQ ID NO. 1406 | SEQ ID NO. 1407 | SEQ ID NO. 1408 |
| 2.37 | SEQ ID NO. 1409 | SEQ ID NO. 1410 | SEQ ID NO. 1411 | SEQ ID NO. 1412 |
| 2.38 | SEQ ID NO. 1413 | SEQ ID NO. 1414 | SEQ ID NO. 1415 | SEQ ID NO. 1416 |
| 2.39 | SEQ ID NO. 1417 | SEQ ID NO. 1418 | SEQ ID NO. 1419 | SEQ ID NO. 1420 |
| 2.40 | SEQ ID NO. 1421 | SEQ ID NO. 1422 | SEQ ID NO. 1423 | SEQ ID NO. 1424 |
| 2.41 | SEQ ID NO. 1425 | SEQ ID NO. 1426 | SEQ ID NO. 1427 | SEQ ID NO. 1428 |
| 2.42 | SEQ ID NO. 1429 | SEQ ID NO. 1430 | SEQ ID NO. 1431 | SEQ ID NO. 1432 |
| 2.43 | SEQ ID NO. 1433 | SEQ ID NO. 1434 | SEQ ID NO. 1435 | SEQ ID NO. 1436 |
| 2.44 | SEQ ID NO. 1437 | SEQ ID NO. 1438 | SEQ ID NO. 1439 | SEQ ID NO. 1440 |
| 2.45 | SEQ ID NO. 1441 | SEQ ID NO. 1442 | SEQ ID NO. 1443 | SEQ ID NO. 1444 |
| 2.46 | SEQ ID NO. 1445 | SEQ ID NO. 1446 | SEQ ID NO. 1447 | SEQ ID NO. 1448 |
| 2.47 | SEQ ID NO. 1449 | SEQ ID NO. 1450 | SEQ ID NO. 1451 | SEQ ID NO. 1452 |
| 2.48 | SEQ ID NO. 1453 | SEQ ID NO. 1454 | SEQ ID NO. 1455 | SEQ ID NO. 1456 |
| 2.49 | SEQ ID NO. 1457 | SEQ ID NO. 1458 | SEQ ID NO. 1459 | SEQ ID NO. 1460 |
| 2.50 | SEQ ID NO. 1461 | SEQ ID NO. 1462 | SEQ ID NO. 1463 | SEQ ID NO. 1464 |
| 2.51 | SEQ ID NO. 1465 | SEQ ID NO. 1466 | SEQ ID NO. 1467 | SEQ ID NO. 1468 |
| 2.52 | SEQ ID NO. 1469 | SEQ ID NO. 1470 | SEQ ID NO. 1471 | SEQ ID NO. 1472 |
| 2.53 | SEQ ID NO. 1473 | SEQ ID NO. 1474 | SEQ ID NO. 1475 | SEQ ID NO. 1476 |
| 2.54 | SEQ ID NO. 1477 | SEQ ID NO. 1478 | SEQ ID NO. 1479 | SEQ ID NO. 1480 |
| 2.55 | SEQ ID NO. 1481 | SEQ ID NO. 1482 | SEQ ID NO. 1483 | SEQ ID NO. 1484 |
| 2.56 | SEQ ID NO. 1485 | SEQ ID NO. 1486 | SEQ ID NO. 1487 | SEQ ID NO. 1488 |
| 2.57 | SEQ ID NO. 1489 | SEQ ID NO. 1490 | SEQ ID NO. 1491 | SEQ ID NO. 1492 |
| 2.58 | SEQ ID NO. 1493 | SEQ ID NO. 1494 | SEQ ID NO. 1495 | SEQ ID NO. 1496 |
| 2.59 | SEQ ID NO. 1497 | SEQ ID NO. 1498 | SEQ ID NO. 1499 | SEQ ID NO. 1500 |
| 2.60 | SEQ ID NO. 1501 | SEQ ID NO. 1502 | SEQ ID NO. 1503 | SEQ ID NO. 1504 |
| 2.61 | SEQ ID NO. 1505 | SEQ ID NO. 1506 | SEQ ID NO. 1507 | SEQ ID NO. 1508 |
| 2.62 | SEQ ID NO. 1509 | SEQ ID NO. 1510 | SEQ ID NO. 1511 | SEQ ID NO. 1512 |
| 2.63 | SEQ ID NO. 1513 | SEQ ID NO. 1514 | SEQ ID NO. 1515 | SEQ ID NO. 1516 |
| 2.64 | SEQ ID NO. 1517 | SEQ ID NO. 1518 | SEQ ID NO. 1519 | SEQ ID NO. 1520 |
| 2.65 | SEQ ID NO. 1521 | SEQ ID NO. 1522 | SEQ ID NO. 1523 | SEQ ID NO. 1524 |
| 2.66 | SEQ ID NO. 1525 | SEQ ID NO. 1526 | SEQ ID NO. 1527 | SEQ ID NO. 1528 |
| 2.67 | SEQ ID NO. 1529 | SEQ ID NO. 1530 | SEQ ID NO. 1531 | SEQ ID NO. 1532 |
| 2.68 | SEQ ID NO. 1533 | SEQ ID NO. 1534 | SEQ ID NO. 1535 | SEQ ID NO. 1536 |
| 2.69 | SEQ ID NO. 1537 | SEQ ID NO. 1538 | SEQ ID NO. 1539 | SEQ ID NO. 1540 |
| 2.70 | SEQ ID NO. 1541 | SEQ ID NO. 1542 | SEQ ID NO. 1543 | SEQ ID NO. 1544 |
| 2.71 | SEQ ID NO. 1545 | SEQ ID NO. 1546 | SEQ ID NO. 1547 | SEQ ID NO. 1548 |
| 2.72 | SEQ ID NO. 1549 | SEQ ID NO. 1550 | SEQ ID NO. 1551 | SEQ ID NO. 1552 |
| 2.73 | SEQ ID NO. 1553 | SEQ ID NO. 1554 | SEQ ID NO. 1555 | SEQ ID NO. 1556 |
| 2.74 | SEQ ID NO. 1557 | SEQ ID NO. 1558 | SEQ ID NO. 1559 | SEQ ID NO. 1560 |
| 2.75 | SEQ ID NO. 1561 | SEQ ID NO. 1562 | SEQ ID NO. 1563 | SEQ ID NO. 1564 |
| 2.76 | SEQ ID NO. 1565 | SEQ ID NO. 1566 | SEQ ID NO. 1567 | SEQ ID NO. 1568 |
| 2.77 | SEQ ID NO. 1569 | SEQ ID NO. 1570 | SEQ ID NO. 1571 | SEQ ID NO. 1572 |
| 2.78 | SEQ ID NO. 1573 | SEQ ID NO. 1574 | SEQ ID NO. 1575 | SEQ ID NO. 1576 |
| 2.79 | SEQ ID NO. 1577 | SEQ ID NO. 1578 | SEQ ID NO. 1579 | SEQ ID NO. 1580 |
| 2.80 | SEQ ID NO. 1581 | SEQ ID NO. 1582 | SEQ ID NO. 1583 | SEQ ID NO. 1584 |
| 2.81 | SEQ ID NO. 1585 | SEQ ID NO. 1586 | SEQ ID NO. 1587 | SEQ ID NO. 1588 |
| 2.82 | SEQ ID NO. 1589 | SEQ ID NO. 1590 | SEQ ID NO. 1591 | SEQ ID NO. 1592 |
| 2.83 | SEQ ID NO. 1593 | SEQ ID NO. 1594 | SEQ ID NO. 1595 | SEQ ID NO. 1596 |
| 2.84 | SEQ ID NO. 1597 | SEQ ID NO. 1598 | SEQ ID NO. 1599 | SEQ ID NO. 1600 |
| 2.85 | SEQ ID NO. 1601 | SEQ ID NO. 1602 | SEQ ID NO. 1603 | SEQ ID NO. 1604 |
| 2.86 | SEQ ID NO. 1605 | SEQ ID NO. 1606 | SEQ ID NO. 1607 | SEQ ID NO. 1608 |
| 2.87 | SEQ ID NO. 1609 | SEQ ID NO. 1610 | SEQ ID NO. 1611 | SEQ ID NO. 1612 |
| 2.88 | SEQ ID NO. 1613 | SEQ ID NO. 1614 | SEQ ID NO. 1615 | SEQ ID NO. 1616 |
| 2.89 | SEQ ID NO. 1617 | SEQ ID NO. 1618 | SEQ ID NO. 1619 | SEQ ID NO. 1620 |
| 2.90 | SEQ ID NO. 1621 | SEQ ID NO. 1622 | SEQ ID NO. 1623 | SEQ ID NO. 1624 |
| 2.91 | SEQ ID NO. 1625 | SEQ ID NO. 1626 | SEQ ID NO. 1627 | SEQ ID NO. 1628 |
| 2.92 | SEQ ID NO. 1629 | SEQ ID NO. 1630 | SEQ ID NO. 1631 | SEQ ID NO. 1632 |
| 2.93 | SEQ ID NO. 1633 | SEQ ID NO. 1634 | SEQ ID NO. 1635 | SEQ ID NO. 1636 |
| 2.94 | SEQ ID NO. 1637 | SEQ ID NO. 1638 | SEQ ID NO. 1639 | SEQ ID NO. 1640 |
| 2.95 | SEQ ID NO. 1641 | SEQ ID NO. 1642 | SEQ ID NO. 1643 | SEQ ID NO. 1644 |
| 2.96 | SEQ ID NO. 1645 | SEQ ID NO. 1646 | SEQ ID NO. 1647 | SEQ ID NO. 1648 |
| 2.97 | SEQ ID NO. 1649 | SEQ ID NO. 1650 | SEQ ID NO. 1651 | SEQ ID NO. 1652 |
| 2.98 | SEQ ID NO. 1653 | SEQ ID NO. 1654 | SEQ ID NO. 1655 | SEQ ID NO. 1656 |
| 2.99 | SEQ ID NO. 1657 | SEQ ID NO. 1658 | SEQ ID NO. 1659 | SEQ ID NO. 1660 |
| 2.100 | SEQ ID NO. 1661 | SEQ ID NO. 1662 | SEQ ID NO. 1663 | SEQ ID NO. 1664 |
| 2.101 | SEQ ID NO. 1665 | SEQ ID NO. 1666 | SEQ ID NO. 1667 | SEQ ID NO. 1668 |
| 2.102 | SEQ ID NO. 1669 | SEQ ID NO. 1670 | SEQ ID NO. 1671 | SEQ ID NO. 1672 |
| 2.103 | SEQ ID NO. 1673 | SEQ ID NO. 1674 | SEQ ID NO. 1675 | SEQ ID NO. 1676 |
| 2.104 | SEQ ID NO. 1677 | SEQ ID NO. 1678 | SEQ ID NO. 1679 | SEQ ID NO. 1680 |
| 2.105 | SEQ ID NO. 1681 | SEQ ID NO. 1682 | SEQ ID NO. 1683 | SEQ ID NO. 1684 |
| 2.106 | SEQ ID NO. 1685 | SEQ ID NO. 1686 | SEQ ID NO. 1687 | SEQ ID NO. 1688 |
| 2.107 | SEQ ID NO. 1689 | SEQ ID NO. 1690 | SEQ ID NO. 1691 | SEQ ID NO. 1692 |
| 2.108 | SEQ ID NO. 1693 | SEQ ID NO. 1694 | SEQ ID NO. 1695 | SEQ ID NO. 1696 |
| 2.109 | SEQ ID NO. 1697 | SEQ ID NO. 1698 | SEQ ID NO. 1699 | SEQ ID NO. 1700 |
| 2.110 | SEQ ID NO. 1701 | SEQ ID NO. 1702 | SEQ ID NO. 1703 | SEQ ID NO. 1704 |
| 2.111 | SEQ ID NO. 1705 | SEQ ID NO. 1706 | SEQ ID NO. 1707 | SEQ ID NO. 1708 |
| 2.112 | SEQ ID NO. 1709 | SEQ ID NO. 1710 | SEQ ID NO. 1711 | SEQ ID NO. 1712 |
| 2.113 | SEQ ID NO. 1713 | SEQ ID NO. 1714 | SEQ ID NO. 1715 | SEQ ID NO. 1716 |
| 2.114 | SEQ ID NO. 1717 | SEQ ID NO. 1718 | SEQ ID NO. 1719 | SEQ ID NO. 1720 |
| 2.115 | SEQ ID NO. 1721 | SEQ ID NO. 1722 | SEQ ID NO. 1723 | SEQ ID NO. 1724 |
| 2.116 | SEQ ID NO. 1725 | SEQ ID NO. 1726 | SEQ ID NO. 1727 | SEQ ID NO. 1728 |
| 2.117 | SEQ ID NO. 1729 | SEQ ID NO. 1730 | SEQ ID NO. 1731 | SEQ ID NO. 1732 |
| 2.118 | SEQ ID NO. 1733 | SEQ ID NO. 1734 | SEQ ID NO. 1735 | SEQ ID NO. 1736 |
| 2.119 | SEQ ID NO. 1737 | SEQ ID NO. 1738 | SEQ ID NO. 1739 | SEQ ID NO. 1740 |
| 2.120 | SEQ ID NO. 1741 | SEQ ID NO. 1742 | SEQ ID NO. 1743 | SEQ ID NO. 1744 |
| 2.121 | SEQ ID NO. 1745 | SEQ ID NO. 1746 | SEQ ID NO. 1747 | SEQ ID NO. 1748 |
| 2.122 | SEQ ID NO. 1749 | SEQ ID NO. 1750 | SEQ ID NO. 1751 | SEQ ID NO. 1752 |
| 2.123 | SEQ ID NO. 1753 | SEQ ID NO. 1754 | SEQ ID NO. 1755 | SEQ ID NO. 1756 |
| 2.124 | SEQ ID NO. 1757 | SEQ ID NO. 1758 | SEQ ID NO. 1759 | SEQ ID NO. 1760 |
| 2.125 | SEQ ID NO. 1761 | SEQ ID NO. 1762 | SEQ ID NO. 1763 | SEQ ID NO. 1764 |

In one aspect, the invention relates to a family 2 or family 2-like binding molecule comprises a human V_{H} domain comprising a CDR3 sequence comprising SEQ ID NO. 1237 or a sequence with at least 70%, at least 80%, at least 90%, or at least 95% homology to SEQ ID NO. 1237.

In one embodiment, the family 2 or family 2-like binding molecule comprises at least one immunoglobulin single domain antibody directed against IL-17RA wherein said domain is a human V_{H} domain and wherein said V_{H} comprises at least one antigen binding site comprising a CDR3 sequence having SEQ ID NO. 1267 or a sequence with at least 70%, at least 80%, at least 90%, or at least 95% homology thereto. In one embodiment, sequence homology is at least 90%.

In one embodiment, the CDR3 sequence is selected from one of the CDR3 sequences as shown in table 2 with reference to FIG. 2. Thus, the CDR3 region comprises or consists of a sequence selected from SEQ ID Nos. 1267, 1271, 1275, 1279, 1283, 1287, 1291, 1295, 1299, 1303, 1307, 1311, 1315, 1319, 1323, 1327, 1331, 1335, 1339, 1343, 1347, 1351, 1355, 1359, 1363, 1367, 1371, 13751379, 1383, 1387, 1391, 1395, 1399, 1403, 1407, 1411, 1415, 1419, 1423, 1427, 1431, 1435, 1439, 1443, 1447, 1451, 1455, 1459, 1463, 1467, 1471, 1475, 1479, 1483, 1487, 1491, 1495, 1499, 1503, 1507, 1511, 1515, 1519, 1523, 1527, 1531, 1535, 1539, 1543, 1547, 1551, 1555, 1559, 1563, 1567, 1571, 1575, 1579, 1583, 1587, 1591, 1595, 1599, 1603, 1607, 1611, 1615, 1619, 1623, 1627, 1631, 1635, 1639, 1643, 1647, 1651, 1655, 1659, 1663, 1667, 1671, 1675, 1679, 1683, 1687, 1691, 1695, 1699, 1703, 1707, 1711, 1715, 1719, 1723, 1727, 1731, 1735, 1739, 1743, 1747, 1751, 1755, 1759 or 1763.

In one embodiment, the family 2 or family 2-like sequence comprises at least one antigen binding site comprising hypervariable regions CDR1, CDR2 and CDR3, said CDR1 comprises or consists of the amino acid sequence SEQ ID NO. 1265 or a sequence with at least 70%, at least 80%, at least 90%, or at least 95% homology thereto, said CDR2 comprises or consists of the amino acid sequence SEQ ID NO. 1266 or a sequence with at least 70%, at least 80%, at least 90%, or at least 95% homology thereto, and said CDR3 comprises or consists of the amino acid sequence SEQ ID NO. 1267 or a sequence with at least 70%, at least 80%, at least 90%, or at least 95% homology thereto. For example, the CDR sequence may be a CDR sequence selected from those shown in FIG. 2.

In one embodiment, said CDR1 comprises or consists of the amino acid sequence SEQ ID NO. 1265 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR2 comprises or consists of the amino acid sequence SEQ ID NO. 1266 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR3 comprises or consists of the amino acid sequence SEQ ID NO. 1267 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto.

In one embodiment, CDR1 comprises or consists of one of the CDR1 amino acid sequence listed above in table 2 with reference to FIG. 2, CDR2 comprises or consists of one of the CDR2 amino acid sequence listed above in table 2 with reference to FIG. 2 and CDR3 comprises or consists of one of the CDR3 amino acid sequence listed above in table 2 with reference to FIG. 2. In one embodiment, the binding molecule has combinations of CDR1, CDR2 and CDR3 as shown for clones 2.1 to 2.125 in FIG. 2. In one embodiment, the binding molecule has combinations of CDR1, CDR2 and CDR3 as shown for clones 2.1 to 2.3 in FIG. 2.

In one embodiment, the binding molecule comprises a set of CDR1, CDR2 and CDR3 sequences of a V_{H} sequence as shown for clones 2.1 to 2.125 in FIG. 2. In one embodiment, the binding molecule has a set of CDR1, CDR2 and CDR3 sequences of a V_{H} sequence as shown for clones 2.1 to 2.3 in FIG. 2.

Thus, in one embodiment, CDR1 is SEQ ID NO. 1269, CDR2 is SEQ ID NO. 1270 and CDR3 is SEQ ID NO. 1271. In another embodiment, CDR1 is SEQ ID NO. 1272, CDR2 is SEQ ID NO. 1273 and CDR3 is SEQ ID NO. 1274.

In one embodiment, the family 2 or family 2-like binding molecule has a V_{H} domain that comprises or consists of SEQ ID NO. 1268 or a sequence with at least 40%, 50%, 60%, 70%, 80%, 81%, 82%, 83%, 84%, 85%, 86%, 87%, 88%, 89%, 90%, 91%, 92%, 93%, 94%, 95% 96%, 97%, 98% or 99% homology thereto. CDR sequences of such sequences are shown in FIG. 2. For example, the V_{H} domain comprises or consists of one of the V_{H} amino acid sequences listed above for clones 2.1 to 2.125 in table 2 with reference to FIG. 2.

Thus, the V_{H} domain comprises or consists of a sequence selected from SEQ ID NOs. 1268, 1272, 1276, 1280, 1284, 1288, 1292, 1296, 1300, 1304, 1308, 1312, 1316, 1320 1324, 1328, 1332, 1336, 1340, 1344, 1348, 1352, 1356, 1360, 1364, 1368, 1372, 1376 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1476, 1480, 1484, 1488, 1492, 1496, 1500, 1504, 1508, 1512, 1516, 1520, 1524, 1528, 1532, 1536, 1540, 1544, 1548, 1552, 1556, 1560, 1564, 1568, 1572, 1576, 1580, 1584, 1588, 1592, 1596, 1600, 1604, 1608, 1612, 1616, 1620, 1624, 1628, 1632, 1636, 1640, 1644, 1648, 1652, 1656, 1660, 1664, 1668, 1672, 1676, 1680, 1684, 1688, 1692, 1696, 1700, 1704, 1708, 1712, 1716 1720, 1724, 1728, 1732, 1736, 1740, 1744, 1748, 1752, 1756, 1760 or 1764.

In another embodiment, the V_{H} domain comprises a sequence selected from one of the sequences in the forgoing but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences. In one embodiment, the V_{H} domain comprises or consists of SEQ ID NO. 1268 or a sequence which comprises one or more amino acid substitutions, for example 1, 2, 3, 4, 5, 6, 7, 8, 9 or 10 amino acid substitutions. For example, V_{H} domain comprises or consists of a sequence selected from SEQ ID NO. 1268 or 1272.

In one embodiment, the family 2 or family 2-like binding molecule has a V_{H} domain that comprises or consists of SEQ ID NO. 1268 or 1272, or a sequence with at least 40%, 50%, 60%, 70%, 80%, 81%, 82%, 83%, 84%, 85%, 87%, 88%, 89%, 90%, 91%, 92%, 93%, 94%, 96%, 97%, 98%, 99% homology thereto.

In another aspect, the invention relates to a binding molecule comprising or consisting of a V_{H} domain as shown in SEQ ID NO. 1268 or a variant thereof comprising amino acid substitutions compared to SEQ ID NO. 1268 as follows residue 31 is T, residue 43 is R, 54 is G, E, K, A, D, residue 55 is S, residue 57 is D, Y, N and/or residue 100 is I.

The family 2 or family 2-like binding molecules have KD, Koff, KA, Kd and IC₅₀ values as further described herein and as shown in the examples.

In one aspect, the invention relates to an isolated binding molecule capable of binding human IL-17RA comprising a human heavy chain variable immunoglobulin domain (V_{H}) wherein said V_{H} domain comprises a family 3 or family 3-like sequence.

In one embodiment, the binding molecule comprises or consists of at least one immunoglobulin single domain antibody directed against IL-17RA, preferably human IL-17RA, wherein said domain is a human V_{H} domain and wherein said VH domain comprises a family 3 or family 3-like sequence. These include the V_{H} sequence of the parent clone (clone 3.1) or a part thereof, for example a CDR3 sequence, and V_{H} sequences of clones or that are derived from the parent clone 3.1 through a process of optimization, for example as shown in FIG. 3. CDR sequences and full length sequences of clones in family 3 are numbered according to table 3 as shown below.

TABLE 3
This shows SEQ ID NOs of family 3-like CDR sequences and of
family full length V_{H} sequences that are within the scope of
the invention. Corresponding sequences are shown in FIG. 3.

| | | | | FULL LENGTH |
|---|---|---|---|---|
| Clone | CDR1 Seq ID No | CDR2 Seq ID No | CDR3 Seq ID No | Seq ID No |
| 3.1 | Seq ID No 1765 | Seq ID No 1766 | Seq ID No 1767 | Seq ID No 1768 |
| 3.2 | Seq ID No 1769 | Seq ID No 1770 | Seq ID No 1771 | Seq ID No 1772 |
| 3.3 | Seq ID No 1773 | Seq ID No 1774 | Seq ID No 1775 | Seq ID No 1776 |
| 3.4 | Seq ID No 1777 | Seq ID No 1778 | Seq ID No 1779 | Seq ID No 1780 |
| 3.5 | Seq ID No 1781 | Seq ID No 1782 | Seq ID No 1783 | Seq ID No 1784 |
| 3.6 | Seq ID No 1785 | Seq ID No 1786 | Seq ID No 1787 | Seq ID No 1788 |
| 3.7 | Seq ID No 1789 | Seq ID No 1790 | Seq ID No 1791 | Seq ID No 1792 |
| 3.8 | Seq ID No 1793 | Seq ID No 1794 | Seq ID No 1795 | Seq ID No 1796 |
| 3.9 | Seq ID No 1797 | Seq ID No 1798 | Seq ID No 1799 | Seq ID No 1800 |
| 3.10 | Seq ID No 1801 | Seq ID No 1802 | Seq ID No 1803 | Seq ID No 1804 |
| 3.11 | Seq ID No 1805 | Seq ID No 1806 | Seq ID No 1807 | Seq ID No 1808 |
| 3.12 | Seq ID No 1809 | Seq ID No 1810 | Seq ID No 1811 | Seq ID No 1812 |
| 3.13 | Seq ID No 1813 | Seq ID No 1814 | Seq ID No 1815 | Seq ID No 1816 |
| 3.14 | Seq ID No 1817 | Seq ID No 1818 | Seq ID No 1819 | Seq ID No 1820 |
| 3.15 | Seq ID No 1821 | Seq ID No 1822 | Seq ID No 1823 | Seq ID No 1824 |
| 3.16 | Seq ID No 1825 | Seq ID No 1826 | Seq ID No 1827 | Seq ID No 1828 |
| 3.17 | Seq ID No 1829 | Seq ID No 1830 | Seq ID No 1831 | Seq ID No 1832 |
| 3.18 | Seq ID No 1833 | Seq ID No 1834 | Seq ID No 1835 | Seq ID No 1836 |
| 3.19 | Seq ID No 1837 | Seq ID No 1838 | Seq ID No 1839 | Seq ID No 1840 |
| 3.20 | Seq ID No 1841 | Seq ID No 1842 | Seq ID No 1843 | Seq ID No 1844 |
| 3.21 | Seq ID No 1845 | Seq ID No 1846 | Seq ID No 1847 | Seq ID No 1848 |
| 3.22 | Seq ID No 1849 | Seq ID No 1850 | Seq ID No 1851 | Seq ID No 1852 |
| 3.23 | Seq ID No 1853 | Seq ID No 1854 | Seq ID No 1855 | Seq ID No 1856 |
| 3.24 | Seq ID No 1857 | Seq ID No 1858 | Seq ID No 1859 | Seq ID No 1860 |
| 3.25 | Seq ID No 1861 | Seq ID No 1862 | Seq ID No 1863 | Seq ID No 1864 |
| 3.26 | Seq ID No 1865 | Seq ID No 1866 | Seq ID No 1867 | Seq ID No 1868 |
| 3.27 | Seq ID No 1869 | Seq ID No 1870 | Seq ID No 1871 | Seq ID No 1872 |
| 3.28 | Seq ID No 1873 | Seq ID No 1874 | Seq ID No 1875 | Seq ID No 1876 |
| 3.29 | Seq ID No 1877 | Seq ID No 1878 | Seq ID No 1879 | Seq ID No 1880 |
| 3.30 | Seq ID No 1881 | Seq ID No 1882 | Seq ID No 1883 | Seq ID No 1884 |
| 3.31 | Seq ID No 1885 | Seq ID No 1886 | Seq ID No 1887 | Seq ID No 1888 |
| 3.32 | Seq ID No 1889 | Seq ID No 1890 | Seq ID No 1891 | Seq ID No 1892 |
| 3.33 | Seq ID No 1893 | Seq ID No 1894 | Seq ID No 1895 | Seq ID No 1896 |
| 3.34 | Seq ID No 1897 | Seq ID No 1898 | Seq ID No 1899 | Seq ID No 1900 |
| 3.35 | Seq ID No 1901 | Seq ID No 1902 | Seq ID No 1903 | Seq ID No 1904 |
| 3.36 | Seq ID No 1905 | Seq ID No 1906 | Seq ID No 1907 | Seq ID No 1908 |
| 3.37 | Seq ID No 1909 | Seq ID No 1910 | Seq ID No 1911 | Seq ID No 1912 |
| 3.38 | Seq ID No 1913 | Seq ID No 1914 | Seq ID No 1915 | Seq ID No 1916 |
| 3.39 | Seq ID No 1917 | Seq ID No 1918 | Seq ID No 1919 | Seq ID No 1920 |
| 3.40 | Seq ID No 1921 | Seq ID No 1922 | Seq ID No 1923 | Seq ID No 1924 |
| 3.41 | Seq ID No 1925 | Seq ID No 1926 | Seq ID No 1927 | Seq ID No 1928 |
| 3.42 | Seq ID No 1929 | Seq ID No 1930 | Seq ID No 1931 | Seq ID No 1932 |
| 3.43 | Seq ID No 1933 | Seq ID No 1934 | Seq ID No 1935 | Seq ID No 1936 |
| 3.44 | Seq ID No 1937 | Seq ID No 1938 | Seq ID No 1939 | Seq ID No 1940 |
| 3.45 | Seq ID No 1941 | Seq ID No 1942 | Seq ID No 1943 | Seq ID No 1944 |
| 3.46 | Seq ID No 1945 | Seq ID No 1946 | Seq ID No 1947 | Seq ID No 1948 |
| 3.47 | Seq ID No 1949 | Seq ID No 1950 | Seq ID No 1951 | Seq ID No 1952 |
| 3.48 | Seq ID No 1953 | Seq ID No 1954 | Seq ID No 1955 | Seq ID No 1956 |
| 3.49 | Seq ID No 1957 | Seq ID No 1958 | Seq ID No 1959 | Seq ID No 1960 |
| 3.50 | Seq ID No 1961 | Seq ID No 1962 | Seq ID No 1963 | Seq ID No 1964 |
| 3.51 | Seq ID No 1965 | Seq ID No 1966 | Seq ID No 1967 | Seq ID No 1968 |
| 3.52 | Seq ID No 1969 | Seq ID No 1970 | Seq ID No 1971 | Seq ID No 1972 |
| 3.53 | Seq ID No 1973 | Seq ID No 1974 | Seq ID No 1975 | Seq ID No 1976 |
| 3.54 | Seq ID No 1977 | Seq ID No 1978 | Seq ID No 1979 | Seq ID No 1980 |
| 3.55 | Seq ID No 1981 | Seq ID No 1982 | Seq ID No 1983 | Seq ID No 1984 |
| 3.56 | Seq ID No 1985 | Seq ID No 1986 | Seq ID No 1987 | Seq ID No 1988 |
| 3.57 | Seq ID No 1989 | Seq ID No 1990 | Seq ID No 1991 | Seq ID No 1992 |
| 3.58 | Seq ID No 1993 | Seq ID No 1994 | Seq ID No 1995 | Seq ID No 1996 |
| 3.59 | Seq ID No 1997 | Seq ID No 1998 | Seq ID No 1999 | Seq ID No 2000 |
| 3.60 | Seq ID No 2001 | Seq ID No 2002 | Seq ID No 2003 | Seq ID No 2004 |
| 3.61 | Seq ID No 2005 | Seq ID No 2006 | Seq ID No 2007 | Seq ID No 2008 |
| 3.62 | Seq ID No 2009 | Seq ID No 2010 | Seq ID No 2011 | Seq ID No 2012 |
| 3.63 | Seq ID No 2013 | Seq ID No 2014 | Seq ID No 2015 | Seq ID No 2016 |
| 3.64 | Seq ID No 2017 | Seq ID No 2018 | Seq ID No 2019 | Seq ID No 2020 |
| 3.65 | Seq ID No 2021 | Seq ID No 2022 | Seq ID No 2023 | Seq ID No 2024 |
| 3.66 | Seq ID No 2025 | Seq ID No 2026 | Seq ID No 2027 | Seq ID No 2028 |
| 3.67 | Seq ID No 2029 | Seq ID No 2030 | Seq ID No 2031 | Seq ID No 2032 |
| 3.68 | Seq ID No 2033 | Seq ID No 2034 | Seq ID No 2035 | Seq ID No 2036 |
| 3.69 | Seq ID No 2037 | Seq ID No 2038 | Seq ID No 2039 | Seq ID No 2040 |
| 3.70 | Seq ID No 2041 | Seq ID No 2042 | Seq ID No 2043 | Seq ID No 2044 |
| 3.71 | Seq ID No 2045 | Seq ID No 2046 | Seq ID No 2047 | Seq ID No 2048 |
| 3.72 | Seq ID No 2049 | Seq ID No 2050 | Seq ID No 2051 | Seq ID No 2052 |
| 3.73 | Seq ID No 2053 | Seq ID No 2054 | Seq ID No 2055 | Seq ID No 2056 |
| 3.74 | Seq ID No 2057 | Seq ID No 2058 | Seq ID No 2059 | Seq ID No 2060 |
| 3.75 | Seq ID No 2061 | Seq ID No 2062 | Seq ID No 2063 | Seq ID No 2064 |
| 3.76 | Seq ID No 2065 | Seq ID No 2066 | Seq ID No 2067 | Seq ID No 2068 |
| 3.77 | Seq ID No 2069 | Seq ID No 2070 | Seq ID No 2071 | Seq ID No 2072 |
| 3.78 | Seq ID No 2073 | Seq ID No 2074 | Seq ID No 2075 | Seq ID No 2076 |
| 3.79 | Seq ID No 2077 | Seq ID No 2078 | Seq ID No 2079 | Seq ID No 2080 |
| 3.80 | Seq ID No 2081 | Seq ID No 2082 | Seq ID No 2083 | Seq ID No 2084 |
| 3.81 | Seq ID No 2085 | Seq ID No 2086 | Seq ID No 2087 | Seq ID No 2088 |
| 3.82 | Seq ID No 2089 | Seq ID No 2090 | Seq ID No 2091 | Seq ID No 2092 |
| 3.83 | Seq ID No 2093 | Seq ID No 2094 | Seq ID No 2095 | Seq ID No 2096 |
| 3.84 | Seq ID No 2097 | Seq ID No 2098 | Seq ID No 2099 | Seq ID No 2100 |
| 3.85 | Seq ID No 2101 | Seq ID No 2102 | Seq ID No 2103 | Seq ID No 2104 |
| 3.86 | Seq ID No 2105 | Seq ID No 2106 | Seq ID No 2107 | Seq ID No 2108 |
| 3.87 | Seq ID No 2109 | Seq ID No 2110 | Seq ID No 2111 | Seq ID No 2112 |
| 3.88 | Seq ID No 2113 | Seq ID No 2114 | Seq ID No 2115 | Seq ID No 2116 |
| 3.89 | Seq ID No 2117 | Seq ID No 2118 | Seq ID No 2119 | Seq ID No 2120 |
| 3.90 | Seq ID No 2121 | Seq ID No 2122 | Seq ID No 2123 | Seq ID No 2124 |
| 3.91 | Seq ID No 2125 | Seq ID No 2126 | Seq ID No 2127 | Seq ID No 2128 |

In one aspect, the invention relates to a family 3-like binding molecule comprises a human V_{H} domain comprising a CDR3 sequence comprising SEQ ID NO. 1767 or a sequence with at least 70%, at least 80%, at least 90%, or at least 95% homology thereto.

In one embodiment, the family 3 or family 3-like binding molecule comprises at least one immunoglobulin single domain antibody directed against IL-17RA wherein said domain is a human V_{H} domain and wherein said V_{H} comprises at least one antigen binding site comprising a CDR3 sequence having SEQ ID NO. 1767 or a sequence with at least 70%, at least 80%, at least 90%, or at least 95% homology thereto. In one embodiment, sequence homology is at least 90%.

In one embodiment, the CDR3 sequence is selected from one of the CDR3 sequences as shown in table 3 with reference to FIG. 3. Thus, the CDR3 region comprises or consists of a sequence selected form SEQ ID Nos 1767, 1771, 1775, 1779, 1787, 1791, 1795, 1799, 1803, 1807, 1811, 1815, 1819, 1823, 1827, 1831, 1835, 1839, 1843, 1847, 1851, 1855, 1859, 1863, 1867, 1871, 1875, 1879, 1883, 1887, 1891, 1895, 1899, 1903, 1907, 1911, 1915, 1919, 1923, 1927, 1931, 1935, 1939, 1943, 1947, 1951, 1955, 1963, 1967, 1971, 1975, 1979, 1983, 1987, 1991, 1995, 1999, 2003, 2007, 2011, 2015, 2019, 2027, 2031, 2035, 2039, 2043, 2047, 2051, 2055, 2059, 2063, 2067, 2071, 2075, 2079, 2083, 2087, 2091, 2095, 2099, 2103, 2107, 2111, 2115, 2119, 2123 or 2127.

In one embodiment, the family 3-like sequence comprises at least one antigen binding site comprising hypervariable regions CDR1, CDR2 and CDR3, said CDR1 comprises or consists of the amino acid sequence SEQ ID NO. 1765 or a sequence with at least at least 70%, at least 80%, at least 90%, at least 95% homology thereto, said CDR2 comprises or consists of the amino acid sequence SEQ ID NO. 1766 or a sequence with at least 70%, at least 80%, at least 90%, at least 95% homology thereto, and said CDR3 comprises or consists of the amino acid sequence SEQ ID NO. 1767 or a sequence with at least 70%, at least 80%, at least 90%, at least 95% homology thereto. For example, the CDR sequence may be a sequence selected from those shown in FIG. 3.

In one embodiment, said CDR1 comprises or consists of the amino acid sequence as shown in SEQ ID NO. 1765 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR2 comprises or consists of the amino acid sequence as shown in SEQ ID NO. 1766 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR3 comprises or consists of the amino acid sequence as shown in SEQ ID NO. 1767 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto.

In one embodiment, CDR1 comprises or consists of one of the CDR1 amino acid sequence listed above in table 3 with reference to FIG. 3, CDR2 comprises or consists of one of the CDR2 amino acid sequence listed above in table 3 with reference to FIG. 3 and CDR3 comprises or consists of one of the CDR3 amino acid sequence listed above in table 3 with reference to FIG. 3. In one embodiment, the binding molecule has combinations of CDR1, CDR2 and CDR3 as shown for clones 3.1 to 3.91 in FIG. 3.

In one embodiment, the binding molecule comprises a set of CDR1, CDR2 and CDR3 sequences of a V_{H} sequence as shown for clones 3.1 to 3.91 in FIG. 3. In one embodiment, the binding molecule has a set of CDR1, CDR2 and CDR3 sequences of a V_{H} sequence as shown for clones 3.1 in FIG. 3.

In one embodiment, CDR1 is SEQ ID NO. 1765, CDR2 is SEQ ID No. 1766 and CDR3 is SEQ ID NO. 1767.

In one embodiment, the family 3 or family 3-like binding molecule has a V_{H} domain that comprises or consists of SEQ ID NO. 1768 or a sequence with at least 40%, 50%, 60%, 70%, 80%, 81%, 82%, 83%, 84%, 85%, 86%, 87%, 88%, 89%, 90%, 91%, 92%, 93%, 94%, 95% 96%, 97%, 98% or 99% homology thereto. CDR sequences of such sequences are shown in FIG. 3. For example, the V_{H} domain comprises or consists of one of the V_{H} amino acid sequences listed above for clones 3.1 to 3.91 in table 3 with reference to FIG. 3. In one embodiment, the V_{H} sequence is selected from 1768, 1772, 1776, 1780, 1784, 1788, 1792, 1796, 1800, 1804, 1808, 1812, 1816, 1820, 1824, 1828, 1832, 1836, 1840, 1844, 1848, 1852, 1856, 1860, 1864, 1868, 1876, 1880, 1884, 1888, 1892, 1896, 1900, 1904, 1908, 1912, 1916, 1920, 1924, 1928, 1932, 1936, 1940, 1944, 1948, 1956, 1964, 1968, 1972, 1976, 1980, 1984, 1988, 1992, 1996, 2000, 2004, 2008, 2012, 2016, 2020, 2024, 2028, 2032, 2036, 2040, 2044, 2048, 2052, 2056, 2060, 2064, 2068, 2072, 2084, 2088, 2092, 2096, 2100, 2104, 2108, 2112, 2116, 2120 or 2128.

In another embodiment, the V_{H} domain comprises a sequence selected from one of the sequences in the forgoing but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences. In one embodiment, the V_{H} domain comprises or consists of SEQ ID NO. 1767 or a sequence which comprises one or more amino acid substitutions, for example 1, 2, 3, 4, 5, 6, 7, 8, 9 or 10 amino acid substitutions.

In another aspect, the invention relates to a binding molecule comprising or consisting of a V_{H} domain as shown in SEQ ID NO. 1768 or a variant thereof comprising amino acid substitutions compared to SEQ ID NO. 1768 as follows: residue 1 is Q, residue 31 is S, residue 36 is S, T, residue 51 is M, residue 52 is K, residue 53 is H, E, residue 59 is Q, N, residue 99 is A, residue 100 is W, residue 101 is S, residue 102 is G and/or residue 106 is D.

The family 3 or family 3-like binding molecules have KD, Koff, KA, Kd and IC₅₀ values as further described herein and as shown in the examples.

In one aspect, the invention relates to a binding molecule capable of binding human IL-17RA comprising a human V_{H} domain wherein said V_{H} domain comprises a family 4 or family 4-like sequence.

In one embodiment, the binding molecule comprises or consists of at least one immunoglobulin single domain antibody directed against IL-17RA, preferably human IL-17RA, wherein said domain is a human V_{H} domain and wherein said V_{H} domain comprises a family 4 or family 4-like sequence. These include the V_{H} sequence of the parent clone (clone 4.1; Seq ID No 2132) or a part thereof, for example a CDR3 sequence, and V_{H} sequences of clones or parts thereof that are derived from the parent clone 4.1 through a process of optimization, for example as shown in FIG. 4. CDR sequences and full length sequences of clones in family 4 are numbered according to table 4 as shown below.

TABLE 4
Family 4 CDR sequences and V_{H} sequences that are within the scope
of the invention. Corresponding sequences are shown in FIG. 4.

| CLONE | CDR1 | CDR2 | CDR3 | FULL LENGTH |
|---|---|---|---|---|
| 4.1 | Seq ID No 2129 | Seq ID No 2130 | Seq ID No 2131 | Seq ID No 2132 |
| 4.2 | Seq ID No 2133 | Seq ID No 2134 | Seq ID No 2135 | Seq ID No 2136 |
| 4.3 | Seq ID No 2137 | Seq ID No 2138 | Seq ID No 2139 | Seq ID No 2140 |
| 4.4 | Seq ID No 2141 | Seq ID No 2142 | Seq ID No 2143 | Seq ID No 2144 |
| 4.5 | Seq ID No 2145 | Seq ID No 2146 | Seq ID No 2147 | Seq ID No 2148 |
| 4.6 | Seq ID No 2149 | Seq ID No 2150 | Seq ID No 2151 | Seq ID No 2152 |
| 4.7 | Seq ID No 2153 | Seq ID No 2154 | Seq ID No 2155 | Seq ID No 2156 |
| 4.8 | Seq ID No 2157 | Seq ID No 2158 | Seq ID No 2159 | Seq ID No 2160 |
| 4.9 | Seq ID No 2161 | Seq ID No 2162 | Seq ID No 2163 | Seq ID No 2164 |
| 4.10 | Seq ID No 2165 | Seq ID No 2166 | Seq ID No 2167 | Seq ID No 2168 |
| 4.11 | Seq ID No 2169 | Seq ID No 2170 | Seq ID No 2171 | Seq ID No 2172 |
| 4.12 | Seq ID No 2173 | Seq ID No 2174 | Seq ID No 2175 | Seq ID No 2176 |
| 4.13 | Seq ID No 2177 | Seq ID No 2178 | Seq ID No 2179 | Seq ID No 2180 |
| 4.14 | Seq ID No 2181 | Seq ID No 2182 | Seq ID No 2183 | Seq ID No 2184 |
| 4.15 | Seq ID No 2185 | Seq ID No 2186 | Seq ID No 2187 | Seq ID No 2188 |
| 4.16 | Seq ID No 2189 | Seq ID No 2190 | Seq ID No 2191 | Seq ID No 2192 |
| 4.17 | Seq ID No 2193 | Seq ID No 2194 | Seq ID No 2195 | Seq ID No 2196 |
| 4.18 | Seq ID No 2197 | Seq ID No 2198 | Seq ID No 2199 | Seq ID No 2200 |
| 4.19 | Seq ID No 2201 | Seq ID No 2202 | Seq ID No 2203 | Seq ID No 2204 |
| 4.20 | Seq ID No 2205 | Seq ID No 2206 | Seq ID No 2207 | Seq ID No 2208 |
| 4.21 | Seq ID No 2209 | Seq ID No 2210 | Seq ID No 2211 | Seq ID No 2212 |
| 4.22 | Seq ID No 2213 | Seq ID No 2214 | Seq ID No 2215 | Seq ID No 2216 |
| 4.23 | Seq ID No 2217 | Seq ID No 2218 | Seq ID No 2219 | Seq ID No 2220 |
| 4.24 | Seq ID No 2221 | Seq ID No 2222 | Seq ID No 2223 | Seq ID No 2224 |
| 4.25 | Seq ID No 2225 | Seq ID No 2226 | Seq ID No 2227 | Seq ID No 2228 |
| 4.26 | Seq ID No 2229 | Seq ID No 2230 | Seq ID No 2231 | Seq ID No 2232 |
| 4.27 | Seq ID No 2233 | Seq ID No 2234 | Seq ID No 2235 | Seq ID No 2236 |
| 4.28 | Seq ID No 2237 | Seq ID No 2238 | Seq ID No 2239 | Seq ID No 2240 |
| 4.29 | Seq ID No 2241 | Seq ID No 2242 | Seq ID No 2243 | Seq ID No 2244 |
| 4.30 | Seq ID No 2245 | Seq ID No 2246 | Seq ID No 2247 | Seq ID No 2248 |
| 4.31 | Seq ID No 2249 | Seq ID No 2250 | Seq ID No 2251 | Seq ID No 2252 |
| 4.32 | Seq ID No 2253 | Seq ID No 2254 | Seq ID No 2255 | Seq ID No 2256 |
| 4.33 | Seq ID No 2257 | Seq ID No 2258 | Seq ID No 2259 | Seq ID No 2260 |
| 4.34 | Seq ID No 2261 | Seq ID No 2262 | Seq ID No 2263 | Seq ID No 2264 |
| 4.35 | Seq ID No 2265 | Seq ID No 2266 | Seq ID No 2267 | Seq ID No 2268 |
| 4.36 | Seq ID No 2269 | Seq ID No 2270 | Seq ID No 2271 | Seq ID No 2272 |
| 4.37 | Seq ID No 2273 | Seq ID No 2274 | Seq ID No 2275 | Seq ID No 2276 |
| 4.38 | Seq ID No 2277 | Seq ID No 2278 | Seq ID No 2279 | Seq ID No 2280 |
| 4.39 | Seq ID No 2281 | Seq ID No 2282 | Seq ID No 2283 | Seq ID No 2284 |
| 4.40 | Seq ID No 2285 | Seq ID No 2286 | Seq ID No 2287 | Seq ID No 2288 |
| 4.41 | Seq ID No 2289 | Seq ID No 2290 | Seq ID No 2291 | Seq ID No 2292 |
| 4.42 | Seq ID No 2293 | Seq ID No 2294 | Seq ID No 2295 | Seq ID No 2296 |
| 4.43 | Seq ID No 2297 | Seq ID No 2298 | Seq ID No 2299 | Seq ID No 2300 |
| 4.44 | Seq ID No 2301 | Seq ID No 2302 | Seq ID No 2303 | Seq ID No 2304 |
| 4.45 | Seq ID No 2305 | Seq ID No 2306 | Seq ID No 2307 | Seq ID No 2308 |
| 4.46 | Seq ID No 2309 | Seq ID No 2310 | Seq ID No 2311 | Seq ID No 2312 |
| 4.47 | Seq ID No 2313 | Seq ID No 2314 | Seq ID No 2315 | Seq ID No 2316 |
| 4.48 | Seq ID No 2317 | Seq ID No 2318 | Seq ID No 2319 | Seq ID No 2320 |
| 4.49 | Seq ID No 2321 | Seq ID No 2322 | Seq ID No 2323 | Seq ID No 2324 |
| 4.50 | Seq ID No 2325 | Seq ID No 2326 | Seq ID No 2327 | Seq ID No 2328 |
| 4.51 | Seq ID No 2329 | Seq ID No 2330 | Seq ID No 2331 | Seq ID No 2332 |
| 4.52 | Seq ID No 2333 | Seq ID No 2334 | Seq ID No 2335 | Seq ID No 2336 |
| 4.53 | Seq ID No 2337 | Seq ID No 2338 | Seq ID No 2339 | Seq ID No 2340 |
| 4.54 | Seq ID No 2341 | Seq ID No 2342 | Seq ID No 2343 | Seq ID No 2344 |
| 4.55 | Seq ID No 2345 | Seq ID No 2346 | Seq ID No 2347 | Seq ID No 2348 |
| 4.56 | Seq ID No 2349 | Seq ID No 2350 | Seq ID No 2351 | Seq ID No 2352 |
| 4.57 | Seq ID No 2353 | Seq ID No 2354 | Seq ID No 2355 | Seq ID No 2356 |
| 4.58 | Seq ID No 2357 | Seq ID No 2358 | Seq ID No 2359 | Seq ID No 2360 |
| 4.59 | Seq ID No 2361 | Seq ID No 2362 | Seq ID No 2363 | Seq ID No 2364 |
| 4.60 | Seq ID No 2365 | Seq ID No 2366 | Seq ID No 2367 | Seq ID No 2368 |
| 4.61 | Seq ID No 2369 | Seq ID No 2370 | Seq ID No 2371 | Seq ID No 2372 |
| 4.62 | Seq ID No 2373 | Seq ID No 2374 | Seq ID No 2375 | Seq ID No 2376 |
| 4.63 | Seq ID No 2377 | Seq ID No 2378 | Seq ID No 2379 | Seq ID No 2380 |
| 4.64 | Seq ID No 2381 | Seq ID No 2382 | Seq ID No 2383 | Seq ID No 2384 |
| 4.65 | Seq ID No 2385 | Seq ID No 2386 | Seq ID No 2387 | Seq ID No 2388 |
| 4.66 | Seq ID No 2389 | Seq ID No 2390 | Seq ID No 2391 | Seq ID No 2392 |
| 4.67 | Seq ID No 2393 | Seq ID No 2394 | Seq ID No 2395 | Seq ID No 2396 |
| 4.68 | Seq ID No 2397 | Seq ID No 2398 | Seq ID No 2399 | Seq ID No 2400 |
| 4.69 | Seq ID No 2401 | Seq ID No 2402 | Seq ID No 2403 | Seq ID No 2404 |
| 4.70 | Seq ID No 2405 | Seq ID No 2406 | Seq ID No 2407 | Seq ID No 2408 |
| 4.71 | Seq ID No 2409 | Seq ID No 2410 | Seq ID No 2411 | Seq ID No 2412 |
| 4.72 | Seq ID No 2413 | Seq ID No 2414 | Seq ID No 2415 | Seq ID No 2416 |
| 4.73 | Seq ID No 2417 | Seq ID No 2418 | Seq ID No 2419 | Seq ID No 2420 |
| 4.74 | Seq ID No 2421 | Seq ID No 2422 | Seq ID No 2423 | Seq ID No 2424 |
| 4.75 | Seq ID No 2425 | Seq ID No 2426 | Seq ID No 2427 | Seq ID No 2428 |
| 4.76 | Seq ID No 2429 | Seq ID No 2430 | Seq ID No 2431 | Seq ID No 2432 |
| 4.77 | Seq ID No 2433 | Seq ID No 2434 | Seq ID No 2435 | Seq ID No 2436 |
| 4.78 | Seq ID No 2437 | Seq ID No 2438 | Seq ID No 2439 | Seq ID No 2440 |
| 4.79 | Seq ID No 2441 | Seq ID No 2442 | Seq ID No 2443 | Seq ID No 2444 |
| 4.80 | Seq ID No 2445 | Seq ID No 2446 | Seq ID No 2447 | Seq ID No 2448 |
| 4.81 | Seq ID No 2449 | Seq ID No 2450 | Seq ID No 2451 | Seq ID No 2452 |
| 4.82 | Seq ID No 2453 | Seq ID No 2454 | Seq ID No 2455 | Seq ID No 2456 |
| 4.83 | Seq ID No 2457 | Seq ID No 2458 | Seq ID No 2459 | Seq ID No 2460 |
| 4.84 | Seq ID No 2461 | Seq ID No 2462 | Seq ID No 2463 | Seq ID No 2464 |
| 4.85 | Seq ID No 2465 | Seq ID No 2466 | Seq ID No 2467 | Seq ID No 2468 |
| 4.86 | Seq ID No 2469 | Seq ID No 2470 | Seq ID No 2471 | Seq ID No 2472 |
| 4.87 | Seq ID No 2473 | Seq ID No 2474 | Seq ID No 2475 | Seq ID No 2476 |
| 4.88 | Seq ID No 2477 | Seq ID No 2478 | Seq ID No 2479 | Seq ID No 2480 |
| 4.89 | Seq ID No 2481 | Seq ID No 2482 | Seq ID No 2483 | Seq ID No 2484 |
| 4.90 | Seq ID No 2485 | Seq ID No 2486 | Seq ID No 2487 | Seq ID No 2488 |
| 4.91 | Seq ID No 2489 | Seq ID No 2490 | Seq ID No 2491 | Seq ID No 2492 |
| 4.92 | Seq ID No 2493 | Seq ID No 2494 | Seq ID No 2495 | Seq ID No 2496 |
| 4.93 | Seq ID No 2497 | Seq ID No 2498 | Seq ID No 2499 | Seq ID No 2500 |
| 4.94 | Seq ID No 2501 | Seq ID No 2502 | Seq ID No 2503 | Seq ID No 2504 |
| 4.95 | Seq ID No 2505 | Seq ID No 2506 | Seq ID No 2507 | Seq ID No 2508 |
| 4.96 | Seq ID No 2509 | Seq ID No 2510 | Seq ID No 2511 | Seq ID No 2512 |
| 4.97 | Seq ID No 2513 | Seq ID No 2514 | Seq ID No 2515 | Seq ID No 2516 |
| 4.98 | Seq ID No 2517 | Seq ID No 2518 | Seq ID No 2519 | Seq ID No 2520 |
| 4.99 | Seq ID No 2521 | Seq ID No 2522 | Seq ID No 2523 | Seq ID No 2524 |
| 4.100 | Seq ID No 2525 | Seq ID No 2526 | Seq ID No 2527 | Seq ID No 2528 |
| 4.101 | Seq ID No 2529 | Seq ID No 2530 | Seq ID No 2531 | Seq ID No 2532 |
| 4.102 | Seq ID No 2533 | Seq ID No 2534 | Seq ID No 2535 | Seq ID No 2536 |
| 4.103 | Seq ID No 2537 | Seq ID No 2538 | Seq ID No 2539 | Seq ID No 2540 |
| 4.104 | Seq ID No 2541 | Seq ID No 2542 | Seq ID No 2543 | Seq ID No 2544 |
| 4.105 | Seq ID No 2545 | Seq ID No 2546 | Seq ID No 2547 | Seq ID No 2548 |
| 4.106 | Seq ID No 2549 | Seq ID No 2550 | Seq ID No 2551 | Seq ID No 2552 |
| 4.107 | Seq ID No 2553 | Seq ID No 2554 | Seq ID No 2555 | Seq ID No 2556 |

In one aspect of the invention, the family 4 or family 4-like binding molecule comprises a human V_{H} domain comprising a hypervariable region CDR3 said CDR3 having the amino acid sequence SEQ ID NO. 2131, or a sequence having at least 70%, at least 80%, at least 90%, or at least 95% homology to SEQ ID NO. 2131.

In one embodiment, the family 4 or family 4-like binding molecule comprises a binding molecule comprising or consisting of at least one immunoglobulin single domain antibody directed against IL-17R wherein said domain is a human V_{H} domain and wherein said V_{H} domain comprises hypervariable region CDR3 said CDR3 having the amino acid sequence SEQ ID NO. 2131, or a sequence having at least 70%, at least 80%, at least 90%, or at least 95% homology thereto. In one embodiment, sequence homology is at least 90%.

In one embodiment, the CDR3 region is selected from one of the CDR3 sequence as shown in table 4 with reference to FIG. 4. Thus, the CDR3 sequence is selected from SEQ ID Nos: 2131, 2135, 2139, 2143, 2147, 2151, 2155, 2159, 2163, 2167, 2171, 2175, 2179, 2183, 2187, 2191, 2195, 2199, 2203, 2207, 2211, 2215, 2219, 2223, 2227, 2231, 2235, 2239, 2243, 2247, 2251, 2255, 2259, 2263, 2267, 2271, 2275, 2279, 2283, 2287, 2291, 2295, 2299, 2303, 2307, 2311, 2315, 2319, 2323, 2327, 2331, 2335, 2339, 2343, 2347, 2351, 2355, 2359, 2363, 2367, 2371, 2375, 2379, 2383, 2387, 2391, 2395, 2399, 2403, 2407, 2411, 2415, 2419, 2423, 2427, 2435, 2439, 2443, 2447, 2451, 2455, 2459, 2463, 2467, 2471, 2475, 2479, 2483, 2487, 2491, 2495, 2499, 2503, 2507, 2511, 2515, 2519, 2523, 2527, 2531, 2535, 2539, 2543, 2551 or 2555.

In one embodiment, the family 4 or family 4-like binding molecule comprises at least one antigen binding site comprising hypervariable regions CDR1, CDR2 and CDR3, said CDR1 comprises or consists of the amino acid sequence SEQ ID NO. 2129 or a sequence with at least 70%, at least 80%, at least 90%, or at least 95% homology thereto, said CDR2 comprises or consists of the amino acid sequence SEQ ID NO. 2130 or a sequence with at least 70%, at least 80%, at least 90%, or at least 95% homology thereto, and said CDR3 comprises or consists of the amino acid sequence SEQ ID NO. 2131, or a sequence with at least 70%, at least 80%, at least 90%, or at least 95% homology thereto. For example, the CDR sequence may be a CDR sequence selected form those shown in FIG. 4.

In one embodiment, said CDR1 comprises or consists of the amino acid sequence as shown in SEQ ID NO. 2129 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR2 comprises or consists of the amino acid sequence as shown in SEQ ID NO. 2130 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR3 comprises or consists of the amino acid sequence as shown in SEQ ID NO. 2131 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto.

In one embodiment, CDR1 comprises or consists of one of the CDR1 amino acid sequence listed above in table 4 with reference to FIG. 4, CDR2 comprises or consists of one of the CDR2 amino acid sequence listed above in table 4 with reference to FIG. 4 and CDR3 comprises or consists of one of the CDR3 amino acid sequence listed above in table 4 with reference to FIG. 4. In one embodiment, the binding molecule has combinations of CDR1, CDR2 and CDR3 as shown for clones 4.1 to 4.107 in FIG. 4. In one embodiment, the binding molecule comprises a set of CDR1, CDR2 and CDR2 sequences of a V_{H} sequence as shown for clones 4.1 to 4.107 in FIG. 4. In one embodiment, the binding molecule has a set of CDR1, CDR2 and CDR2 sequences of a V_{H} sequence as shown for clone 4.1 in FIG. 4.

In one embodiment, the family 4 or family 4-like binding molecule has a V_{H} domain that comprises or consists of SEQ ID NO. 2132 or a sequence with at least 50%, 60%, 70%, 80%, 81%, 82%, 83%, 84%, 85%, 87%, 88%, 89%, 90%, 91%, 92%, 93%, 94%, 96%, 97%, 98%, 99% homology thereto. CDR sequences of such V_{H} sequences are shown in FIG. 4. For example, the V_{H} domain comprises or consists of one of the V_{H} amino acid sequences listed above for clones 41.1 to 4.107 in FIG. 4 and table 4. Thus, the V_{H} sequence comprises or consists of a V_{H} sequence selected from SEQ ID NOs. 2132, 2136, 2140, 2144, 2148, 2152, 2156, 2160, 2164, 2168, 2172, 2184, 2188, 2192, 2196, 2200, 2204, 2208, 2212, 2216, 2220, 2224, 2228, 2232, 2236, 2240, 2244, 2248, 2252, 2256, 2260, 2264, 2268, 2272, 2276, 2280, 2284, 2288, 2292, 2300, 2304, 2308, 2312, 2316, 2320, 2324, 2328, 2332, 2336, 2340, 2344, 2348, 2352, 2356, 2360, 2364, 2368, 2372, 2376, 2380, 2384, 2388, 2392, 2396, 2400, 2404, 2408, 2412, 2416, 2420, 2424, 2428, 2432, 2436, 2440, 2444, 2452, 2456, 2460, 2468, 2472, 2476, 2480, 2484, 2488, 2492, 2496, 2500, 2504, 2508, 2512, 2516, 2520, 2524, 2528, 2532, 2536, 2540, 2544, 2548, 2552 or 2556.

In another embodiment, the V_{H} domain comprises a sequence selected from one of the sequences in the forgoing, but comprises 1, 2, 3, 4, 5, 6, 7, 8, 9 or 10 amino acid substitutions. In one embodiment, these are in the framework region. In another embodiment, these are in the CDR. In one embodiment, the amino acid substitutions are in the framework and CDR sequences. In one embodiment, the V_{H} domain comprises or consists of SEQ ID NO. 2132 or a sequence which comprises one or more amino acid substitutions, for example 1, 2, 3, 4, 5, 6, 7, 8, 9 or 10 amino acid substitutions.

In another aspect, the invention relates to a binding molecule comprising or consisting of a V_{H} domain as shown in SEQ ID NO. 2132 or a variant thereof comprising amino acid substitutions compared to SEQ ID NO. 2132 as follows residue 5 is Q, residue 10 is G, residue 28 is T, residue 31 is S, G, residue 32 is H, residue 33 is I, G, V, residue 37 is V, M, residue 51 is I, residue 54 is N, K, E, residue 55 is N, residue 61 is S, T, residue 66 is D, residue 100 is D, residue 101 is S, F, residue 102 is G, residue 103 is S and/or residue 106 is Q, T.

The family 4 or family 4-like binding molecules have KD, Koff, KA, Kd and IC₅₀ values as further described herein and as shown in the examples.

In one aspect, the invention relates to a binding molecule capable of binding human IL-17RA comprising a human V_{H} domain wherein said V_{H} domain comprises a family 5 or family 5-like sequence.

In one embodiment, the binding molecule comprises or consists of at least one immunoglobulin single domain antibody directed against IL-17RA, preferably human IL-17RA, wherein said domain is a human V_{H} domain and wherein said V_{H} domain comprises a family 5 or family 5-like sequence. These include the V_{H} sequence of the parent clone (clone 5.1; SEQ ID NO. 2560) or a part thereof, for example a CDR3 sequence, and V_{H} sequences of clones that are derived from the parent clone 5.1 through a process of optimization, for example as shown in FIG. 5.

CDR sequences and full length sequences of clones in family 5 are numbered according to table 5 as shown below.

TABLE 5
Family 5 CDR sequences and V_{H} sequences that are within the scope
of the invention. Corresponding sequences are shown in FIG. 5.

| Clone | | | | Full length VH |
|---|---|---|---|---|
| name | CDR1 SEQ ID NO. | CDR2 SEQ ID NO. | CDR3 SEQ ID NO. | sequence SEQ ID NO. |
| 5.1 | SEQ ID NO. 2557 | SEQ ID NO. 2558 | SEQ ID NO. 2559 | SEQ ID NO. 2560 |
| 5.2 | SEQ ID NO. 2561 | SEQ ID NO. 2562 | SEQ ID NO. 2563 | SEQ ID NO. 2564 |
| 5.3 | SEQ ID NO. 2565 | SEQ ID NO. 2566 | SEQ ID NO. 2567 | SEQ ID NO. 2568 |
| 5.4 | SEQ ID NO. 2569 | SEQ ID NO. 2570 | SEQ ID NO. 2571 | SEQ ID NO. 2572 |

In one aspect of the invention, the family 5 or family 5-like binding molecule comprises a human V_{H} domain comprising a CDR3 sequence comprising amino acid sequence SEQ ID NO. 2559, or a sequence having at least 70%, at least 80%, at least 90%, or at least 95% homology thereto.

In one embodiment, the family 5 or family 5-like binding molecule comprises at least one immunoglobulin single domain antibody directed against IL-17R wherein said domain is a human V_{H} domain and wherein said V_{H} comprises at least one antigen binding site comprising hypervariable region CDR3 said CDR3 having the amino acid sequence SEQ ID NO. 2559, or a sequence having at least 70%, at least 80%, at least 90%, or at least 95% homology thereto.

In one embodiment, the homology is at least 90%.

In one embodiment, the CDR3 sequence is selected from one of the CDR3 sequences as shown in table 5 with reference to FIG. 5. Thus, the CDR3 sequence is selected from 2559, 2563, 2567 or 2571.

In one embodiment, the family 5 or family 5-like sequence comprises a binding molecule comprising hypervariable regions CDR1, CDR2 and CDR3, said CDR1 comprises or consists of the amino acid sequence SEQ ID NO. 2557 or a sequence with at least 70%, at least 80%, at least 90%, or at least 95% homology thereto, said CDR2 comprises or consists of the amino acid sequence SEQ ID NO. 2558 or a sequence with at least 70%, at least 80%, at least 90%, or at least 95% homology thereto, and said CDR3 comprises or consists of the amino acid sequence SEQ ID NO. 2559, or a sequence with at least 70%, at least 80%, at least 90%, or at least 95% homology thereto. For example, the CDR sequence may be a CDR sequence selected from those shown in FIG. 5.

In one embodiment, said CDR1 comprises or consists of the amino acid sequence as shown in SEQ ID NO. 2557 or a sequence with at least 70%, 80%, 81%, 82%, 83%, 84%, 85%, 87%, 88%, 89%, 90%, 91%, 92%, 93%, 94%, 96%, 97%, 98%, 99% homology thereto. In one embodiment, said CDR2 comprises or consists of the amino acid sequence as shown in SEQ ID NO. 2558 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR3 comprises or consists of the amino acid sequence as shown in SEQ ID NO. 2559 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto.

In one embodiment, CDR1 comprises or consists of one of the CDR1 amino acid sequence listed above in table 5 with reference to FIG. 5, CDR2 comprises or consists of one of the CDR2 amino acid sequence listed above in table 5 with reference to FIG. 5 and CDR3 comprises or consists of one of the CDR3 amino acid sequence listed above in table 5 with reference to FIG. 5. In one embodiment, the binding molecule has combinations of CDR1, CDR2 and CDR3 as shown for clones 5.1 to 5.4 in FIG. 5. In one embodiment, the binding molecule comprises a set of CDR1, CDR2 and CDR3 sequences of a V_{H} sequence as shown for clones 5.1 to 5.4 in FIG. 5.

In one embodiment, the family 5 or family 5-like binding molecule has a V_{H} domain that comprises or consists of SEQ ID NO. 2560 or a sequence with at least 40%, 50%, 60%, 70%, 80%, 81%, 82%, 83%, 84%, 85%, 86%, 87%, 88%, 89%, 90%, 91%, 92%, 93%, 94%, 95% 96%, 97%, 98% or 99% homology thereto. Thus, the V_{H} comprises or consist of a V_{H} selected form SEQ ID Nos. 2560, 2564, 2568 or 2572. In another embodiment, the V_{H} domain comprises a sequence selected from one of the sequences in the forgoing but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences. In one embodiment, the V_{H} domain comprises or consists of SEQ ID NO. 2560 or a sequence which comprises one or more amino acid substitutions, for example 1, 2, 3, 4, 5, 6, 7, 8, 9 or 10 amino acid substitutions.

In another aspect, the invention relates to a binding molecule comprising or consisting of a V_{H} domain as shown in SEQ ID NO. 2132 or a variant thereof comprising amino acid substitutions compared to SEQ ID NO. 2132 as follows residue 1 is Q, residue 32 is Y, residue 33 is Y, residue 52 is G, residue 53 is G, residue 56 is D, residue 57 is V, residue 103 is H, residue 104 is D and/or residue 106 is K.

The family 5 or family 5-like binding molecules have KD, Koff, KA, Kd and IC₅₀ values as further described herein and as shown in the examples.

In another aspect, the invention relates to a binding molecule capable of binding human IL-17RA comprising a human V_{H} domain wherein said V_{H} domain comprises a family 6 or family 6-like sequence.

In one embodiment, the binding molecule comprises or consists of at least one immunoglobulin single domain antibody directed against IL-17RA, preferably human IL-17RA, wherein said domain is a human v domain and wherein said V_{H} domain comprises a family 6 or family 6-like sequence. These include the V_{H} sequence of the parent clone (clone 6.1; SEQ ID NO. 2576) or a part thereof, for example a CDR3 sequence, and V_{H} sequences of clones or that are derived from the parent clone 6.1 through a process of optimization, for example as shown in FIG. 6. CDR sequences and full length sequences of clones in family 6 are numbered according to table 6 as shown below.

TABLE 6
Family 6 CDR sequences and V_{H} sequences that are within the scope
of the invention. Corresponding sequences are shown in FIG. 6.

| Clone | | | | Full length |
|---|---|---|---|---|
| name | CDR1 | CDR2 | CDR3 | sequence |
| 6.1 | SEQ ID NO. | SEQ ID NO. | SEQ ID NO. | SEQ ID NO. |
| | 2573 | 2574 | 2575 | 2576 |

In one aspect, the invention relates to a family 6 or family 6-like binding molecule comprises a human V_{H} domain comprising a CDR3 sequence CDR3 comprising the amino acid sequence SEQ ID NO. 2575, or a sequence having at least 70%, at least 80%, at least 90%, or at least 95% homology thereto.

In one embodiment, the family 6 or family 6-like binding molecule comprises at least one immunoglobulin single domain antibody directed against IL-17R comprising at least one antigen binding site comprising hypervariable region CDR3 said CDR3 having the amino acid sequence SEQ ID NO. 2575, or a sequence having at least 70%, at least 80%, at least 90%, or at least 95% homology thereto. In one embodiment, the homology is at least 90%.

In one embodiment, the family 6 or family 6-like binding molecule comprises hypervariable regions CDR1, CDR2 and CDR3, said CDR1 comprises or consists of the amino acid sequence SEQ ID NO. 2573 or a sequence with at least 70%, at least 80%, at least 90%, or at least 95% homology thereto, said CDR2 comprises or consists of the amino acid sequence SEQ ID NO. 2574 or a sequence with at least 70%, at least 80%, at least 90%, or at least 95% homology thereto, and said CDR3 comprises or consists of the amino acid sequence SEQ ID NO. 2575, or a sequence with at least 80%, at least 90%, or at least 95% homology thereto.

In one embodiment, said CDR1 comprises or consists of the amino acid sequence SEQ ID NO. 2573 or a sequence with at least 70%, 80%, 81%, 82%, 83%, 84%, 85%, 86%, 87%, 88%, 89%, 90%, 91%, 92%, 93%, 94%, 95% 96%, 97%, 98% or 99% homology thereto. In one embodiment, said CDR2 comprises or consists of the amino acid sequence SEQ ID NO. 2574 or a sequence with at least 70%, 80%, 81%, 82%, 83%, 84%, 85%, 86%, 87%, 88%, 89%, 90%, 91%, 92%, 93%, 94%, 95%, 96%, 97%, 98% or 99% homology thereto. In one embodiment, said CDR3 comprises or consists of the amino acid sequence SEQ ID NO. 2575 or a sequence with at least 70%, 80%, 81%, 82%, 83%, 84%, 85%, 86%, 87%, 88%, 89%, 90%, 91%, 92%, 93%, 94%, 95%, 96%, 97%, 98% or 99% homology thereto.

In one embodiment, the family 6 or family 6-like binding molecule has a V_{H} domain that comprises or consists of SEQ ID NO. 2576 or a sequence with at least 40%, 50%, 60%, 70%, 80%, 80%, 81%, 82%, 83%, 84%, 85%, 86%, 87%, 88%, 89%, 90%, 91%, 92%, 93%, 94%, 95% 96%, 97%, 98% or 99% homology thereto.

In another embodiment, the V_{H} domain comprises SEQ ID NO. 2576, but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences.

The family 6 or family 6-like binding molecules have KD, Koff, KA, Kd and IC₅₀ values as further described herein and as shown in the examples.

In another aspect, the invention relates to a binding molecule capable of binding human IL-17RA comprising a human V_{H} domain wherein said V_{H} domain comprises a family 7 or family 7-like sequence.

In one embodiment, the binding molecule comprises or consists of at least one immunoglobulin single domain antibody directed against IL-17RA, preferably human IL-17RA, wherein said domain is a human V_{H} domain and wherein said V_{H} domain comprises a family 7 or family 7-like sequence. These include the V_{H} sequence of the parent clone (clone 7.1; SEQ ID NO. 2580) or a part thereof, for example a CDR3 sequence, and V_{H} sequences of clones or parts thereof that are derived from the parent clone 7.1 through a process of optimization, for example as shown in FIG. 7. CDR sequences and full length sequences of clones in family 7 are numbered according to table 7 as shown below.

TABLE 7
Family 7 CDR sequences and V_{H} sequences that are within the scope
of the invention. Corresponding sequences are shown in FIG. 7.

| Clone | | | | Full length |
|---|---|---|---|---|
| name | CDR1 | CDR2 | CDR3 | sequence |
| 7.1 | SEQ ID NO. | SEQ ID NO. | SEQ ID NO. | SEQ ID NO. |
| | 2577 | 2578 | 2579 | 2580 |

In one aspect, the invention relates to a family 7 or family 7-like binding molecule comprises a human V_{H} domain comprising a CDR3 sequence CDR3 comprising the amino acid sequence SEQ ID NO. 2579, or a sequence having at least 70%, at least 80%, at least 90%, or at least 95% homology thereto.

In one embodiment, the family 7 or family 7-like binding molecule comprises at least one immunoglobulin single domain antibody directed against IL-17R comprising a human V_{H} domain comprising at least one antigen binding site comprising CDR1, CDR2 and CDR3, said CDR1 comprises or consists of the amino acid sequence SEQ ID NO. 2577 or a sequence with at least 70%, at least 80%, at least 90%, or at least 95% homology thereto, said CDR2 comprises or consists of the amino acid sequence SEQ ID NO. 2578 or a sequence with at least 70%, at least 80%, at least 90%, or at least 95% homology thereto, and said CDR3 comprises or consists of the amino acid sequence SEQ ID NO. 2579, or a sequence with at least 70%, at least 80%, at least 90%, or at least 95% homology thereto.

In one embodiment, said CDR1 comprises or consists of the amino acid sequence SEQ ID NO. 2577 or a sequence with at least 70%, 80%, 81%, 82%, 83%, 84%, 85%, 86%, 87%, 88%, 89%, 90%, 91%, 92%, 93%, 94%, 95%, 96%, 97%, 98% or 99% homology thereto. In one embodiment, said CDR2 comprises or consists of the amino acid sequence SEQ ID NO. 2578 or a sequence with at least 70%, 80%, 81%, 82%, 83%, 84%, 85%, 86%, 87%, 88%, 89%, 90%, 91%, 92%, 93%, 94%, 95%, 96%, 97%, 98% or 99% homology thereto. In one embodiment, said CDR3 comprises or consists of the amino acid sequence SEQ ID NO. 2579 or a sequence with at least 70%, 80%, 81%, 82%, 83%, 84%, 85%, 86%, 87%, 88%, 89%, 90%, 91%, 92%, 93%, 94%, 95%, 96%, 97%, 98% or 99% homology thereto.

In one embodiment, the family 7 or family 7-like V_{H} has a V_{H} domain that comprises or consists of SEQ ID NO. 2580 or a sequence with at least 40%, 50%, 60%, 70%, 80%, 80%, 81%, 82%, 83%, 84%, 85%, 86%, 87%, 88%, 89%, 90%, 91%, 92%, 93%, 94%, 95%, 96%, 97%, 98% or 99% homology thereto.

In another embodiment, the V_{H} domain comprises SEQ ID NO. 2580, but comprises 1, 2, 3, 4, 5, 6, 7, 8, 9 or 10 amino acid substitutions. In one embodiment, these are in the framework region. In another embodiment, these are in the CDRs. In one embodiment, the amino acid substitutions are in the framework and CDR sequences.

The family 7 or family 7-like binding molecules have KD, Koff, KA, Kd and IC₅₀ values as further described herein and as shown in the examples.

In one aspect, the binding molecule according to the invention comprises a CDR3 sequence selected from a family 1 or family 1-like, family 2 or family 2-like, family 3 or family 3-like, family 4 or family 4-like, family 5 or family 5-like, family 6 or family 6-like or family 7 or family 7-like CDR3 sequence combined with a CDR1 and CDR2 sequence from another family listed herein.

For example, the binding molecule according to the invention comprises a family 1 or family 1-like CDR3 sequence combined with a CDR1 and/or a CDR2 sequence from one or two other families as shown in Table 2, 3, 4, 5, 6 or 7.

In another aspect, the binding molecule according to the invention comprises a family 2 or family 2-like CDR3 sequence combined with a CDR1 and/or a CDR2 sequence from one or two other families as shown in Table 1, 3, 4, 5, 6 or 7. Various combinations are possible as would be appreciated by a skilled person.

In another aspect, the binding molecule according to the invention comprises a family 3 or family 3-like CDR3 sequence combined with a CDR1 and/or a CDR2 sequence from one or two other families as shown in Table 1, 2, 4, 5, 6 or 7. Various combinations are possible as would be appreciated by a skilled person.

In another aspect, the binding molecule according to the invention comprises a family 4 or family 4-like CDR3 sequence combined with a CDR1 and/or a CDR2 sequence from one or two other families as shown in Table 1, 2, 3, 5, 6 or 7. Various combinations are possible as would be appreciated by a skilled person.

In another aspect, the binding molecule according to the invention comprises a family 5 or family 5-like CDR3 sequence combined with a CDR1 and/or a CDR2 sequence from one or two other families as shown in Table 1, 2, 3, 4, 6 or 7. Various combinations are possible as would be appreciated by a skilled person.

In another aspect, the binding molecule according to the invention comprises a family 6 or family 6-like CDR3 sequence combined with a CDR1 and/or a CDR2 sequence from one or two other families as shown in Table 1, 2, 3, 5 or 7. Various combinations are possible as would be appreciated by a skilled person.

In another aspect, the binding molecule according to the invention comprises a family 7 or family 7-like CDR3 sequence combined with a CDR1 and/or a CDR2 sequence from one or two other families as shown in Table 1, 2, 3, 5 or 6. Various combinations are possible as would be appreciated by a skilled person.

As mentioned above, also within the scope of the invention are V_{H} domains that comprise additional C or N terminal residues, for example linker residues introduced from the expression vector used (e.g., LEGGGS from phagemid vector or AA) and/or His tags, e.g. hexa-His (HHHHHH, SEQ ID NO: 2605).

A binding molecule described herein may be provided as a fusion protein with one or more additional protein moiety. For example, the binding molecule described herein may be provided as a fusion with a second moiety.

The second moiety may comprise a V_{H} domain that is also specific for human IL-17RA thus providing a bivalent binding molecule. In one embodiment, the binding molecule is biparatopic. Biparatopic binding molecules bind to different epitopes. Biparatopic binding molecules of the present invention can be constructed using methods known art.

For example, a family 1 binding molecule may be linked to a family 2, 3, 4, 5, 6 or 7 or family 2, 3, 4, 5, 6 or 7-like binding molecule.

In another embodiment, the second moiety comprises a V_{H} domain or another antibody fragment that is specific for a different antigen to provide a bispecific binding molecule. As used herein, the term “bispecific binding molecule” thus refers to a polypeptide that comprises a binding molecule as described herein which has a binding site that has binding specificity for IL17-RA, and a second polypeptide domain which has a binding site that has binding specificity for a second target, i.e., the agent has specificity for two targets. The first target and the second target are not the same, i.e. are different targets e.g., proteins, but are both present on a cell. Accordingly, a bispecific binding molecule as described herein can selectively and specifically bind to a cell that expresses (or displays on its cell surface) the first target and the second target. In another embodiment, the binding molecule comprises more than two moieties.

In another embodiment, more than two moieties are joined together providing a multispecific binding molecule. A multispecific polypeptide agent as described herein can in addition bind one or more additional targets, i.e., a multispecific polypeptide can bind at least two, at least three, at least four, at least five, at least six, or more targets, wherein the multispecific polypeptide agent has at least two, at least, at least three, at least four, at least five, at least six, or more target binding sites respectively.

As used herein, the term “target” refers to a biological molecule (e.g., peptide, polypeptide, protein, lipid, carbohydrate) to which a polypeptide domain which has a binding site can selectively bind. The target can be, for example, an intracellular target (e.g. an intracellular protein target) or a cell surface target (e.g., a membrane protein, a receptor protein). Preferably, a target is a cell surface target, such as a cell surface protein. Preferably, the first cell surface target and second cell surface target are both present on a cell.

Multispecific antibodies of the present invention can be constructed using methods known art.

In biparatopic or multispecific binding molecules, the moieties are joined by a linker, for example a polypeptide linker. Suitable linkers, for example comprising linker including GS residues such as (Gly₄Ser)ₙ, where n=from 1 to 10, e.g. 1, 2, 3, 4, 5, 6, 7, 8, 9 or 10 are known in the art.

If desired, bispecific or multispecific binding molecules can be linked to an antibody Fc region or a fragment thereof, comprising one or both of C_{H}2 and C_{H}3 domains, and optionally a hinge region. For example, vectors encoding bispecific or multispecific binding molecules linked as a single nucleotide sequence to an Fc region or a fragment thereof can be used to prepare such polypeptides.

Exemplary second antigen targets include leukocyte receptors, e.g., MHC, CD2, CD3, CD4, CD7, CD8, CD25, CD28, CD4, CD45, CD58, CD80, CD86 or their ligands; TNF, IL-1 IL-15, IL-23, IL-6 or CD20. This list is not limited to the agents mentioned.

In one embodiment, the second moiety may serve to prolong the half-life of the binding molecule. The second moiety or third may comprise a protein that binds a serum albumin, e.g., human serum albumin (HSA). The second moiety may comprise a V_{H} domain that binds serum albumin, e.g. human serum albumin (HSA). The second moiety may comprise a serum albumin, e.g. a human serum albumin (HSA) or a variant thereof such as C34S. Further provided is binding molecule as described herein comprising a V_{H} domain and an Fc domain or a fragment thereof, e.g., wherein the V_{H} domain is fused to an Fc domain or a fragment thereof. Further provided is a binding molecule that comprises a second variable domain that specifically binds a second antigen, where the second antigen is an antigen other than human IL-17R. The second antigen may be a cluster of differentiation (CD) molecule or a Major Histocompatibility Complex (MHC) Class II molecule.

The present invention further provides an isolated nucleic acid encoding a binding member of the present invention. Nucleic acid may include DNA and/or RNA. In one aspect, the present invention provides a nucleic acid that codes for a CDR, for example a CDR3, or set of CDRs, a V_{H} domain or binding molecule as defined above. In one aspect, the invention also relates to nucleic acid sequences encoding V_{H} domains of family 1, 2, 3, 4, 5, 6 or 7 as shown herein. Examples of such sequences encoding V_{H} sequences of specific clones are shown below.

1.1
SEQ ID NO. 2581
CAGGTGCAGCTGGTGGAGTCTGGGGGAGGCTTGGTACAGCCTGGCAG
GTCCCTGAGACTCTCCTGTGCAGCCTCTGGATTCACCTTTGATGATT
ATGCCATGCACTGGGTCCGGCAAGCTCCAGGGAAGGGCCTGGAGTGG
GTCTCAGGTATTAGTTGGAATAGTGGTAGGATGGACTATGCGGACTC
TGTGAAGGGCCGATTCACCATCTCCAGAGACAACGCCAAGAAGTCCC
TGTATCTGCAAATGAACAGTCTGAGAGCTGAGGACACGGCCATGTAT
TACTGTGCAAAAGAGAAGGGCCTAGGATTTTGTCGTGGTGGTAGCTG
TTCCTACTTTGACTATAGGGGCCAGGGAACCCTGGTCACCGTCTCCT
CA
1.2
SEQ ID NO. 2582
GAGGTGCAGCTGGTGGAGTCTGGGGGAGGCTTGGTACAGCCTGGCAG
GTCCCTGAGACTCTCCTGTGCAGCCTCTGGATTCACCTTTGATGATT
ATGCCATGCACTGGGTCCGACAAGCTCCAGGAAAGGGCCTGGAGTGG
GTCTCAGGTATTAGTTGGAATAGTGGTAGGATGGACTATGCGGACTC
TGTGAAGGGCCGATTCACCATCTCCAGAGACAACGCCAAGAACTCCC
TGTATCTGCAAATGAACAGTCTGAGAGCTGAGGACACGGCCTTATAT
TACTGTGCAAAGGAGAAGGGCCTAGGATATTGTCGTGGTGGTAGCTG
TTCCTACTTTGACTACAGGGGCCAGGGAACCCTGGTCACCGTCTCCT
CA
1.3
SEQ ID NO. 2583
CAGGTGCAGCTGGTGGAGTCTGGGGGAGGCTTGGTACAGCCTGGCAG
GTCCCTGAGACTCTCCTGTGCAGCCTCTGGATTCACCTTTGCTGATT
ATGCCTTGCACTGGGTCCGGCAAGCTCCAGGGAAGGGCCTGGAGTGG
GTCTCAGGTATTAGTTGGAATAGTGGTAGGAAGGACTATGCGGACAC
TGTGAAGGGCCGATTCACCATCTCCAGAGACAACGCCAAGAAGTCCC
TGTATCTGCAAATGAACAGTCTGAGAGCTGAGGACACGGCCATGTAT
TACTGTGCAAAAGAGAAGGGCCTAGGATTTTGTCGTGGTGGTAGCTG
TTCCTACTTTGACTATAGGGGCCAGGGAACCCTGGTCACCGTCTCCT
CA
1.4
SEQ ID NO. 2584
CAGGTGCAGCTGGTGGAGTCTGGGGGAGGCTTGGTACAGCCTGGCAG
GTCCCTGAGACTCTCCTGTGCAGCCTCTGGATTCACCTTTGCTGATT
ATGCCTTGCACTGGGTCCGGCAAGCTCCAGGGAAGGGCCTGGAGTGG
GTCTCAGGTATTAGTTGGAATGCCGGTAGGAAGGACTATGCGGACAC
TGTGAAGGGCCGATTCACCATCTCCAGAGACAACGCCAAGAAGTCCC
TGTATCTGCAAATGAACAGTCTGAGAGCTGAGGACACGGCCATGTAT
TACTGTGCAAAAGAGAAGGGCCTAGGATTTTGTCGTGGTGGTAGCTG
TTCCTACTTTGACTATAGGGGCCAGGGAACCCTGGTCACCGTCTCCT
CA
1.5
SEQ ID NO. 2585
GAGGTGCAGCTGGTGGAGTCTGGGGGAGGCTTGGTACAGCCTGGCAG
GTCCCTGAGACTCTCCTGTGCAGCCTCTGGATTCACCTTTGCTGATT
ATGCCTTGCACTGGGTCCGACAAGCTCCAGGAAAGGGCCTGGAGTGG
GTCTCAGGTATTAGTTGGAATAGTGGTAGGAAGGACTATGCGGACAC
TGTGAAGGGCCGATTCACCATCTCCAGAGACAACGCCAAGAACTCCC
TGTATCTGCAAATGAACAGTCTGAGAGCTGAGGACACGGCCTTATAT
TACTGTGCAAAGGAGAAGGGCCTAGGATATTGTCGTGGTGGTAGCTG
TTCCTACTTTGACTACAGGGGCCAGGGAACCCTGGTCACCGTCTCCT
CA
1.6
SEQ ID NO. 2586
GAAGTGCAGCTGGTTGAGAGCGGTGGTGGTCTGGTTCAGCCGGGTCG
CAGCCTGCGCCTGAGCTGCGCGGCTAGCGGCTTTACCTTCGCAGATT
ACGCCCTGCATTGGGTTCGTCAAGCACCGGGTAAAGGCCTGGAGTGG
GTGTCCGGCATCTCTTGGAACGCGGGTCGTAAGGATTATGCGGACAC
GGTCAAGGGTCGCTTCACCATTAGCCGTGACAATGCGAAAAATAGCC
TGTATTTGCAGATGAACAGCCTGCGTGCGGAAGATACCGCGCTGTAT
TACTGCGCGAAAGAAAAGGGCTTGGGCTATTGTCGTGGTGGCAGCTG
TTCGTACTTTGACTACCGTGGTCAGGGTACGCTGGTGACGGTCTCGA
GC
2.1
SEQ ID NO. 2587
CAGGTCCAGCTGGTGCAGTCTGGGGCTGAGGTGAAGAAGCCTGGGGC
CTCAGTGAAGGTCTCCTGCAAGGCTTCTGGATACCCCTTCACCAGTT
ATGATATCAATTGGGTGCGACAGGCCACAGGACAAAGCCTTGAGTGG
ATGGGATGGATGAACCCTAACAGTGGTGACACAGTCTATGCACAGAA
ATTCCAGGGCAGAGTCACCATGACCAGGAATACCTCCATAAGCACAG
CCTACATGGAGCTGAGCAGCCTGAGATCTGAGGACACGGCCGTGTAT
TTTTGTGCGAGAGGCAGAAGGGATGACTGGAAGAACAATTATTGGGG
CCAGGGAACCCTGGTCACTGTCTCCTCA
2.2
SEQ ID NO. 2588
CAGGTCCAGCTGGTGCAGTCTGGGGCTGAGGTGAAGAAGCCTGGGGC
CTCAGTGAAGGTCTCCTGCAAGGCTTCTGGATACCCCTTCACCAGTT
ATGATATCAATTGGGTGCGACAGGCCACAGGACGAAGCCTTGAGTGG
ATGGGATGGATGAACCCTACCAATGGTAACACAGTCTATGCACAGAA
ATTCCAGGACAGAGTCACCATGACCAGGAATACCTCCATAAGCACAG
CCTACATGGAGCTGAGCAGCCTGAGATCTGAGGACACGGCCGTGTAT
TTTTGTGCGAGAGGCAGAAGGGATGACTGGAAGAACAATTATTGGGG
CCAGGGAACCCTGGTCACTGTCTCCTCA
2.3
SEQ ID NO. 2589
CAGGTCCAGCTGGTGCAGTCTGGGGCTGAGGTGAAGAAGCCTGGGGC
CTCAGTGAAGGTCTCCTGCAAGGCTTCTGGATACCCCTTCACCAGTT
ATGATATCAATTGGGTGCGACAGGCCACAGGACGAAGCCTTGAGTGG
ATGGGATGGATGAACCCTACAAGTGGTGACACAGTCTATGCACAGAA
ATTCCAGGACAGAGTCACCATGACCAGGAATACCTCCATAAGCACAG
CCTACATGGAGCTGAGCAGCCTGAGATCTGAGGACACGGCCGTGTAT
TTTTGTGCGAGAGGCAGAAGGGATAACTGGAAGAACAATTATTGGGG
CCAGGGAACCCTGGTCACTGTCTCCTCA
3.1
SEQ ID NO. 2590
GAGGTGCAGCTGGTGGAGTCTGGGGGAGGCTTGGTCCAGCCTGGGGG
GTCCCTGAGACTCTCCTGTGCAGCCTCTGGATTTCCCTTTAGTACCT
ATTGGATGAGGTGGCTCCGCCAGGCTCCAGGGAAGGGCCTGGAGTGG
GTGGCCAACATAAACCAAGATGGAAGTGAGAAATACTATGTGGACTC
TGTGAAGGGCCGATTCACCATTTCCAGAGACAACGCCAAGAGTTCAC
TGTTTCTGCAAATGAACAGCCTGAGAGCCGAGGACACGGCTGTATAT
TACTGTGCGAGAGGGTGGGAGTCGGGGTGGTTCGAACCCTGGGGCCA
GGGAACCCTGGTCACCGTCTCCTCA
4.1
SEQ ID NO. 2591
CAGGTTCAGCTGGTGCAGTCTGGGGCTGAGGTGAAGAAGCCTGGGGC
CTCAGTGAAGGTCTCCTGCAAGGCTTCTGGATACCCCTTCACCAATT
ATGATATCAGCTGGATACGACAGGCCACTGGACAAAGTCTTGAGTGG
ATGGGATGGATGAACCCTGACAGTGGTAACACAGGCTATGCACAGAA
GTTCCAGGGCAGAGTCACCATGACCAGGAACACCTCCATAAGCACAG
CCTACATGGAACTGAGTGGCCTGAGATCTGAGGACACGGCCGTATAT
TTCTGTGCGAGAGGGGGTTACAATGCCTGGAGAACGGACTACTGGGG
CCAGGGCACCCTGGTCACCGTCTCCTCA
5.1
SEQ ID NO. 2592
GAGGTGCAGCTGGTGGAGTCTGGGGGAGGCTTGGTCAAGCCTGGAGG
GTCCCTGAGACTCTCCTGTGAAGCCTCTGGATTCACCTTCAGTGACT
TCGACATGAGCTGGATCCGCCAGGCTCCAGGGAAGGGGCTGGAGTGG
GTTGCATACATTAGTAGTAGTGATAGTACCATATATTATAGAGACTC
TGTGAAGGGCCGATTCACCATTTCCAGGGACAACGCCAAGAACTCAC
TGTATCTGCAAATGAACAGCCTGAGAGCCGAAGACACGGCCGTGTAT
TACTGTTCGAGAAACGGGGCCCGGTATAACTGGAACTACGGGGACTT
CCAGCACTGGGGCCAGGGCACCCTGGTCACTGTCTCCTCA
6.1
SEQ ID NO. 2593
GAGGTGCAGCTGGTGGAGTCTGGGGGAGGCTTGGTCAAGCCTGGAGG
GTCCCTGAGACTCTCCTGTGCAGCCTCTGGATTCACCTTCAGTGACG
ACTACATGAGCTGGCTCCGCCAGGCTCCAGGGAAGGGGCTGGAGTGG
GTTTCATACATTAGTAGTAGTGGTAGTACCATATACTACGCAGACTC
TGTGAAGGGCCGATTCACCATCTCCAGGGACAACGCCAAGAACTCAC
TGTATCTGCAAATGAACAGCCTGAGAGCCGAGGACTCGGCCGTGTAT
TACTGTGCGAGAAAAGATATAACGAATATAGCAGTGGGCTCCCTCGG
CTACTGGGGCCAGGGAACCCTGGTCACCGTCTCCTCA
7.1
SEQ ID NO. 2594
GAGGTGCAGCTGGTGGAGTCTGGGGGAGGCTTGGTCCAGCCTGGGGG
GTCCCTGAGACTCTCCTGTGCAGCCTCAGGATTCACCTTTAGTAACT
ATTGGATGAGCTGGGTCCGCCAGGCTCGAGGGAAGGGGCTGGAGTGG
GTGGCCAACATAAGACCAGATGGAAGTGAGCGATACTATGTGGACTC
TGTGAAGGGCCGATTCACCATCTCCAGAGACAACGCCAAGAACTCAT
TGTATCTGCAGATGAGCAGCCTGAGAGCCGAGGACACGGCTGTGTAT
TACTGTGCGAGATCGAGAGATTGGGGATCTCGGGCTTTTGATATCTG
GGGCCAAGGGACAATGGTCACCGTCTCCTCA

Nucleic acid according to the present invention may comprise DNA or RNA and may be wholly or partially synthetic or recombinantly produced. Reference to a nucleotide sequence as set out herein encompasses a DNA molecule with the specified sequence, and encompasses a RNA molecule with the specified sequence in which U is substituted for T, unless context requires otherwise.

Furthermore, the invention relates to a nucleic acid construct comprising at least one nucleic acid defined above. The construct may be in the form of plasmids, vectors, transcription or expression cassettes.

The invention also relates to an isolated recombinant host cell comprising one or more nucleic acid constructs as above.

The invention also relates to a binding agent capable of binding to IL-17RA that competes for binding to IL-17RA with a binding molecule of the invention as described above in a competitive assay.

The invention also relates to an isolated V_{H} domain comprising an amino acid product of or derived from a human V_{H} germline sequence, for example a human V_{H} 3-09, V_{H}1 -08, V_{H} 3-07 or V_{H} 3-11 germline sequence.

The binding molecules of the invention have certain functional properties as described below and set out in the examples.

In particular, the binding molecules of the invention block the effects of IL-17RA on its target cells and are thus indicated for use in the treatment of IL-17RA-mediated diseases and disorders, for example as described herein.

These and other pharmacological activities of the binding molecules of the invention may be demonstrated in standard test methods for example as described in the art, e.g., “Neutralization of IL-17R dependent production of interleukin-6 by primary human fibroblasts: The production of IL-6 in primary human (dermal) fibroblasts is dependent on IL-17” (Hwang S Y et al., (2004) Arthritis Res Ther; 6:R120-128)) and in the examples herein. Thus, as described in more detail in the examples, binding members according to the invention neutralize IL-17RA with high potency. The term “neutralizing” thus refers to neutralization of a biological activity of IL-17R when a binding protein specifically binds IL-17R. Inhibition of a biological activity of IL-17R by a neutralizing binding protein can be assessed by measuring one or more indicators of IL-17R biological activity well known in the art as described in the examples.

For example, neutralisation of IL-17R binding to its receptor may be measured as cellular release of a biological molecule, e.g., MMP13, PGE2 or a cytokine such as IL-6 or IL-8, in a biological assay, since IL-17RA binding to its receptor induces cellular release of these molecules, which can be determined using appropriate assays, e.g. in HT1080 cells, chondrocytes or other suitable cell or tissue types.

Inhibition of biological activity may be partial or total. In specific embodiments, binding members are provided that inhibit IL-17R biological activity by at least 95%, at least 90%, at least 85%, at least 80%, at least 75%, at least 70%, at least 60%, or at least 50% of the activity in absence of the binding member. The degree to which a binding member neutralises IL-17RA is referred to as its neutralising potency. Potency may be determined or measured using one or more assays known to the skilled person and/or as described or referred to herein. For example, potency may be assayed in:
HTRF(R) (Homogeneous Time-Resolved Fluorescence) receptor-ligand binding assay
   HT1080 IL-6 release assay—HT1080 cell assay using synergised IL-6 release in response to IL-17 and TNFα
   Chondrocyte IL-6/IL-8/MMP13/PGE2-release assay IL-6 release assay in cartilage explants
   IL-6 release assay in synovial fibroblasts (e.g. from RA or OA patients), e.g. using synergised IL-6 response to IL-17 and TNFα.

Assays methods are described in detail in the examples.

Neutralising potency of a binding member as calculated in an assay using IL-17 from a first species (e.g. human) may be compared with neutralising potency of the binding member in the same assay using IL-17RA from a second species (e.g., cynomolgus), in order to assess the extent of cross-reactivity of the binding member for IL-17RA of the two species.

Potency is normally expressed as an IC₅₀ value, in nM unless otherwise stated. In functional assays, IC₅₀ is the concentration of a binding member that reduces a biological response by 50% of its maximum. IC₅₀ may be calculated by plotting % of maximal biological response as a function of the log of the binding member concentration, and using a software program to fit a sigmoidal function to the data to generate IC₅₀ values.

In another aspect, the invention thus relates to a binding molecule comprising at least one V_{H} domain directed against human IL-17A, or comprising or consisting of at least one immunoglobulin single V_{H} domain antibody directed against IL-17RA, preferably human IL-17RA, wherein said domain is a human V_{H} domain and has an IC₅₀ for inhibition of IL-6 production of about 0.2 to about 500 nM or more, for example 0.2 to 400, 0.2 to 300, 0.2 to 200, 0.2 to 100, 0.2 to 50, 0.2 to 40, 0.2 to 30, 0.2 to 20, 0.2 to 10, 0.2 to 9, 0.2 to 8, 0.2 to 7, 0.2 to 6, 0.2 to 5, 0.2 to 4.0, 0.2 to, 0.2 to 2 or 0.2 to 1 when tested as described in the examples, i.e. by measuring the ability of IL-17RA binding V_{H} to inhibit IL-17RA induced IL-6 release from the cell line HT1080. The binding molecules of the invention may have an IC₅₀ for inhibition of IL-6 production of less than about 500 nM, preferably less than about 100 nM assessed by measuring the ability of IL-17RA binding V_{H} to inhibit IL-17RA induced IL-6 release from the cell line HT1080. This assay measures IL-6 release, a detailed method is given in the examples. The binding molecule, for example a V_{H} domain, having these binding characteristics may be selected from one of the sequences disclosed herein. In another embodiment, the V_{H} domain comprises a CDR3 sequence or V_{H} sequence as described herein.

In one embodiment, said IL-17RA binding molecule comprises a family 1 or family 1-like V_{H} sequence or part thereof, for example a CDR3 sequence, as described above. In one embodiment, said IL-17RA binding molecule comprises a family 2 or family 2-like V_{H} sequence or part thereof, for example a CDR3 sequence, as described above. In one embodiment, said IL-17RA binding molecule comprises a family 3 or family 3-like V_{H} sequence or part thereof, for example a CDR3 sequence, as described above. In one embodiment, said IL-17RA binding molecule comprises a family 4 or family 4-like V_{H} sequence or part thereof, for example a CDR3 sequence, as described above. In one embodiment, said IL-17RA binding molecule comprises a family 5 or family 5-like V_{H} sequence or part thereof, for example a CDR3 sequence, as described above. In one embodiment, said IL-17RA binding molecule comprises a family 6 or family 6-like V_{H} sequence or part thereof, for example a CDR3 sequence, as described above. In one embodiment, said IL-17RA binding molecule comprises a family 7 or family 7-like V_{H} sequence or part thereof, for example a CDR3 sequence, as described above. Various embodiments of these sequences are detailed above.

Additionally, binding kinetics and affinity (expressed as the equilibrium dissociation constant, KD) of IL-17RA binding molecules of the invention for binding IL-17RA may be determined, e.g. using surface plasmon resonance such as BIAcore®, or KD may be estimated from pA2 analysis.

In another aspect, the invention relates to a binding molecule that has a KD (M) value of in the range of from about 1E-07 (1×10⁻⁷) to about 6E-11 (6×10⁻¹¹), wherein said KD is calculated using BIAcore®. The term “KD” refers to the “equilibrium dissociation constant” and refers to the value obtained in a titration measurement at equilibrium, or by dividing the dissociation rate constant (Koff) by the association rate constant (Kon). In one embodiment, the KD may be as shown in the examples.

In one embodiment, said IL-17RA binding molecule comprises a family 1 or family 1-like V_{H} sequence or part thereof, for example a CDR3 sequence, as described above. In one embodiment, said IL-17RA binding molecule comprises a family 21 or family 2-like V_{H} sequence or part thereof, for example a CDR3 sequence, as described above. In one embodiment, said IL-17RA binding molecule comprises a family 3 or family 3-like V_{H} sequence or part thereof, for example a CDR3 sequence, as described above. In one embodiment, said IL-17RA binding molecule comprises a family 4 or family 4-like V_{H} sequence or part thereof, for example a CDR3 sequence, as described above. In one embodiment, said IL-17RA binding molecule comprises a family 5 or family 5-like V_{H} sequence or part thereof, for example a CDR3 sequence, as described above. In one embodiment, said IL-17RA binding molecule comprises a family 6 or family 6-like V_{H} sequence or part thereof, for example a CDR3 sequence, as described above. In one embodiment, said IL-17RA binding molecule comprises a family 7 or family 7-like V_{H} sequence or part thereof, for example a CDR3 sequence, as described above. Various embodiments of these sequences are detailed above.

In one embodiment, the binding molecule has a KD as defined above and an IC₅₀ for inhibition of IL-6 production as defined above.

A skilled person will know that there are different ways to identify and obtain the antigen binding molecules as described herein, including in vitro and in vivo expression libraries. This is further described in the examples. Optimisation techniques known in the art, such as display techniques (e.g., ribosome display and/or phage display) and/or mutagenesis techniques (e.g., error prone mutagenesis) can be used.

Methods for preparing or generating the polypeptides, nucleic acids, host cells, products and compositions described herein using in vitro expression libraries can comprise the steps of:
a) providing a set, collection or library of nucleic acid sequences encoding amino acid sequences; and
b) screening said set, collection or library of amino acid sequences for amino acid sequences that can bind to/have affinity for IL-17RA and
c) isolating the amino acid sequence(s) that can bind to/have affinity for IL-17RA.

In the above methods, the set, collection or library of sequences may be displayed on a phage, phagemid, ribosome or suitable micro-organism (such as yeast), such as to facilitate screening. Suitable methods, techniques and host organisms for displaying and screening (a set, collection or library of) sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty).

The binding molecules described herein comprising V_{H} domains, can be expressed in a transgenic rodent. The transgenic rodent, for example a mouse, has a reduced capacity to express endogenous antibody genes. Thus, in one embodiment, the rodent has a reduced capacity to express endogenous light and/or heavy chain antibody genes. The rodent may therefore comprise additional modifications to disrupt expression of endogenous light and/or heavy chain antibody genes so that no functional light and/or heavy chains are produced.

In one embodiment, the rodent is a mouse. The mouse may comprise a non-functional lambda light chain locus. Thus, the mouse does not make a functional endogenous lambda light chain. In one embodiment, the lambda light chain locus is deleted in part or completely or rendered non-functional through insertion, inversion, a recombination event, gene editing or gene silencing. For example, at least the constant region genes C1, C2 and C3 may be deleted or rendered non-functional through insertion. In one embodiment, the locus is functionally silenced so that mouse does not make a functional endogenous lambda light chain.

Furthermore, the mouse may comprise a non-functional kappa light chain locus. Thus, the mouse does not make a functional endogenous kappa light chain. In one embodiment, the kappa light chain locus is deleted in part or completely or rendered non-functional through insertion, inversion, a recombination event, gene editing or gene silencing. In one embodiment, the locus is functionally silenced so that the mouse does not make a functional endogenous kappa light chain.

The mouse having functionally silenced endogenous lambda and kappa L-chain loci may, for example, be made as disclosed in WO 2003/000737, which is hereby incorporated by reference in its entirety.

Furthermore, the mouse may comprise a non-functional heavy chain locus. Thus, the mouse does not make a functional endogenous heavy chain. In one embodiment, the heavy chain locus is deleted in part or completely or rendered non-functional through insertion, inversion, a recombination event, gene editing or gene silencing. In one embodiment, the locus is functionally silenced so that the mouse does not make a functional endogenous heavy chain. In one embodiment, the locus is functionally silenced so that mouse does not make a functional endogenous heavy chain.

For example, as described in WO 2004/076618 (hereby incorporated by reference in its entirety), all 8 endogenous heavy chain constant region immunoglobulin genes (μ, δ, γ3, γ1, γ2a, γ2b, ε and α) are absent in the mouse, or partially absent to the extent that they are non-functional, or genes δ, γ3, γ1, γ2a, γ2b and ε are absent and the flanking genes μ and a are partially absent to the extent that they are rendered non-functional, or genes μ, δ, γ3, γ1, γ2a, γ2b and ε are absent and a is partially absent to the extent that it is rendered non-functional, or δ, γ3, γ1, γ2a, γ2b, ε and α are absent and μ is partially absent to the extent that it is rendered non-functional. By deletion in part is meant that the endogenous locus gene sequence has been deleted or disrupted, for example by an insertion, to the extent that no functional endogenous gene product is encoded by the locus, i.e. that no functional product is expressed from the locus. In another embodiment, the locus is functionally silenced.

In one embodiment, the mouse comprises a non-functional endogenous heavy chain locus, a non-functional endogenous lambda light chain locus and a non-functional endogenous kappa light chain locus. The mouse therefore does not produce any functional endogenous light or heavy chains. Thus, the mouse is a triple knockout (TKO) mouse.

The transgenic mouse may comprise a vector, for example a Yeast Artificial Chromosome (YAC) for expressing a heterologous heavy chain locus. YACs are vectors that can be employed for the cloning of very large DNA inserts in yeast. As well as comprising all three cis-acting structural elements essential for behaving like natural yeast chromosomes (an autonomously replicating sequence (ARS), a centromere (CEN) and two telomeres (TEL)), their capacity to accept large DNA inserts enables them to reach the minimum size (150 kb) required for chromosome-like stability and for fidelity of transmission in yeast cells. The construction and use of YACs is well known in the art (e.g. Bruschi, C. V. and Gjuracic, K. Yeast Artificial Chromosomes, ENCYCLOPEDIA OF LIFE SCIENCES 2002 Macmillan Publishers Ltd, Nature Publishing Group/www.els.net).

For example, the YAC may comprise a plethora of human V_{H}, D and J genes in combination with mouse immunoglobulin constant region genes lacking C_{H}1 domains, mouse enhancer and regulatory regions. An example of such a YAC is provided in the example section.

Alternative methods known in the art may be used for deletion or inactivation of endogenous mouse or rat immunoglobulin genes and introduction of human V_{H}, D and J genes in combination with mouse immunoglobulin constant region genes lacking C_{H}1 domains, mouse enhancer and regulatory regions

Transgenic mice can be created according to standard techniques as illustrated in the examples. The two most characterised routes for creating transgenic mice are via pronuclear microinjection of genetic material into freshly fertilised oocytes or via the introduction of stably transfected embryonic stem cells into morula or blastocyst stage embryos. Regardless of how the genetic material is introduced, the manipulated embryos are transferred to pseudo-pregnant female recipients where pregnancy continues and candidate transgenic pups are born.

The main differences between these broad methods are that ES clones can be screened extensively before their use to create a transgenic animal. In contrast, pronuclear microinjection relies on the genetic material integrating to the host genome after its introduction and, generally speaking, the successful incorporation of the transgene cannot be confirmed until after pups are born.

There are many methods known in the art to both assist with and determine whether successful integration of transgenes occurs. Transgenic animals can be generated by multiple means including random integration of the construct into the genome, site-specific integration, or homologous recombination. There are various tools and techniques that can be used to both drive and select for transgene integration and subsequent modification including the use of drug resistance markers (positive selection), recombinases, recombination-mediated cassette exchange, negative selection techniques, and nucleases to improve the efficiency of recombination. Most of these methods are commonly used in the modification of ES cells. However, some of the techniques may have utility for enhancing transgenesis mediated via pronuclear injection.

Further refinements can be used to give more efficient generation of the transgenic line within the desired background. As described above, in preferred embodiments, the endogenous mouse immunoglobulin expression is silenced to permit sole use of the introduced transgene for the expression of the heavy-chain only repertoire that can be exploited for drug discovery. Genetically-manipulated mice, for example TKO mice that are silenced for all endogenous immunoglobulin loci (mouse heavy chain, mouse kappa chain and mouse lambda chain) can be used as described above. The transfer of any introduced transgene to this TKO background can be achieved via breeding, (either conventional or with the inclusion of an IVF step to give efficient scaling of the process). However, it is also possible to include the TKO background during the transgenesis procedure. For example, for microinjection, the oocytes may be derived from TKO donors. Similarly, ES cells from TKO embryos can be derived for use in transgenesis.

The invention also relates to a method for producing a binding molecule comprising at least one immunoglobulin single domain antibody directed against IL-17RA wherein said domain is a human V_{H} domain said method comprising
a) immunising a transgenic mouse that expresses a nucleic acid construct comprising human heavy chain V genes and that is not capable of making functional endogenous light or heavy chains with an IL-17RA antigen,
b) generating a library from said mouse and
c) isolating V_{H} domains from said libraries.

Additional steps to optimize the sequences can be included.

The invention also relates to a binding molecule capable of binding human IL-17RA comprising a V_{H} domain obtained or obtainable from a mouse that is not capable of making functional endogenous light or heavy chains, for example through the method described above.

The binding molecule of the invention may be conjugated to another moiety. This can be selected from a toxin, enzyme, radioisotope, other detectable label, peptide, protein and chemical moiety of interest.

For example, the binding molecule of the invention may be labelled with a detectable or functional label. A label can be any molecule that produces or can be induced to produce a signal, including but not limited to fluorescers, radiolabels, enzymes, chemiluminescers or photosensitizers. Thus, binding may be detected and/or measured by detecting fluorescence or luminescence, radioactivity, enzyme activity or light absorbance.

Half-life of the binding molecule of the invention can be increased by a chemical modification, especially by PEGylation, or by incorporation in a liposome or linking to another molecule, e.g. serum albumin or an anti-HSA binding molecule.

In one embodiment, a binding molecule of the invention is covalently modified. The term “covalently modified/covalent modification” includes modifications of a binding molecule according to the present invention, e.g. of a specified sequence; with an organic proteinaceous or non-proteinaceous derivatizing agent, fusions to heterologous polypeptide sequences, and post-translational modifications. Covalent modified polypeptides, e.g., of a specified sequence, still have the functional properties described herein, for example the ability to bind the human IL-17 or e.g. neutralize IL-6 production of IL-17 induced human dermal fibroblasts by crosslinking. Covalent modifications are generally introduced by reacting targeted amino acid residues with an organic derivatizing agent that is capable of reacting with selected sides or terminal residues, or by harnessing mechanisms of post-translational modifications that function in selected recombinant host cells. Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deaminated under mildly acidic conditions. Other post-translational modifications include hydroxylation of proline and lysine, phosphorylation of hydroxyl groups of seryl, tyrosine or threonyl residues, methylation of the [alpha]-amino groups of lysine, arginine, and histidine side chains. Covalent modifications e.g. include fusion proteins comprising a binding molecule according to the present invention, e.g. of a specified sequence and their amino acid sequence variants, such as immunoadhesins, and N-terminal fusions to heterologous signal sequences.

In another aspect of the present invention, there is provided a pharmaceutical composition comprising an IL-17RA binding molecule according to the present invention and a pharmaceutically acceptable carrier. The binding molecule of the present invention or a composition thereof can be administered by any convenient route and examples of the administration form of the binding molecule or composition of the present invention include without limitation topical, in particular dermal, parenteral, and intranasal. Parenteral administration includes subcutaneous injections, intravenous, intramuscular, intrasternal injection or infusion techniques. Compositions can take the form of one or more dosage units.

The composition of the invention can be in the form of a liquid, e. g. a solution, emulsion or suspension. The liquid can be useful for delivery by dermal, topical or injection routes. The liquid compositions of the invention, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A composition can be enclosed in an ampoule, a disposable syringe or a multiple-dose vial made of glass, plastic or other material.

In specific embodiments, it can be desirable to administer a binding molecule of the present invention or composition thereof locally to the area in need of treatment.

Thus, in a preferred embodiment of all aspects of the invention, administration of the composition or binding molecule of the invention is by topical administration to healthy or diseased skin. The binding molecule is capable of penetrating at least the outer layer of the skin and can therefore be delivered dermally or transdermally. Accordingly, in one embodiment of the various aspects of the invention, topical delivery of the the composition or binding molecule of the invention to the skin is direct delivery into the skin for local non-systemic exposure. In another embodiment, topical delivery of the composition or binding molecule of the invention to the skin is direct delivery to the skin to provide systemic exposure as the _{H} domain penetrates through all layers of the skin.

The skin that is treated may be diseased or healthy skin. In a preferred embodiment, the skin disease is psoriasis or atopic dermatitis.

Preferably, the surface area to which it is applied is 1%-30% of the body surface area, for example 1%-10% or 1-20%. Administration may thus be to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% of body surface area. In one embodiment, the disease state is mild. In another embodiment, the disease state is moderate. In another embodiment, the disease state is severe. For the treatment of psoriasis, administration is to areas affected, typically one or more affected area selected from elbows, knees, palms of hands, scalp, soles of feet, genitals, upper thighs, groin, buttocks, face and torso. For the treatment of atopic dermatitis administration is to areas affected, typically one or more affected area selected from face, forearms and wrists.

Thus, the binding molecule can be applied directly to diseased or healthy skin in the form of cream, lotion, sprays, solution, gel, ointment, paste, plaster, patch, bioadhesive, suspension or the like, and/or may be prepared so as to contain liposomes, micelles, and/or microspheres. In one embodiment, the binding molecule is applied directly to diseased skin in the form of a liquid (e.g. a spray), plaster, patch or bioadhesive. In one embodiment, the binding molecule is applied directly to diseased skin in the form of a microemulsion.

Microemulsions are generally defined as having a droplet diameter within the range of 2-500 nm thus allowing effective delivery of actives into the skin. Microemulsions have been proposed for use in enhancing transdermal delivery of a range of compounds. This is described in US2007/0243132 incorporated herein in its entirety.

Specifically, as used herein, the term microemulsion refers to a formulation that comprises an oil phase, a water phase and a surfactant, wherein the microemulsion is suitable for transdermal delivery of a binding molecule, for example comprising a human V_{H} domain as described herein. Preferably, the microemulsion of the invention has a droplet diameter within the range of 2-500 nm. In one embodiment, a microemulsion may further comprise a co-surfactant, a co-solvent, or a combination thereof.

The microemulsion of the present invention may be oil-in-water microemulsion, wherein the surfactant is preferentially soluble in water; water-in-oil microemulsion, wherein the surfactant is mainly in the oil phase; a three-phase microemulsion wherein a surfactant-rich middle phase coexists with water and oil phases; a bicontinuous monophase; a single phase micellar solution that forms upon addition of a sufficient quantity of amphiphile (surfactant plus alcohol); or a swollen micellar solution.

The microemulsion of the present invention may be produced by methods known in the art. In general, microemulsions are produced by emulsifying components under conditions including typically sufficient force or the required temperature to generate the required dispersion level, conductivity, viscosity, percolativity or other dispersion characteristics.

Microemulsion formation can be assessed using scattering and spectroscopic techniques such as neutron scattering, time-average scattering, quasi-electric light scattering i.e., high-resolution ultrasonic spectroscopy or photon correlation spectroscopy. The partition coefficients of microemulsions may also be measured chromatographically. The selection of particular formulations is based on a number of different paradigms depending upon the desired application. Illustrative paradigms include the hydrophilic-lipophilic balance, the phase-inversion temperature, or the cohesive-energy ratio. Microemulsions may be formulated using a wide range of immiscible liquids and other additional agents.

A microemulsion of the present invention may comprise an oil phase in the range of from 50 and 99% by weight, most preferably from 50 and 90% by weight; a water phase in the range of from 2 to 50% by weight, most preferably between 1 and 50% by weight; and surfactant in the range of from 0.1 to 90% by weight, preferably in the range of from 1 to 90% by weight surfactant. The microemulsion may further comprise from 0.1 to 90% by weight cosurfactant or cosolvent; preferably from 1 to 90% by weight cosurfactant or cosolvent.

The oil phase may comprise natural oils derived from plants or animals, such as vegetable oils, sunflower oils, coconut oils, almond oils; purified synthetic or natural di or triglycerides (such as Crodamol GTC® and Capmul MC®); phospholipids and their derivatives (such as lecithin or lysolecithin); fatty acid esters (such as isopropyl myristate, isopropyl palmitate, ethyl oleate, oleic acid ethyl ester); hydrocarbons (such as hexane, the n-decane through n-octadecane series); and/or glycerolysed fats and oils (such as glyceryl monooleate, glyceryl monocaprylate, glycerol monocaprate, propylene glycol monocaprylate, propyleme glycol monolaurate).

Other oil phase ingredients include, but are not limited to, Labrafil M 1944 CS™, benzene, tetrahydrofuran, and n-methyl pyrrolidone, or halogenated hydrocarbons, such as methylene chloride, or chloroform. In a particular embodiment, the oil phase comprises Crodamol GTCC® and Capmul MCM®, at 3:1 ratio. The oil component is either used alone or in combination with another oil component or components. Each oil or unique mixture of oils may require a different surfactant or mixture of surfactants or surfactants and co-surfactants to form a microemulsion with the water phase, as can routinely be determined by those of skill in the art. Water phase ingredients may comprise water and any water-soluble components in water, including one or more pharmaceutical agent.

The microemulsion of the present invention may further comprise solvents or other agents to enhance emulsion formation or stability. Other agents may be introduced to provide functions such as pH, ionic content, polymerisation, taste, smell, sterility, colour, viscosity, etc.

The microemulsions of the present invention may also be generated using any suitable synthetic plastic or polymeric, monomeric or hybrid colloidal material.

According to the methods and uses set out above, the binding molecule can be administered together with one or more chemical skin penetration enhancer. Examples of skin penetration enhancers are set out below.

In another embodiment, the binding molecule is administered using occlusion. In one embodiment, the binding molecule is administered to healthy or diseased skin together with a chemical skin penetration enhancer and using occlusion. In one embodiment, the binding molecule is administered to healthy or diseased skin as a microemulsion and using occlusion.

In another embodiment of the various aspects of the invention, administration may be improved using non-chemical skin penetration enhancers, for example phonophoresis, sonophoresis, electroporation or using the microneedle technique. This uses small needles (10-200 μm height and 10-50 μm width) which are connected with the drug reservoir. The microneedle delivery device is applied to the skin surface without reaching the nerve endings of the upper dermis.

The binding molecule administered as set out above is capable of penetrating at least the outer layer of the skin and thus delivers an effective therapeutic amount of the binding molecule to treat the disease. The binding molecule administered as set out herein penetrates the skin in preferably 6 hours or less, for example 1 hour or less.

In one aspect, the invention relates to a pharmaceutical composition comprising a binding molecule of the invention and a skin penetration enhancer that facilitates or improves skin penetration. Unless otherwise specified, the term skin penetration enhancer as used herein refers to a chemical skin penetration enhancer. Numerous chemical penetration enhancers are known in the art and can be used in the composition of the invention. These include, but are not limited to: water, alcohols, preferably alcohols with up to six carbon atoms, for example ethanol, glycols, for example alcohol diethylene glycol (Transcutol®), alkyl-N,N-disubstituted aminoacetates, for example dodecyl-N,N-dimethyl-aminoacetate, esters, for example ethylacetate, Azone® and derivatives, surfactants, for example sodium dodecyl sulphate, terpenes and terpenoids, for example d-Limonene, fatty acids, for example oleic acid, urea and derivatives, for example 1,3-Diphenyl-urea, pyrrolidones, for example N-Methyl-2-pyrrolidone, and 2-pyrrolidone-5-carboxylic acid, cyclodextrins, for example beta-cyclodextrin, sulphoxides, for example dimethylsulphoxide. Other skin penetration enhancers are known to the skilled person. In one embodiment, the enhancer is not water. In one embodiment, the skin penetration enhancers are selected from one or more of Propylene Glycol, Isopropyl Myristate and Azone. Preferred penetration enhancers are DMSO, azone, Transcuto®, isopropyl myristate, oleic acid or combinations thereof, for example as set out in table 6 and in the examples.

In one embodiment, the penetration enhancer is not one or more of water, ethanol, polyethylene glycol derivatives, polyoxyethylene derivatives such as polysorbate, a fatty alcohol such as cetyl alcohol, stearyl alcohol, or cerostearyl alcohol, glycerol and propylene glycol.

The amount of the binding molecule of the present invention that is effective/active in the treatment of a particular disease will depend on the nature of the disease, and can be determined by standard clinical techniques. In addition, in vitro or in vivo assays can optionally be employed to help identify optimal dosage ranges. The precise dose to be employed in the compositions will also depend on the route of administration, and the seriousness of the disease, and should be decided according to the judgment of the practitioner and each patient's circumstances.

Compositions of the invention comprise an effective amount of a binding molecule of the present invention such that a prophylactically- or therapeutically-effective dosage will be obtained. The correct dosage of the compounds will vary according to the particular formulation, the mode of application, and its particular site, and the disease being treated. Other factors like age, body weight, sex, diet, time of administration, rate of excretion, drug combinations, reaction sensitivities and severity of the disease shall be taken into account. Administration can be carried out continuously or periodically within the maximum tolerated dose.

Typically, this amount is at least about 0.01% of a binding molecule of the present invention by weight of the composition.

Preferred compositions of the present invention are prepared so that a parenteral dosage unit contains from about 0.01% to about 2% by weight of the binding molecule of the present invention.

For intravenous administration, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight.

The present compositions can take the form of suitable carriers, such aerosols, sprays, suspensions, or any other form suitable for use. Other examples of suitable pharmaceutical carriers are described in “Remington's Pharmaceutical Sciences” by E. W. Martin.

Liposomes and micelles can also be used according to the invention.

Liposomes are microscopic vesicles having a lipid wall comprising a lipid bilayer, and, in the present context, encapsulate heavy chain only antibody or composition of the invention. Liposomal preparations herein include cationic (positively charged), anionic (negatively charged), and neutral preparations. Cationic liposomes are readily available. For example, N[1-2,3-dioleyloxy)propyl]-N,N,N-triethyl-ammonium (DOTMA) liposomes are available under the tradename Lipofectin® (GIBCO BRL, Grand Island, N.Y.). Similarly, anionic and neutral liposomes are readily available as well or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl-8 dodecyl ether, polyoxyl-12 dodecyl ether, nonoxynol 10, and nonoxynol 30.

Microspheres, similarly, may be incorporated into the present formulations. Like liposomes and micelles, microspheres essentially encapsulate one or more components of the present formulations. They are generally although not necessarily formed from lipids, preferably charged lipids such as phospholipids. Preparation of lipidic microspheres is well known in the art and described in the pertinent texts and literature.

The pharmaceutical compositions can be prepared using methodology well known in the pharmaceutical art. For example, a composition can be prepared by combining a binding molecule of the present invention with water so as to form a solution. A surfactant can be added to facilitate the formation of a homogeneous solution or suspension.

The invention furthermore relates to a method for the prevention and/or treatment of a disease, said method comprising administering, to a subject in need thereof, a pharmaceutically active amount of a binding molecule of the invention, and/or of a pharmaceutical composition of the invention. More in particular, the invention relates to a method for the prevention and/or treatment of a disease selected from the non-limiting group consisting of the diseases listed herein, said method comprising administering, to a subject in need thereof, a pharmaceutically active amount of a binding molecule of the invention, and/or of a pharmaceutical composition of the invention. Examples of the immune related diseases that can be treated according to the invention will be clear to the skilled person based on the disclosure herein, and for example include autoimmune diseases, inflammatory conditions, allergies and allergic conditions, hypersensitivity reactions, severe infections, and organ or tissue transplant rejection.

The invention also relates to a binding molecule or pharmaceutical composition of the invention for use in the treatment of disease. In another aspect, the invention relates to a binding molecule of the invention for use in the treatment of a disease, for example autoimmune diseases, inflammatory conditions, allergies and allergic conditions, hypersensitivity reactions, severe infections, and organ or tissue transplant rejection.

In another aspect, the invention relates to the use of a binding molecule of the invention in the manufacture of a medicament for the treatment of a disease, for example autoimmune diseases, inflammatory conditions, allergies and allergic conditions, hypersensitivity reactions, severe infections, and organ or tissue transplant rejection.

The disease according to the aspects set out above may be selected from the following non-limiting list: psoriasis, systemic lupus erythematosis, rheumatoid arthritis, osteoarthritis, juvenile chronic arthritis, spondyloarthropathies, systemic sclerosis, idiopathic inflammatory myopathies, Sjogren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain Barre syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, autoimmune haematological disorders (including e.g. hemolytic anaemia, aplastic anaemia, pure red cell anaemia and idiopathic thrombocytopenia), autoimmune inflammatory bowel disease (including e.g. ulcerative colitis, Crohn's disease and Irritable Bowel Syndrome), transplantation associated diseases including graft rejection and graft-versus-host-disease.

In a preferred embodiment, the disease is selected from psoriasis, spondyloarthropathies, uveitis and atopic dermatitis. In another embodiment, the disease is asthma.

Binding molecules of the invention are also useful for the treatment, prevention, or amelioration of asthma, bronchitis, pneumoconiosis, pulmonary emphysema, and other obstructive or inflammatory diseases of the airways. Binding molecules of the invention are useful for treating undesirable acute and hyperacute inflammatory reactions which are mediated by IL-17RA, or involve IL-17RA production, or the promotion of TNF release by IL-17RA, e.g. acute infections, for example septic shock (e.g., endotoxic shock and adult respiratory distress syndrome), meningitis, pneumonia; and severe burns; and for the treatment of cachexia or wasting syndrome associated with morbid TNF release, consequent to infection, cancer, or organ dysfunction, especially AIDS-related cachexia, e.g., associated with or consequential to HTV infection.

Binding molecules of the invention are particularly useful for treating diseases of bone metabolism including osteoarthritis, osteoporosis and other inflammatory arthritis, and bone loss in general, including age-related bone loss, and in particular periodontal disease.

Binding molecules of the invention may be administered as the sole active ingredient or in combination with one or more other drug, e.g., immunosuppressive or immunomodulating agents or other anti-inflammatory agents, e.g., for the treatment or prevention of diseases mentioned above. For example, the binding molecule of the invention maybe used in combination with immunosuppressive monoclonal antibodies, e.g., monoclonal antibodies to leukocyte receptors, e.g., MHC, CD2, CD3, CD4, CD7, CD8, CD25, CD28, CD40. CD45, CD58, CD80, CD86 or their ligands; other immunomodulatory compounds, e.g. a recombinant binding molecule having at least a portion of the extracellular domain of CTLA4 or a mutant thereof, e.g., an at least extracellular portion of CTLA4 or a mutant thereof joined to a non-CTLA4 protein sequence, e.g. CTLA4Ig (e.g., designated ATCC 68629) or a mutant thereof, e.g., LEA29Y; adhesion molecule inhibitors, e.g., LFA-I antagonists, ICAM-I or -3 antagonists, VCAM-4 antagonists or VLA-4 antagonists; or a chemotherapeutic agent, e.g., paclitaxel, gemcitabine, cisplatinum, doxorubicin or 5-fluorouracil; anti-TNF agents, e.g., monoclonal antibodies to TNF, e.g., infliximab, adalimumab, CDP870, or receptor constructs to TNF-RI or TNF-RII, e.g., Etanercept™, PEG-TNF-RI; blockers of proinflammatory cytokines, IL-I blockers, e.g., Anakinra™ or IL-1 trap, AAL160, ACZ 885, IL-6 blockers; chemokines blockers, e.g., inhibitors or activators of proteases, e.g. metalloproteases, anti-IL-15 antibodies, anti-IL-6 antibodies, anti-CD20 antibodies, NSAIDs, such as aspirin or an anti-infectious agent. This list is not limited to the agents mentioned.

The binding molecule of pharmaceutical composition of the invention may administered at the same time or at a different time as the other drug. Administration may be simultaneously, sequentially or separately.

The invention also relates to methods for diagnosing a disease. Exemplary diseases are listed above. In one embodiment, the disease is psoriasis. The method comprises determining the level of IL-17RA expression by detecting binding of a binding molecule described herein in a sample and comparing the level of expression of IL-17RA in the test sample with the level of expression in a control sample from a non-psoriatic subject or with a standard value or standard value range for a non-psoriatic subject. An elevation in IL-17RA expression in the test sample relative to the control or standard indicates presence of the disease.

In another aspect, the invention provides a kit comprising a binding molecule of the invention useful for the treatment of a disease described above and optionally instructions for use.

The invention also relates to detection methods using the binding molecule of the invention. Given their ability to bind to human IL-17RA, the human-IL-17RA-binding molecules disclosed herein can be used to detect IL-17RA (e.g., in a biological sample, such as serum or plasma), using a conventional immunoassay, such as an enzyme linked immunosorbent assays (ELISA), a radioimmunoassay (RIA) or tissue immunohistochemistry. A method for detecting IL-17RA in a biological sample is provided comprising contacting a biological sample with a binding molecule disclosed herein and detecting either the binding molecule bound to IL-17RA or unbound binding molecule, to thereby detect IL-17RA in the biological sample. The binding molecule can be directly or indirectly labeled with a detectable substance to facilitate detection of the bound or unbound molecule. Suitable detectable substances include various enzymes, prosthetic groups, fluorescent materials, luminescent materials and radioactive materials.

Alternative to labeling the binding molecule, human IL-17RA can be assayed in biological fluids by a competition immunoassay utilizing IL-17RA standards labeled with a detectable substance and an unlabeled human IL-17RA binding molecule. In this assay, the biological sample, the labeled IL-17RA standards and the human IL-17RA binding molecule are combined and the amount of labeled IL-17RA standard bound to the unlabeled binding molecule is determined. The amount of human IL-17RA in the biological sample is inversely proportional to the amount of labeled IL-17RA standard bound to the IL-17RA binding molecule. Similarly, human IL-17RA can also be assayed in biological fluids by a competition immunoassay utilizing IL-17RA standards labeled with a detectable substance and an unlabeled human IL-17RA binding molecule.

As explained herein, binding molecules of the invention are capable of neutralizing IL-17RA activity, e.g., human IL-17RA activity, both in vitro and in vivo.

Accordingly, such binding molecules disclosed herein can be used to inhibit IL-17RA activity, e.g., in a cell culture containing IL-17RA, in human subjects or in other mammalian subjects having IL-17RA with which a binding molecule disclosed herein cross-reacts. In one embodiment, a method for inhibiting or increasing IL-17RA activity is provided comprising contacting IL-17RA with a binding molecule disclosed herein such that IL-17RA activity is inhibited or increased. For example, in a sample containing, or suspected of containing IL-17RA, a binding molecule disclosed herein can be added to the culture medium to inhibit IL-17RA activity in the sample.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including references to gene accession numbers.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the non-limiting examples.

### EXAMPLES

### Example 1. Construction of Tg/TKO Mice

Mice carrying a heavy-chain antibody transgenic locus in germline configuration within a background that is silenced for endogenous heavy and light chain antibody expression (triple knock-out, or TKO) were created as previously described (WO2004/076618 and WO2003/000737, Ren et al. Genomics, 84, 686, 2004; Zou et al., J. Immunol., 170, 1354, 2003). Briefly, transgenic mice were derived following pronuclear microinjection of freshly fertilised oocytes with a yeast artificial chromosome (YAC) comprising a plethora of human V_{H}, D and J genes in combination with mouse immunoglobulin constant region genes lacking C_{H}1 domains, mouse enhancer and regulatory regions. Yeast artificial chromosomes (YACs) are vectors that can be employed for the cloning of very large DNA inserts in yeast. As well as comprising all three cis-acting structural elements essential for behaving like natural yeast chromosomes (an autonomously replicating sequence (ARS), a centromere (CEN) and two telomeres (TEL)), their capacity to accept large DNA inserts enables them to reach the minimum size (150 kb) required for chromosome-like stability and for fidelity of transmission in yeast cells. The construction and use of YACs is well known in the art (e.g., Bruschi, C. V. and Gjuracic, K. Yeast Artificial Chromosomes, ENCYCLOPEDIA OF LIFE SCIENCES 2002 Macmillan Publishers Ltd, Nature Publishing Group/www.els.net).

The YAC used was about 340 kb comprises 10 human heavy chain V genes in their natural configuration, human heavy chain D and J genes, a murine Cγ1 gene and a murine 3′ enhancer gene. It lacks the C_{H}1 exon. Specifically, the YAC comprised (from 5′ to 3′): telomere-yeast TRP1 marker gene-Centromere-10 human V genes-human D genes-human J genes-mouse p enhancer and switch-mouse Cγ1 (C_{H}1Δ) gene-mouse 3′ enhancer-Hygromycin resistant gene-yeast marker gene HIS3-telomere.

The transgenic founder mice were back-crossed with animals that lacked endogenous immunoglobulin expression to create the Tg/TKO lines used in the immunisation studies described.

### Example 2. Antigen for Immunisation

The immunisations used recombinant purified protein. Recombinant human IL-17RA was purchased from R&D systems (177-IR-100).

### Example 3. Immunisation Protocol

In the present case, recombinant protein was administered to the Tg/TKO. Briefly, mice aged 8-12 weeks of age each received a total of 10 ug of recombinant protein, emulsified in Complete Freund's Adjuvant and delivered subcutaneously, followed by boosts of 1-10 ug of recombinant protein, emulsified in Incomplete Freund's Adjuvant, also administered subcutaneously, given at various intervals following the initial priming. A final dose of antigen was administered intraperitoneally, in phosphate buffered saline, in the absence of adjuvant.

Alternative immunisation routes and procedures can also be employed. For example, different adjuvants or immune potentiating procedures may be used instead of Freund's adjuvant. DNA immunisations are often delivered intramuscularly or via a Genegun. Transfected cells or membrane preparations from such cells are often, although not exclusively, administered intraperitoneally.

### Example 4. Serum ELISA

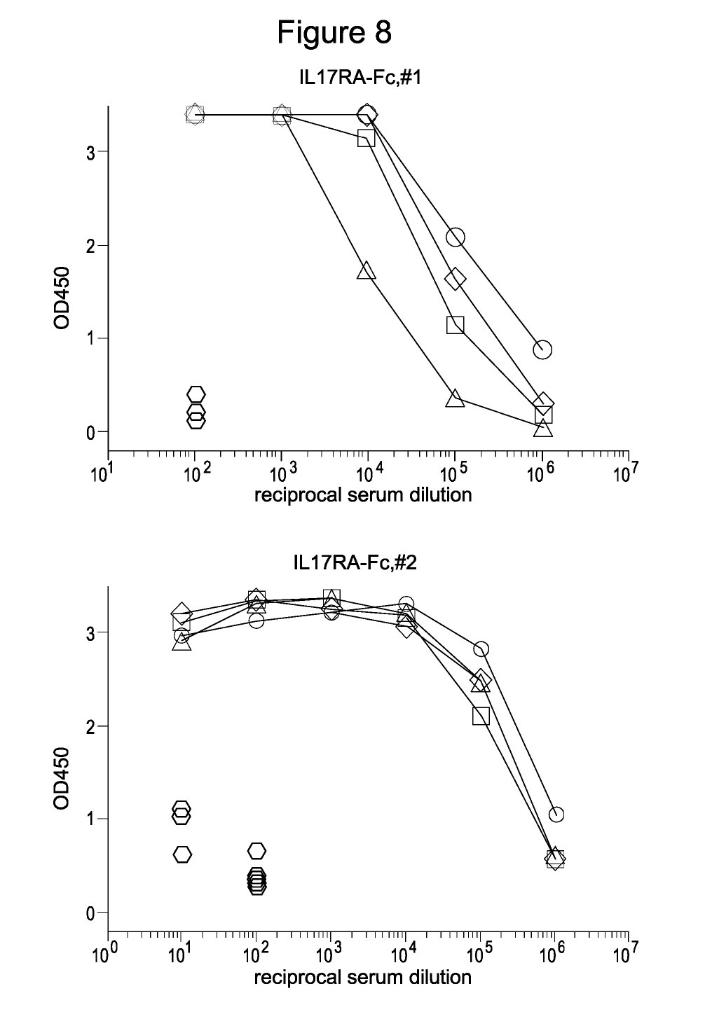

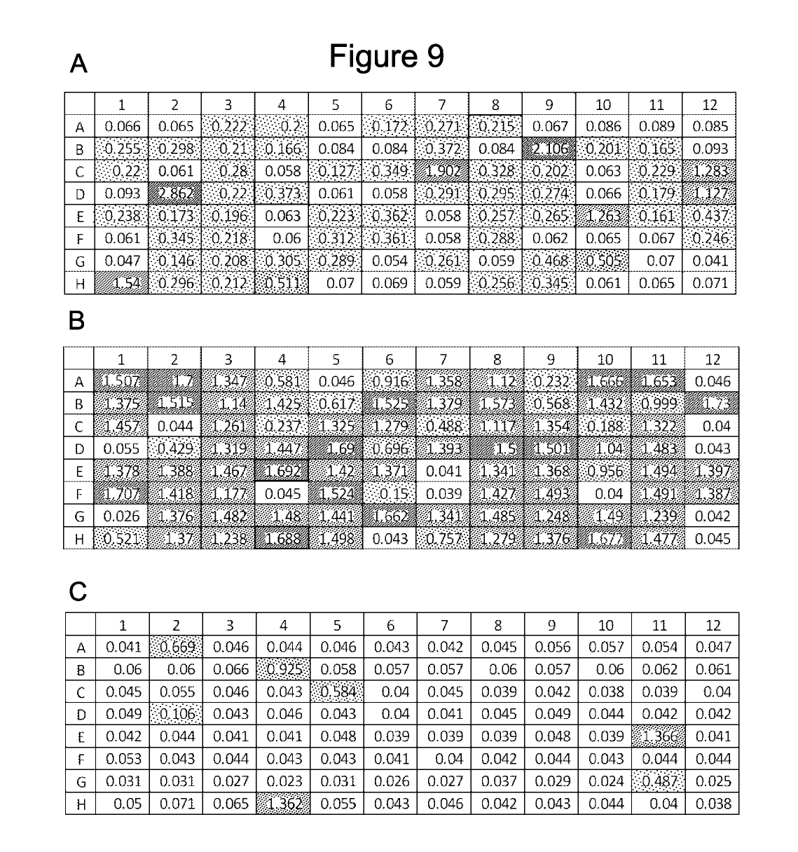

During and following immunisation, serum was collected from mice and checked for the presence of heavy-chain antibody responses to the immunogen by ELISA. Nunc Maxisorp plates (Nunc Cat. No. 443404) were coated overnight at 4° C. with 50 ul/well of a 5 ug recombinant antigen/ml of PBS solution. Following decanting of the antigen solution, plates were washed using PBS (prepared from PBS tablets, Oxoid cat no. BR0014G) supplemented with 0.05% Tween™20 (sigma P1379), followed by washes with PBS without added Tween™. To block non-specific protein interactions, a solution of 3% skimmed milk powder (Marvel) in PBS was added to the wells and the plate was incubated for at least one hour at room temperature. Dilutions of serum in 3% Marvel/PBS were prepared in polypropylene tubes or plates and incubated for at least one hour at room temperature prior to transfer to the blocked ELISA plate where a further incubation of at least one hour took place. Unbound protein was then washed away using repetitive washes with PBS/Tween® followed by PBS. A solution of biotin-conjugated, goat anti mouse IgG, Fcgamma subclass 1 specific antibody (Jackson 115-065-205), prepared in PBS/3% Marvel was then added to each well and a further incubation at room temperature for at least one hour took place. Unbound detection antibody was removed by repeated washing using PBS/Tween® and PBS. Neutravidin-HRP solution (Pierce 31030) in 3% Marvel/PBS was then added to the ELISA plates and allowed to bind for at least 30 minutes. Following further washing, the ELISA was developed using TMB substrate (Sigma cat. no. T0440) and the reaction was stopped after 10 minutes by the addition of 0.5M H₂SO₄ solution (Sigma cat. no. 320501). Absorbances were determined by reading at 450 nm. Examples of Serum ELISA data are shown in FIG. 8. Alternative assays, such as ELISPOT assays, may also be used to check for immunisation induced heavy-chain antibody responses.

### Example 5. Generation of Libraries from Immunised Mice

a. Processing Tissues, RNA Extraction and cDNA Manufacture

Spleen, inguinal and brachial lymph nodes were collected into RNAlater® from each immunised animal. For each animal, ⅓ of the spleen and 4 lymph nodes were processed separately. Initially, the tissues were homogenised; following transfer of tissues to Lysing matrix D bead tubes (MP Bio cat#116913100), 600 ul of RLT buffer containing β-mercaptoethanol (from Qiagen RNeasy® kit cat#74104) was added before homogenisation in a MP Bio Fastprep homogeniser (cat #116004500) using 6 m/s 40 seconds cycles. The tubes containing the homogenised tissues were transferred to ice and debris was pelleted by microcentrifugation at 10 g for 5 minutes. 400 ul of the supernatant was removed and used for RT-PCR.

Initially, RNA was extracted using Qiagen RNeasy® kit cat#74104 following the manufacturer's protocol. Each RNA sample was then used to make cDNA using Superscript III RT-PCR high-fidelity kit (Invitrogen cat #12574-035). For each spleen and LN RNA sample, 5 RT-PCR reactions were performed, each with VH_J/F (long) primer in combination with a primer for V_{H}1, V_{H}2, V_{H}3, V_{H}4 or V_{H}6 family. Details of the primers are below in Table 8.

TABLE 8
Primers

| | |
|---|---|
| V1a/pelB | GCCGCTGGATTGTTATTACTCGCGGCCCAGCCGG |
| (long) | CCATGGCCCAGGTBCAGCTGGTGCAGTCTGGGGC |
| | TGAGG SEQ ID NO 2595 |
| V2/pelB | GCCGCTGGATTGTTATTACTCGCGGCCCAGCCGG |
| (long) | CCATGGCCCAGATCACCTTGAAGGAGTCTGG |
| | SEQ ID NO 2596 |
| V3/pelB | GCCGCTGGATTGTTATTACTCGCGGCCCAGCCGG |
| (long) | CCATGGCCSAGGTGCAGCTGGTGGAGTCTGGGGG |
| | AGG SEQ ID NO 2597 |
| V4- | GCCGCTGGATTGTTATTACTCGCGGCCCAGCCGG |
| 4/pelB | CCATGGCCCAGGTGCAGCTGCAGGAGTCGGG |
| (long) | SEQ ID NO 2598 |
| V6/pelB | GCCGCTGGATTGTTATTACTCGCGGCCCAGCCGG |
| (long) | CCATGGCCCAGGTACAGCTGCAGCAGTCAGG |
| | SEQ ID NO 2599 |
| VH_J/F | CCGTGGTGATGGTGGTGATGGCTACCGCCACCCT |
| (long) | CGAGTGARGAGACRGTGACC |
| | SEQ ID NO 2600 |

Residues in bold have homology with pUCG3

Mastermixes were prepared for the RT-PCR reactions, based on the following tube reaction components.

12.5 μl 2x reaction mix
0.5 μl forward primer (10 uM)
0.5 μl reverse primer (10 uM)
0.5 μl enzyme mix
500 ng-1 μg RNA
Up to 25 μl with water

The RT-PCR reactions were carried out in a thermal cycler using the following conditions;

| | |
|---|---|
| 50° C. 20 min | |
| 94° C. 2 min | |
| 35 cycles of | 94° C. 15 sec |
| | 58° C. 30 sec |
| | 68° C. 30 sec |
| 68° C. 5 min | |
| Hold at 4° C. | |

Products in the range of 370 bp were confirmed by gel electrophoresis.

For each mouse, the V_{H} products amplified for a given family from the ⅓ spleen and each of the 4 lymph nodes were then pooled for purification using Thermo/Fermentas GeneJet PCR purification kit (cat #K0702) which was used according to the Manufacturer's instructions, with the products eluted in 50 ul of water.

b. Cloning into Phagemid Vector

The phagemid vector, pUCG3, was employed in these studies. As indicated, V_{H} may be cloned into pUCG3, using conventional methods involving restriction enzyme digestions with NcoI and XhoI, ligation and transformation. Alternatively, a PCR-based method may be used to construct the V_{H} phagemid libraries. Both of these procedures were used to generate libraries from the amplified V_{H} sequences. The former method is widely used in the art. For the PCR-based method, the following procedure was used:

A linearised version of pUCG3 was created using PCR; with the following primers:

pUCG3-F3
SEQ ID NO. 2601
CTCGAGGGTGGCGGTAGCCATCACCACCATC
pUCG3-R3
SEQ ID NO. 2602
TCCATGGCCATCGCCGGCTGGGCCGCGAG

Phusion High fidelity PCR master mix with GC buffer (cat # F532L, NEB) was used for the PCR reactions which comprised the following reagents;

| Phusion GC 2x mix | 25 | ul |
|---|---|---|
| pUCG3 | 5-10 | ng |
| Primers (10 uM) | 1.25 μl of each | |
| DMSO | 1.5 | μl |

| | |
|---|---|
| Nuclease-free H₂O | to final volume of 50 ul |

The cycling conditions used were

| | |
|---|---|
| | 98° C. 30 seconds |
| 10 cycles of | 98° C. 10 seconds |
| | 58° C. 20 seconds |
| | 68° C. 2 minutes, 30 seconds |
| 20 cycles of | 98° C. 10 seconds |
| | 58° C. 20 seconds |
| | 68° C. 3 minutes |
| | 68° C. 5 minutes |
| | 4° C. hold |

The PCR product (3152 bp) was gel purified using Fermentas GeneJet Gel purification kit (cat # K0691), according to the manufacturer's instructions, with final elution in 40 ul of elution buffer.

The purified V_{H} RT-PCR products were employed as megaprimers with the linearised pUCG3 to give phagemid products for transformation and library creation, based on the following reactions;

| | | |
|---|---|---|
| Phusion GC 2x mix | 25 | μl |
| Linearised pUCG3 | 700 | ng |
| V_{H} PCR product | 250 | ng |
| DMSO | 1.5 | μl |

| | |
|---|---|
| Nuclease-free H₂O | to 50 μl final volume |

PCR was performed as follows;

| | | |
|---|---|---|
| 98° C. 30 sec | | |
| 98° C. 10 sec | | |
| 58° C. 20 sec | {close oversize brace} | 10 cycles |
| 72° C. 2 min | | |
| 72° C. 5 min | | |
| Hold at 10° C. | | |

The products of PCR were analysed on a 1% agarose gel.

The various family V_{H}/phagemid products were purified using Ferment as PCR purification kit (cat #K0702) according to the manufacturer's instructions with the final elution being in 25 ul H₂O and used for transformations of TG1 E. coli (Lucigen, Cat: 60502-2) by electroporation using BioRad® 10×1 mm cuvettes (BioRad® cat #165-2089, a Eppendorf™ Eporator and pre-warmed recovery medium (Lucigen, proprietary mix). 2 ul of the purified products were added to 25 ul of cells for the electroporation, with up to 10 electroporations being performed for each V_{H}/phagemid product at 1800v. Electroporated cells were pooled and recovered in 50 ml Falcon tubes incubated for 1 hour at 37° C. with shaking at 150 rpm. A 10-fold dilution series of an aliquot of the transformations was performed and plated in petri dishes containing 2×TY agar supplemented with 2% (w/v) glucose and 100 ug/ml ampicillin. Resulting colonies on these dishes were used to estimate the library size. The remainder of the transformation was plated on large format Bioassay dishes containing 2×TY agar supplemented with 2% (w/v) glucose and 100 ug/ml ampicillin. All agar plates were incubated overnight at 30° C. 10 ml of 2×TY broth was added to the large format bioassay dishes and colonies were scraped and OD600 measured (OD of 1.0=5×10⁸ cells/ml). Aliquots were stored at −80° C. in cryovials after addition of 50% v/v glycerol solution (50%) or used directly in a phage selection process.

In some instances, clones were picked directly and sequence was determined to give an estimate of the diversity of the library. Typically, for each mouse a phage display library with greater than 1e8 recombinants was constructed to fully capture the V_{H} diversity in that mouse.

### Example 6. Selection Strategies for Isolation of IL-17RA-Binding V_{H}

Preparation of library phage stocks and phage display selections were performed according to published methods (Antibody Engineering, Edited by Benny Lo, chapter 8, p 161-176, 2004). In most cases, phage display combined with a panning approach was used to isolate binding V_{H} domains. However, a variety of different selection methods may be employed, including (a) soluble selections; (b) selections performed under stress, where phage are heated at 70° C. for 2 hours prior to selection; and (c) competitive selections, where excess antigen or antigen-reactive V_{H} domains are added as competition to encourage the recovery of high affinity V_{H} domains or to skew selections away from a particular epitope.

The IL-17RA antigen was expressed as a fusion with the Fc domain of human IgG1. Therefore, to minimise the isolation of unwanted antibodies to the Fc region of the fusion protein, human IgG1 was added to the phage display selections at 100 ug/ml (approx 650 nM) to compete or deselect for Fc binding V_{H}. For panning, antigen was immobilised onto maxisorb plates (Nunc 443404) in 50 ul volumes at 0.1-10 ug/ml in PBS. For the libraries from immunised mice, one round of selection was carried out.

### Example 7. Assays for Target Binding

V_{H} from the different selections were screened in one or more of the following assays to identify specific V_{H} with neutralising properties.

a) Binding ELISA

Following selections of the libraries, specific V_{H} antibodies were identified by phage ELISA following published methods (Antibody Engineering, Edited by Benny Lo, chapter 8, p 161-176, 2004). Phage ELISAs were performed against target protein and an unrelated antigen as control. In some cases, purified or crude extracts of V_{H} domains were assayed by ELISA instead of using a phage ELISA. In these cases, bacterial periplasmic extracts or purified V_{H} were used.

Small-scale bacterial periplasmic extracts were prepared from 1 ml cultures, grown in deep well plates. Starter cultures were used to inoculate 96-well deep well plates (Fisher, cat# MPA-600-030X) containing 2×TY broth (Melford, M2130), supplemented with 0.1% (w/v) glucose+100 ug/ml ampicillin at 37° C. with 250 rpm shaking. When OD600 had achieved 0.6-1, V_{H} production was induced by adding 100 ul of 2×TY, supplemented with IPTG (final concentration 1 mM) and ampicillin and the cultures were grown overnight at 30° C. with shaking at 250 rpm. E. coli were pelleted by centrifugation at 3200 rpm for 10 mins and supernatants discarded. Cell pellets were resuspended in 30-100 ul of ice cold extraction buffer (20% (w/v) sucrose, 1 mM EDTA & 50 mM Tris-HCl pH8.0) by gently pipetting. Cells were incubated on ice for 30 minutes and then centrifuged at 4500 rpm for 15 mins at 4° C. Supernatants were transferred to polypropylene plates and used, following incubation in Marvel/PBS blocking solution, in the ELISA.

The purified V_{H} were obtained by using the V_{H} C-terminal 6×HIS tag for nickel-agarose affinity chromatographic purification of the periplasmic extracts. A starter culture of each V_{H} was grown overnight in 5 ml 2×TY broth (Melford, M2103) supplemented with 2% (w/v) glucose+100 ug/ml ampicillin at 30° C. with 250 rpm shaking. 50 ul of this overnight culture was then used to inoculate 50 ml 2×TY supplemented with 2% (w/v) glucose+100 ug/ml ampicillin and incubated at 37° C. with 250 rpm shaking for approximately 6-8 hours (until OD600=0.6-1.0). Cultures were then centrifuged at 3200 rpm for 10 mins and the cell pellets resuspended in 50 ml fresh 2×TY broth containing 100 ug/ml ampicillin+1 mM IPTG. Shake flasks were then incubated overnight at 30° C. and 250 rpm. Cultures were again centrifuged at 3200 rpm for 10 mins and supernatants discarded. Cell pellets were resuspended in 1 ml ice cold extraction buffer (20% (w/v) sucrose, 1 mM EDTA & 50 mM Tris-HCl pH8.0) by gently pipetting and then a further 1.5 ml of 1:5 diluted ice cold extraction buffer added. Cells were incubated on ice for 30 minutes and then centrifuged at 4500 rpm for 15 mins at 4° C. Supernatants were transferred to 50 ml Falcon tubes containing imidazole (Sigma, 12399—final concentration 10 mM) and 0.5 ml of nickel agarose beads (Qiagen, Ni-NTA 50% soln, 30210) pre-equilibrated with PBS buffer. V_{H} binding to the nickel agarose beads was allowed to proceed for 2 hours at 4° C. with gentle shaking. The nickel agarose beads were then transferred to a polyprep column (BioRad™, 731-1550) and the supernatant discarded by gravity flow. The columns were then washed 3 times with 5 ml of PBS+0.05% Tween™ followed by 3 washes with 5 ml of PBS containing imidazole at a concentration of 20 mM. V_{H} were then eluted from the columns by the addition of 250 ul of PBS containing imidazole at a concentration of 250 mM. Imidazole was then removed from the purified V_{H} preparations by buffer exchange with NAP-5 columns (GE Healthcare, 17-0853-01) and then eluting with 1 ml of PBS. Yields of purified V_{H} were estimated spectrophotemetrically and purity was assessed using SDS PAGE.

The binding ELISA for crude or purified V_{H} was similar to the serum ELISA and phage ELISA, previously described, mostly differing in the final detection steps. Briefly, antigen was immobilised on maxisorb plates (Nunc 443404) by adding 50 ul volumes at 0.1-1 ug/ml in PBS and incubating at 4° C. overnight. Following coating, the antigen solution was aspirated and the plates were washed using PBS (prepared from PBS tablets, Oxoid cat no. BR0014G) supplemented with 0.05% Tween® 20 (sigma P1379), followed by washes with PBS without added Tween®. To block non-specific protein interactions, a solution of 3% skimmed milk powder (Marvel) in PBS was added to the wells and the plate was incubated for at least one hour at room temperature. Dilutions of periplasmic extract or purified V_{H} in 3% Marvel/PBS were prepared in polypropylene tubes or plates and incubated for at least one hour at room temperature prior to transfer to the blocked ELISA plate where a further incubation of at least one hour took place. Unbound protein was then washed away using repetitive washes with PBS/Tween® followed by PBS. A solution of HRP-conjugated anti-His Ab (Miltenyi Biotec, 130-092-785), prepared at 1:1000 dilution in PBS/3% Marvel was then added to each well and a further incubation at room temperature for at least one hour took place. Unbound detection antibody was removed by repeated washing using PBS/Tween® and PBS. The ELISA was then developed using TMB substrate (Sigma cat. no. T0440) and the reaction was stopped after 10 minutes by the addition of 0.5M H₂SO₄ solution (Sigma cat. no. 320501). Absorbances were determined by reading at 450 nm. Example ELISA data is shown in FIG. 14.

b) R/L Biochemical Inhibition Assay

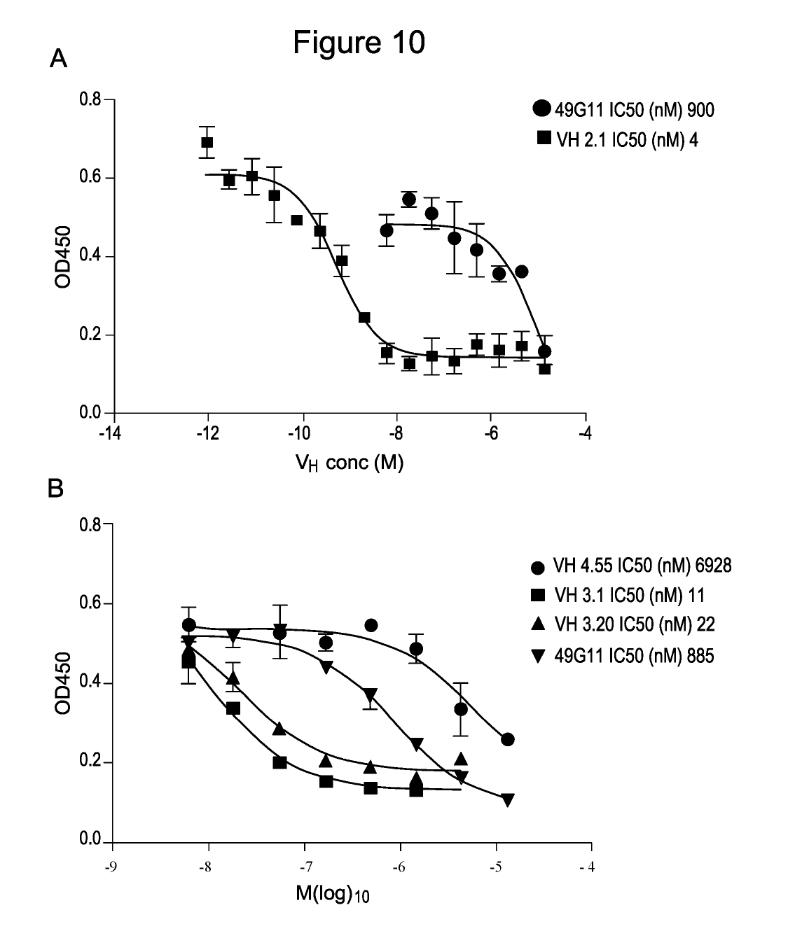

V_{H}, both purified and crude periplasmic extracts, were also tested for their ability to inhibit the interaction of IL-17A with recombinant IL-17RA-Fc. Maxisorb 96F well mictrotitre plates were incubated with 50 ul solution of 2 nM IL-17-RA (R & D systems, cat #177-IR-100) and incubated overnight at 4° C. Following washing of excess coating antigen, as described above, the wells of the plate were incubated with 3% Marvel/PBS to block non-specific protein interactions. V_{H} preparations, crude periplasmic extracts or purified V_{H}, or suitable controls, were prepared with 1 nM recombinant IL-17A (Peprotech, cat# AF-200-17) in 3% skimmed milk powder/PBS solution in polypropylene plates or tubes. The mixture was then transferred to the assay plate and incubated for 1 hour at room temperature. Excess protein was removed by washing and bound IL-17A was detected by incubation with biotinylated anti-IL-17A Mab (R & D Systems, cat BAF317) followed by the addition of neutravidin-HRP (Pierce, cat#31030) and TMB substrate (Sigma, cat# T0440). The TMB reaction was stopped by addition of 0.5M H₂SO₄ and absorbances were measured at 450 nm in a plate reader.

Where appropriate, curve fitting in PRISM was used to determine the EC₅₀ of inhibiting V_{H}. Example data illustrating inhibition of IL-17A responses in the biochemical assay are shown in FIG. 10. V_{H} were expressed from phagemid vector and have the following C terminal extension LEGGGS HHHHHH (SEQ ID NO.2606).

c) R/L Cell Based Inhibition Assay

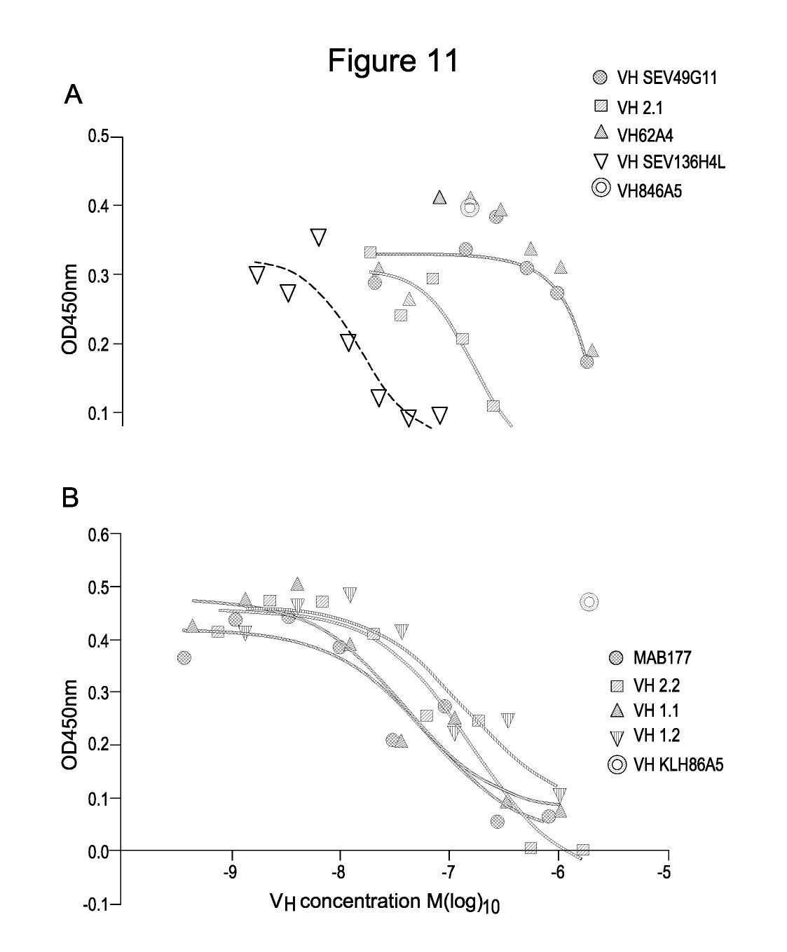

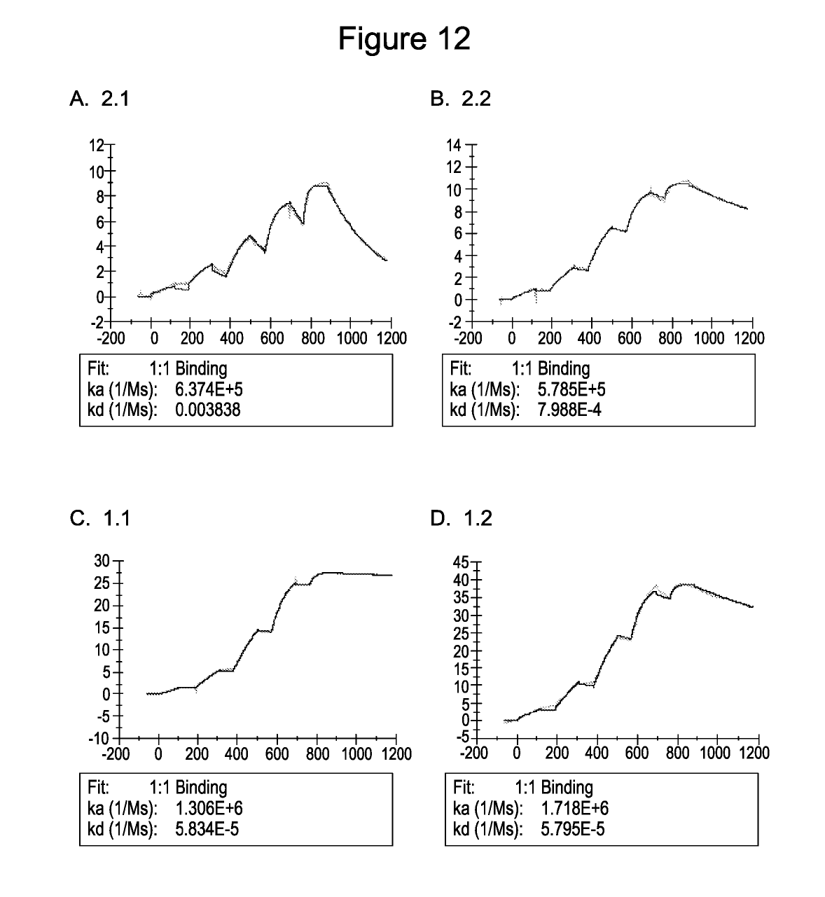

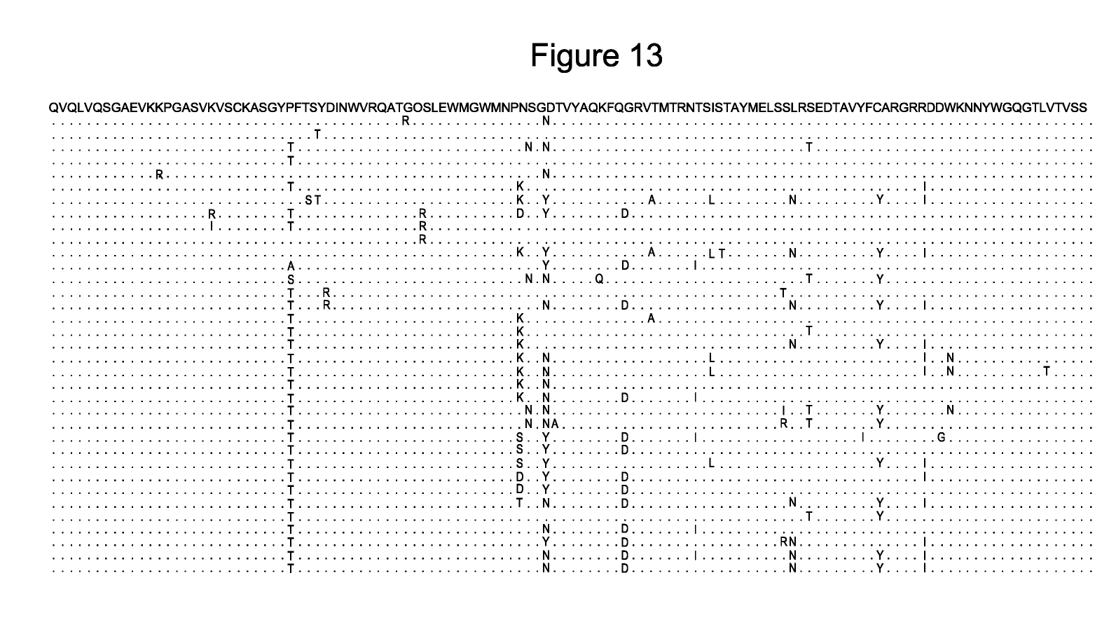

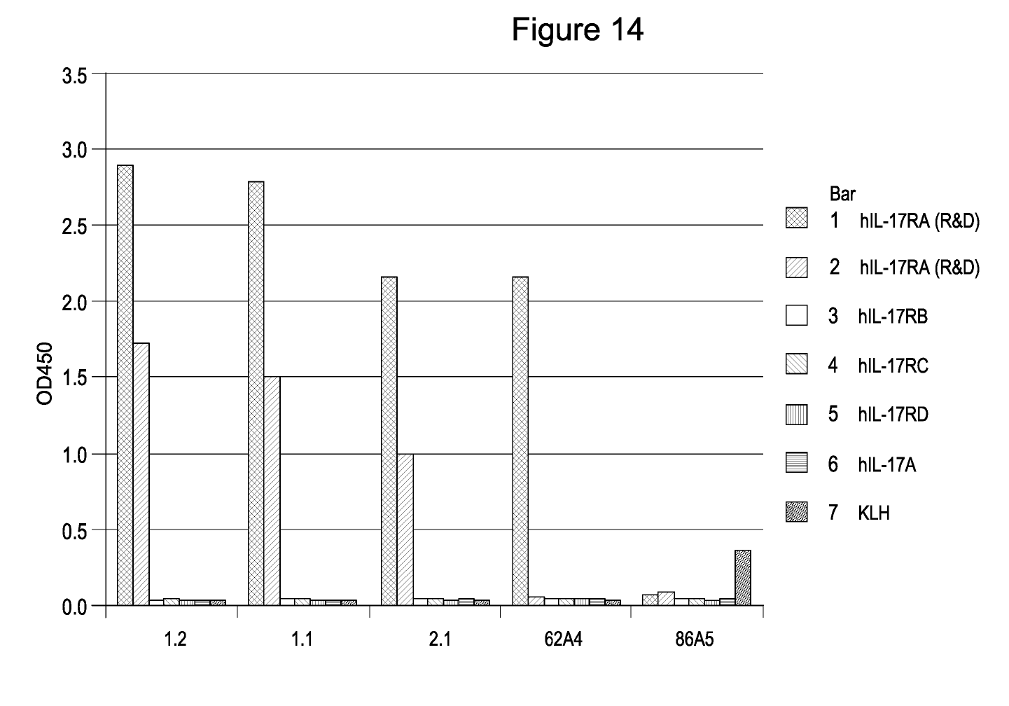

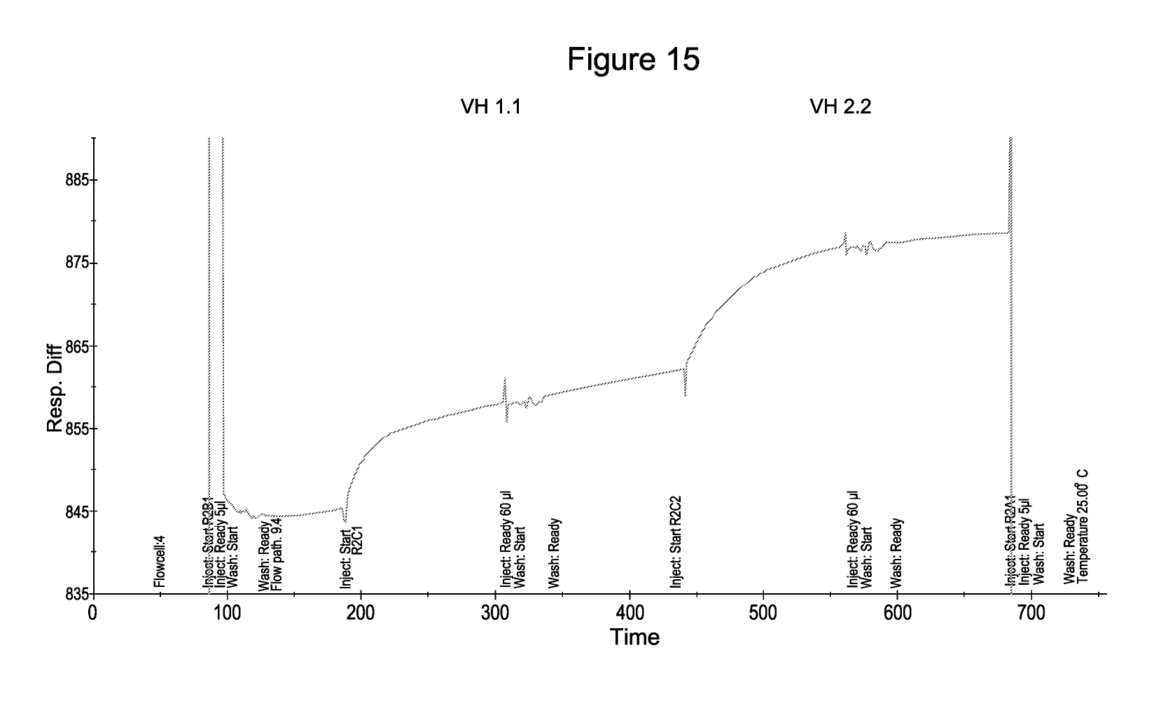

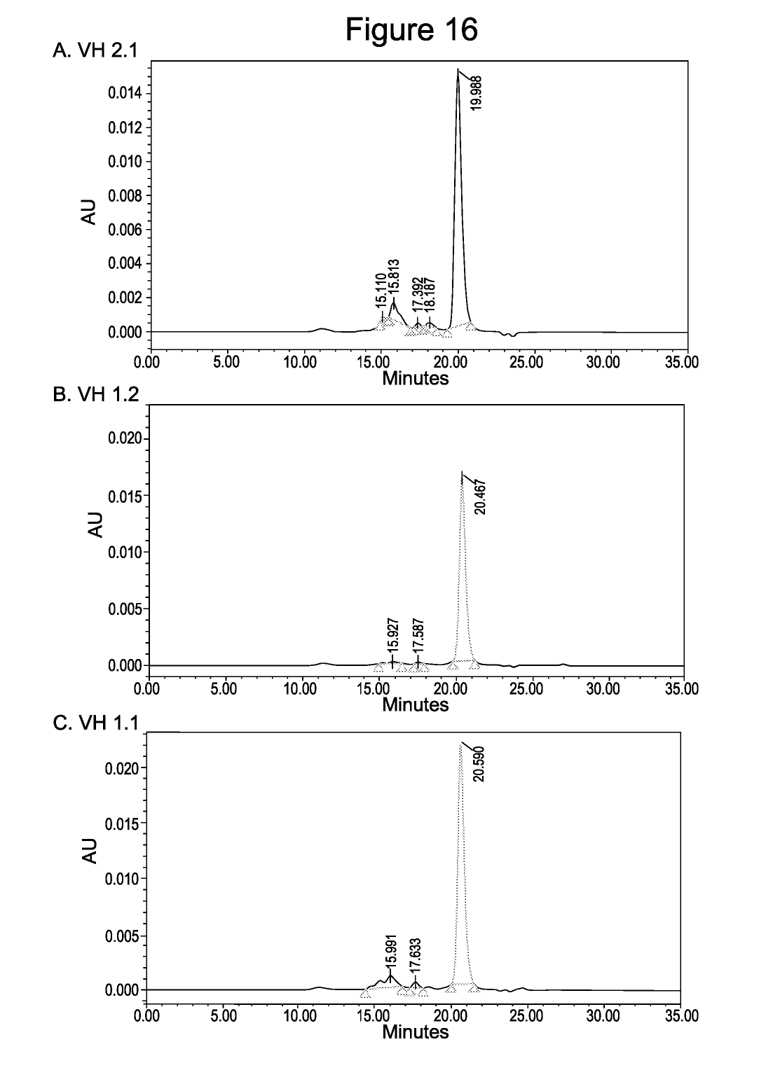

An assay was developed to measure the ability of IL-17RA-binding V_{H} to inhibit IL-17A-induced IL6 release from the cell line, HT1080 (ECACC cat #85111505). The cell line was maintained in exponential growth in MEM with Earles's salts, supplemented with non-essential amino acids, 10% FBS, 2 mM L-Glutamine and penicillin/streptomycin and incubated in a humidified incubator at 37° C., 5% CO₂. For the assay, 50,000 cells/well were seeded into a 96 flat bottomed tissue culture plate and cultured overnight. Serial dilution of purified V_{H} were prepared and incubated at 37° C. for 1 hour with culture medium/PBS supplemented with 10 ng/ml IL-17A (Peprotech cat# AF200-17). Following incubation, the V_{H}/IL-17A mixture (or suitable controls) were transferred to the HT1080 cells (from which culture medium had been aspirated) and incubated for a further 5 hours in the CO₂ incubator. The cell culture supernatant was collected and assayed for IL6 using the IL-6 Duoset (R & D Systems, cat# DY206), following manufacturer's instructions. Example data illustrating inhibition of IL-17A responses in the cell based assay are shown in FIG. 11. V_{H} were expressed from phagemid vector and have the following C terminal extension LEGGGS HHHHHH.

d) Biacore®.

Binding kinetics of anti-IL-17RA V_{H} antibodies were measured on a BIAcore® T200 instrument. For IL-17RA, first a protein G chip was prepared by diluting protein G to 20 ug/ml in acetate buffer, pH 4 (BIAcore®, cat# BR-100-49) and then coupled 1200 RU to a CM5 Series S chip using amine coupling chemistry. This surface was then used to capture IL-17RA Fc fusion protein from solution: IL-17RA at 10 ug/ml in HBS injected for 10 seconds at 30 ul/min flow rate would capture approximately 100-150 RU of IL-17RA onto the protein G surface.

Binding kinetics of anti-IL-17RA V_{H} antibodies were determined by single-cycle kinetics. V_{H} antibodies were prepared in dilution series (typically 1:3 dilution series starting with 100 nM V_{H} at the highest concentration), and then injected over the antigen coated surfaces and also a blank surface, starting with the lowest concentration of V_{H} and then working progressively up to the highest concentration. V_{H} binding kinetics were then determined from the (blank subtracted) sensorgram traces using 1:1 binding models and BIAevaluation software. Example BIAcore® binding traces are shown in FIG. 12.

Following the above screening cascade, a number of V_{H} to IL-17R were identified that demonstrated inhibitory properties. These are summarised below in table 9. The clones are the parent clones for optimisation.

TABLE 9

| | | | | | Cell | |
|---|---|---|---|---|---|---|
| | | | | Biochem | assay | BIAcore |
| | | Clone | | IC50 | IC50 | (Affinity) |
| | | name | CDR3 sequence | (nM) | (nM) | KD (M) |
| IL- | Immunised | 2.1 | GRRDDWKNNY | 3.7 | 280 | 7.0E-09 |
| 17RA | mouse | | SEQ ID NO 1267 | | | |
| | | 1.1 | EKGLGFCRGGSCSY | nd | 4.7 | 6.3E-11 |
| | | | FDY | | | |
| | | | SEQ ID NO 3 | | | |
| | | 5.1 | NGARYNWNYGDFQH | 80 | 73 | 4.1E-08 |
| | | | SEQ ID NO 2559 | | | |
| | | 4.1 | GGYNAWRTDY | nd | 282 | 4.5E-09 |
| | | | SEQ ID NO 2131 | | | |
| | | 3.1 | GWESGWFEP | 11 | Weak | 9.6E-09 |
| | | | SEQ ID NO 1767 | | | |
| | | 6.1 | KDITNIAVGSLGY | nd | 233 | 3.1E-08 |
| | | | SEQ ID NO 2575 | | | |
| | | 7.1 | SRDWGSRAFDI | nd | 1840 | 3.7E-07 |
| | | | SEQ ID NO 2579 | | | |

### Example 8. Optimisation of V_{H}

a. Optimisation of V_{H} Isolated from Immunised Mice

Where appropriate, a novel optimisation strategy was used to increase binding affinities of V_{H} isolated from immunised mice. Lead VH were aligned with other members of the same lineage to identify somatic hypermutation hot-spots targeted during the immune response (FIG. 13). The choice of amino acids at these positions formed the basis of a new recombination library approach, and led to the design of new libraries aimed at selecting higher affinity V_{H} with optimal amino acids at each mutation hot-spot.

As an example for IL-17RA, clone 2.1 was isolated directly from immunised TKO mouse. This V_{H} was shown to bind IL-17RA with high affinity Alignment of clone 2.1 with other members of the same lineage identified a number of amino acid positions that had been mutated during the immune response, and both V_{H}-CDRs and V_{H}-framework regions were affected. This information was then utilised to design a new clone 2.1 recombination library with the aim of identifying a higher affinity variant of clone 2.1. Following construction and phage display selection of a recombination library a new variant was isolated (2.2) that was improved in affinity by 10-fold.

Phusion High fidelity PCR master mix with HF buffer (cat # F531L, NEB) was used for the PCR reactions which were set up for each primer pairing as follows:

| | |
|---|---|
| Phusion HF 2x mix | 25 μl |
| Primers (10 uM) | 1.25 μl each (pairings as in table) |
| 53F9 plasmid DNA (34 ng/ul) | 0.5 μl |
| Nuclease-free H₂O | to 50 ul final volume |

PCR was performed as follows;

| | | |
|---|---|---|
| 98° C. 30 sec | | |
| 98° C. 10 sec | | |
| 58° C. 20 sec | {close oversize brace} | 31 cycles |
| 72° C. 20 sec | | |
| 72° C. 10 min | | |
| Hold at 10° C. | | |

The products of each PCR were analysed on a 1% agarose gel. Each product was then purified using Fermentas PCR purification kit (K0701) into 40 ul elution buffer. Assembly PCRs were then set up to rebuild the full V_{H} sequence:

| | |
|---|---|
| Phusion HF 2x mix | 25 μl |
| Purified PCR product 1 | 5 μl |
| Purified PCR product 2 | 5 μl |
| Purified PCR product 3 | 5 μl |
| Purified PCR product 4 | 5 μl |
| Purified PCR product 5 | 5 μl |

PCR was performed as follows;

| | | |
|---|---|---|
| 98° C. 30 sec | | |
| 98° C. 10 sec | | |
| 58° C. 20 sec | {close oversize brace} | 5 cycles |
| 72° C. 20 sec | | |

Added 0.5 ul of primers V3/pelB (long) and VH_J/F (long) (both 10 uM) to the reaction and then continued for a further 10 PCR cycles at the above conditions. The PCR product was analysed on a 1% agarose gel and purified using Fermentas PCR purification kit into 40 ul elution buffer. The PCR product was then used as a megaprimer for library construction as described above in Example 5, part b. Phage display selections and V_{H} screening was then performed as described in examples 7 and 8, following which several new variants of clone 2.1 were isolated with up to 10-fold improved affinities.

Following the lead optimisation steps, the potencies of improved V_{H} were as follows:

TABLE 10
a) V_{H} produced following optimisation
of anti-IL-17RA V_{H} family 2

| | | | Cell | | | |
|---|---|---|---|---|---|---|
| | | Biochem | assay | | | |
| VH | | IC50 | IC50 | BIAcore (Affinity) | | |
| name | CDR3 sequence | (nM) | (nM) | ka (1/Ms) | kd (1/s) | KD (M) |
| 2.1 | GRRDDWKNNY | 3.7 | 280 | 3.68E+05 | 2.63E-03 | 7.0E-09 |
| | SEQ ID NO 1267 | | | | | |
| 2.3 | GRRDNWKNNY | 1.3 | 118 | 5.55E+05 | 0.001368 | 2.50E-09 |
| | SEQ ID NO 1275 | | | | | |
| 2.2 | GRRDDWKNNY | 1.4 | 61 | 5.79E+05 | 7.99E-04 | 1.40E-09 |
| | SEQ ID NO 1271 | | | | | |

Optimised V_{H} show improved affinities to IL-17R and improved potencies in the IL-17R cell based assay due to slower off-rates (FIG. 15).

### Example 9—Characterisation of V_{H}

a. Specificity of Anti-IL-17A

The specificity of individual V_{H} for target antigen was confirmed by ELISA, following the methods described in Example 8(a). V_{H} were tested for binding to IL-17RA and shown not to cross-react with close relatives such as IL-17RB, IL-17RC and IL-17RD. (FIG. 14).

b. Epitope Mapping

VH were shown to bind to unique epitopes of IL-17RA using a BIAcore T200 instrument. Manual sensorgrams were initiated at 30 ul/min in HBS buffer and V_{H} injected as appropriate over the IL-17RA coupled CM5 chip coupled CM5 chip, plus a blank surface for reference subtraction (FIG. 15).

For IL-17RA, IL-17RA-Fc fusion protein was first captured on a protein G chip by injecting IL-17RA-Fc at 10 ug/ml in HBS buffer for 10 seconds. This surface was then used to measure binding and competition between different anti-IL-17RA V_{H} following the method described above. Epitope competition data is presented for 1.1 and 2.2 (FIG. 15). V_{H} were expressed from phagemid vector and have the following C terminal extension LEGGGS HHHHHH (SEQ ID NO.2606).

c. HPLC Size Exclusion Chromatography

Purified V_{H} (clones 1.1, 1.2, 2.1) were subjected to size exclusion chromatography. Briefly, purified V_{H} were analysed using a Water® 2795 Separation Module with a Waters® 2487 Dual λ #absorbance Detector—(Detected at 280 nM) and a TSKgel G2000SWXL (TOSOH) column. Samples were injected in 10-50 ul volumes and run in mobile phases of either 10% isopropanol/90% PBS or 100 mM Phosphate buffer, pH 6.8, 150 mM NaCl at a flow rate of 0.5 ml/min-0.7 ml/min. Data were collected for up to 35 minutes and the size of the V_{H} fraction compared with known standards (see FIG. 16). V_{H} were expressed from phagemid vector and have the following C terminal extension LEGGGS HHHHHH (SEQ ID NO.2606).

1. A binding molecule that binds human IL-17RA, comprising a human heavy chain variable immunoglobulin domain (V_{H}) that comprises a CDR1, a CDR2, and a CDR3 sequence,
a) wherein said CDR3 sequence comprises the amino acid sequence of SEQ ID NO. 3, or an amino acid sequence with at least 70%, at least 80%, at least 90%, or at least 95% homology thereto; or
b) wherein said CDR3 sequence comprises the amino acid sequence of SEQ ID NO. 1267, or an amino acid sequence with at least 70%, at least 80%, at least 90%, or at least 95% homology thereto; or
c) wherein said CDR3 sequence comprises the amino acid sequence of SEQ ID NO. 1767, or an amino acid sequence with at least 70%, at least 80%, at least 90%, or at least 95% homology thereto; or
d) wherein said CDR3 sequence comprises the amino acid sequence of SEQ ID NO. 2131, or an amino acid sequence with at least 70%, at least 80%, at least 90%, or at least 95% homology thereto; or
e) wherein said CDR3 sequence comprises the amino acid sequence of SEQ ID NO. 2559, or an amino acid sequence with at least 70%, at least 80%, at least 90%, or at least 95% homology thereto; or
f) wherein said CDR3 sequence comprises the amino acid sequence of SEQ ID NO. 2575, or an amino acid sequence with at least 70%, at least 80%, at least 90%, or at least 95% homology thereto; or
g) wherein said CDR3 sequence comprises the amino acid sequence of SEQ ID NO. 2579, or an amino acid sequence with at least 70%, at least 80%, at least 90%, or at least 95% homology thereto; and
wherein said molecule has a KD (M) value in the range of from 6×10⁻¹¹ to 3×10⁻⁷ when assessed by BIAcore.

2. The binding molecule according to claim 1 wherein said molecule is an immunoglobulin single domain antibody.

3. The binding molecule according to claim 1,
a) wherein said molecule comprises the amino acid sequence of SEQ ID NO. 3, or said amino acid sequence with homology thereto, and said CDR1 sequence comprises the amino acid sequence of SEQ ID NO. 1, or an amino acid sequence with at least 70%, at least 80%, at least 90%, or at least 95% homology thereto and said CDR2 sequence comprises the amino acid sequence of SEQ ID NO. 2, or an amino acid sequence with at least 70%, at least 80%, at least 90%, or at least 95% homology thereto; or
b) wherein said molecule comprises the amino acid sequence of SEQ ID NO. 1267, or said amino acid sequence with homology thereto, and said CDR1 sequence comprises the amino acid sequence of SEQ ID NO. 1265, or an amino acid sequence with at least 70%, at least 80%, at least 90%, or at least 95% homology thereto, and said CDR2 sequence comprises the amino acid sequence of SEQ ID NO. 1266, or an amino acid sequence with at least 70%, at least 80%, at least 90%, or at least 95% homology thereto; or
c) wherein said molecule comprises the amino acid sequence of SEQ ID NO. 1767, or said amino acid sequence with homology thereto, and said CDR1 sequence comprises the amino acid sequence of SEQ ID NO. 1765, or an amino acid sequence with at least 70%, at least 80%, at least 90%, or at least 95% homology thereto, and said CDR2 sequence comprises the amino acid sequence of SEQ ID NO. 1766, or an amino acid sequence with at least 70%, at least 80%, at least 90%, or at least 95% homology thereto; or
d) wherein said molecule comprises the amino acid sequence of SEQ ID NO. 2131, or said amino acid sequence with homology thereto, and said CDR1 sequence comprises the amino acid sequence of SEQ ID NO. 2129, or an amino acid sequence with at least 70%, at least 80%, at least 90%, or at least 95% homology thereto, and said CDR2 sequence comprises the amino acid sequence of SEQ ID NO. 2130, or an amino acid sequence with at least 70%, at least 80%, at least 90%, or at least 95% homology thereto; or
e) wherein said molecule comprises the amino acid sequence of SEQ ID NO. 2559, or said amino acid sequence with homology thereto, and said CDR1 sequence comprises the amino acid sequence of SEQ ID NO. 2557, or an amino acid sequence with at least 70%, at least 80%, at least 90%, or at least 95% homology thereto, and said CDR2 sequence comprises the amino acid sequence of SEQ ID NO. 2558, or an amino acid sequence with at least 70%, at least 80%, at least 90%, or at least 95% homology thereto; or
f) wherein said molecule comprises the amino acid sequence of SEQ ID NO. 2575, or said amino acid sequence with homology thereto, and said CDR1 sequence comprises the amino acid sequence of SEQ ID NO. 2573, or an amino acid sequence with at least 70%, at least 80%, at least 90%, or at least 95% homology thereto, and said CDR2 sequence comprises the amino acid sequence of SEQ ID NO. 2574, or an amino acid sequence with at least 70%, at least 80%, at least 90%, or at least 95% homology thereto; or
g) wherein said molecule comprises the amino acid sequence of SEQ ID NO. 2579, or said amino acid sequence with homology thereto, and said CDR1 sequence comprises the amino acid sequence of SEQ ID NO. 2577, or an amino acid sequence with at least 70%, at least 80%, at least 90%, or at least 95% homology thereto, and said CDR2 sequence comprises the amino acid sequence of SEQ ID NO. 2578, or an amino acid sequence with at least 70%, at least 80%, at least 90%, or at least 95% homology thereto.

4. The binding molecule according to claim 3,
(a) wherein said CDR1 sequence comprises or consists of the amino acid sequence of SEQ ID NO. 1, 5, 9, 13, 17 or 21, said CDR2 sequence comprises or consists of the amino acid sequence of SEQ ID NO. 2, 6, 10, 14, 18 or 22, and said CDR3 sequence comprises or consists of the amino acid sequence of SEQ ID NO: 3, 7, 11, 15, 19 or 23; or
(b) wherein said CDR1 sequence comprises or consists of the amino acid sequence of SEQ ID NO. 1265, 1269, or 1273, said CDR2 sequence comprises or consists of the amino acid sequence of SEQ ID NO. 1266, 1270, or 1274, and said CDR3 comprises or consists of the amino acid sequence of SEQ ID NO. 1267, 1271, or 1275;
c) wherein said CDR1 sequence comprises or consists of the amino acid sequence of SEQ ID NO. 1765, said CDR2 sequence comprises or consists of the amino acid sequence of SEQ ID NO. 1766, and said CDR3 sequence comprises or consists of the amino acid sequence SEQ ID NO: 1767;
d) wherein said CDR1 sequence comprises or consists of the amino acid sequence of SEQ ID NO. 2129, said CDR2 sequence comprises or consists of the amino acid sequence of SEQ ID NO. 2130, and said CDR3 sequence comprises or consists of the amino acid sequence SEQ ID NO: 2131;
e) wherein said CDR1 sequence comprises or consists of the amino acid sequence of SEQ ID NO. 2557, said CDR2 sequence comprises or consists of the amino acid sequence of SEQ ID NO. 2558, and said CDR3 sequence comprises or consists of the amino acid sequence SEQ ID NO: 2559;
f) wherein said CDR1 sequence comprises or consists of the amino acid sequence of SEQ ID NO. 2573, said CDR2 sequence comprises or consists of the amino acid sequence of SEQ ID NO. 2574, and said CDR3 sequence comprises or consists of the amino acid sequence SEQ ID NO: 2575; or
(g) wherein said CDR1 sequence comprises or consists of the amino acid sequence of SEQ ID NO. 2577, said CDR2 sequence comprises or consists of the amino acid sequence of SEQ ID NO. 2578, and said CDR3 sequence comprises or consists of the amino acid sequence SEQ ID NO: 2579.

5. (canceled)

6. The binding molecule according to claim 1 wherein said V_{H} domain comprises or consists of the amino acid sequence of SEQ ID NO. 4, 1268, 1768, 2132, 2560, 2576, or 2580, or an amino acid sequence with at least 70%, 80%, 90% or 95% homology to any one of said amino acid sequences; or wherein said V_{H} domain comprises or consist of the amino acid sequence of SEQ ID NO. 8, 12, 16, 20, or 24.

7-46. (canceled)

47. The binding molecule according to claim 1, wherein said binding molecule further comprises:
one or more additional V_{H} domains; and/or
a conjugated toxin, enzyme, or radioisotope.

48-50. (canceled)

51. A binding molecule that competes for binding to human IL-17RA with the binding molecule of claim 1.

52. A pharmaceutical composition comprising the binding molecule according to claim 1 and a pharmaceutical carrier.

53. The pharmaceutical composition according to claim 52,
wherein the composition is formulated for administration topically to the skin; and/or
wherein the composition further comprises a chemical skin penetration enhancer.

54. A method for treating at least one disease or condition selected from the group consisting of an autoimmune disease, an inflammatory condition, an allergy, an allergic condition, a hypersensitivity reaction, a severe infection, and an organ or tissue transplant rejection, comprising:
administering an effective amount of the pharmaceutical composition according to claim 52.

55-63. (canceled)

64. The method according to claim 54 wherein said disease or condition is at least one selected from the group consisting of: psoriasis, spondyloarthropathies, uveitis, atopic dermatitis, systemic lupus erythematosis, rheumatoid arthritis, osteoarthritis, juvenile chronic arthritis, systemic sclerosis, idiopathic inflammatory myopathies, Sjogren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central and peripheral nervous systems, multiple sclerosis, idiopathic demyelinating polyneuropathy, Guillain Barre syndrome, chronic inflammatory demyelinating polyneuropathy, a hepatobiliary disease, infectious hepatitis, autoimmune chronic active hepatitis, primary biliary cirrhosis, granulomatous hepatitis, sclerosing cholangitis, inflammatory bowel disease, gluten-sensitive enteropathy, Whipple's disease, an autoimmune skin disease, an immune-mediated skin disease, bullous skin disease, erythema multiforme, contact dermatitis, disease, asthma, allergic rhinitis, food hypersensitivity, urticaria, an immunologic disease of the lung, eosinophilic pneumonia, idiopathic pulmonary fibrosis, hypersensitivity pneumonitis, an autoimmune haematological disorder, hemolytic anaemia, aplastic anaemia, pure red cell anaemia, idiopathic thrombocytopenia, autoimmune inflammatory bowel disease, ulcerative colitis, Crohn's disease, Irritable Bowel Syndrome, a transplantation associated disease, graft rejection, and graft-versus-host-disease.

65. An in vivo or in vitro method for reducing human IL-17RA activity comprising contacting human IL-17RA with the binding molecule according to claim 1.

66. A method for determining the presence of IL-17RA in a test sample by an immunoassay comprising:
contacting said sample with the binding molecule according to claim 1 and at least one detectable label.

67. (canceled)

68. An isolated nucleic acid molecule comprising a nucleotide sequence encoding the binding molecule according to claim 1.

69. A nucleic construct comprising the nucleic acid according to claim 68.

70. An isolated host cell comprising the construct according to claim 69.

71. A method for producing the binding molecule according to claim 1 comprising expressing a nucleic acid encoding the binding molecule in a host cell and isolating the binding molecule from the host cell culture.

72. A kit comprising the binding molecule according to claim 1.

73. A method for producing a binding molecule comprising at least one immunoglobulin single domain antibody that binds IL-17RA, wherein said domain is a human V_{H} domain, said method comprising
a) immunizing a transgenic mouse with an IL-17RA antigen, wherein said transgenic mouse expresses a nucleic acid construct comprising human heavy chain V_{H} genes and is not capable of making functional endogenous light or heavy chains;
b) generating a library from said mouse, wherein said library comprises amino acid sequences that comprise V_{H} domain amino acid sequences; and
c) isolating at least one amino acid sequence comprising said V_{H} domain amino acid sequence from said library,
thereby obtaining said binding molecule comprising a single domain antibody that comprises a human V_{H} domain for binding IL-17RA.

74. A biparatopic, bivalent, or multispecific binding molecule comprising the binding molecule according to claim 1.
